(19) United States
(12) Patent Application Publication
(10) Pub. No.: US 2026/0284370 A1
(43) Pub. Date: Sep. 24, 2026

(54) Portable Hydrogel Electrochemical Device for Skin Disease Treatment

(71) Applicant: The University of Chicago, Chicago, IL (US)

(72) Inventors: Bozhi Tian, Chicago, IL (US); Wen Li, Chicago, IL (US)

(21) Appl. No.: 19/571,251

(22) Filed: Mar. 18, 2026

Related U.S. Application Data
(60) Provisional application No. 63/774,369, filed on Mar. 19, 2025.

(51) Int. Cl. A61M 35/00

(52) U.S. Cl. CPC A61M 35/30; A61M 2202/02

(57) ABSTRACT

Systems and methods for generating and delivering molecular hydrogen are disclosed which utilize an electrochemical cell, an adhesive, and a wireless electronic circuit. The electrochemical cell includes a hydrogel, a membrane electrode assembly (MEA) embedded in the hydrogel, and a proton exchange membrane (PEM). The MEA has a first mesh electrode and a second mesh electrode. The PEM is disposed between the first mesh electrode and the second mesh electrode. The adhesive is coupled with a first side of the electrochemical cell, wherein the adhesive secures the electrochemical cell to a tissue of a subject. The wireless electronic circuit is coupled to the electrochemical cell on a second side opposite the first side of the electrochemical cell.

### CROSS REFERENCE TO RELATED APPLICATIONS

This application claims the benefit of priority from U.S. provisional application No. 63/774,369, filed Mar. 19, 2025, which is incorporated by reference in its entirety.

### STATEMENT REGARDING FEDERALLY SPONSORED RESEARCH

This invention was made with government support under EB036091 awarded by the National Institutes of Health, and W911NF-24-1-0053 awarded by the Army Research Laboratory—Army Research Office. The government has certain rights in the invention.

### FIELD

An apparatus and method of generating, storing, and delivering molecular gas is disclosed. The apparatus can be advantageously used for production of hydrogen to provide localized disease treatment.

### BACKGROUND

Hydrogen (H₂) gas is integral to numerous applications, including fuel cells1, industrial chemical synthesis, and biomedical therapies. The small molecular size nature and flammability of H₂ poses significant challenges in storage and release for both industrial and biomedical applications. Traditional production methods, such as steam reforming or electrolysis4, require harsh physical or chemical conditions, bulky equipment, and storage under low temperatures or high pressures4. Therapeutic applications, which leverage the ability of H₂ to neutralize reactive oxygen species (ROS) for protection against ischemia-reperfusion (I/R) injury3,5, are incompatible with such processes and demand mild, portable and controllable delivery systems.

Current H₂ delivery methods in clinical trials, such as gas inhalation and hydrogen-enriched water, suffer from rapid leakage, difficulty in concentration control, and poor tissue specificity. Alternative approaches like photocatalysis8, algae-bacteria systems, and galvanic cells are promising in mice, but exhibit limited external control and inconsistent production rates under dynamic biological conditions, rendering them unsuitable for precise and sustained therapy. These challenges underscore the need for advanced electrochemical engineering approaches that leverage material engineering, interfacial kinetics, and molecular transport to enable controlled H₂ production and sustainable delivery for biomedical applications.

### SUMMARY

In one aspect an electrochemical cell is provided. The electrochemical cell includes a hydrogel, a membrane electrode assembly (MEA), and a proton exchange membrane (PEM). The MEA is embedded in the hydrogel, the MEA having a first mesh electrode and a second mesh electrode. The PEM is disposed between the first mesh electrode and the second mesh electrode.

In some embodiments, the hydrogel is a polyvinyl alcohol (PVA) hydrogel. In some embodiments the PVA hydrogel comprises a polymer concentration between 3% and 10%.

In some embodiments, the first mesh electrode comprises a titanium-platinum alloy (Ti—Pt) and the second mesh electrode comprises a Ti—IrO₂ electrode.

In some embodiments, the PEM comprises a sulfonated tetrafluoroethylene based fluoropolymer-copolymer.

In another aspect, a system for generating and delivering molecular hydrogen includes an electrochemical call, an adhesive, and a wireless electronic circuit. The electrochemical cell includes a hydrogel, a membrane electrode assembly (MEA) embedded in the hydrogel, and a proton exchange membrane (PEM). The MEA includes a first mesh electrode and a second mesh electrode. The PEM is disposed between the first mesh electrode and the second mesh electrode. The adhesive is coupled with a first side of the electrochemical cell, wherein the adhesive secures the electrochemical cell to a tissue of a subject. The wireless electronic circuit is coupled to the electrochemical cell on a second side opposite the first side of the electrochemical cell.

In some embodiments, the adhesive comprises an adherent file. In some embodiments, the adherent film comprises an acrylic-adhesive polyurethane film.

In some embodiments, the first mesh electrode comprises a titanium-platinum alloy (Ti—Pt) and the second mesh electrode comprises an iridium-titanium oxide (Ti—IrO₂) electrode.

In some embodiments, the wireless electronic circuit is a flexible printed circuit board (PCB).

In some embodiments, the PCB includes a microchip that is operably coupled to the PCB having a microcontroller, and the microcontroller comprises one or more processors programmed to perform microcontroller operations including: controlling a digital-to-analog converter (DAC) to regulate current through a Howland circuit contained on the PCB to the MEA of the electrochemical cell. In some embodiments, controlling the DAC to regulate current through the Howland circuit contained on the PCB comprises ensuring controlled H₂ production. In some embodiments, the one or more processors are programmed to perform microcontroller operations further including recording a voltage measurement from the MEA of the electrochemical cell. In some embodiments the one or more processors are programmed to perform microcontroller operations further including operating a communication system of the PCB. In some embodiments, the PCB further includes a battery and a voltage regulator.

In some embodiments, the system further includes a polyimide film disposed between the electrochemical cell and the wireless electronic circuit.

In some embodiments, the system further includes a housing, wherein the housing encompasses the wireless electronic circuit.

In another aspect, a method of generating, storing, and delivering molecular hydrogen (H₂) is provided. The method includes: facilitating a hydrogen evolution reaction (HER) within an electrochemical cell to generate H₂; storing, within the hydrogel of the electrochemical cell, the generated H₂; regulating, by the electrochemical cell, delivery of the stored H₂ from the hydrogel to a tissue of the subject using a wireless electronic circuit attached to the electrochemical cell. The electrochemical cell includes a hydrogel, a membrane electrode assembly (MEA) embedded in the hydrogel, and a proton exchange membrane (PEM). The MEA has a first mesh electrode and a second mesh electrode. The proton exchange membrane (PEM) is disposed between the first mesh electrode and the second mesh electrode.

In some embodiments, the wireless electronic circuit includes a microchip that is operably coupled to the wireless electronic circuit having a microcontroller, and the microcontroller comprises one or more processors programmed to perform microcontroller operations including controlling a digital-to-analog converter (DAC) to regulate current through a Howland circuit contained on the wireless electronic circuit to the MEA of the electrochemical cell.

In some embodiments, controlling the DAC to regulate current through the Howland circuit contained on the wireless electronic circuit comprises ensuring the facilitation of the HER for controlling H₂ generation.

These, as well as other aspects, advantages, and alternatives will become apparent to those of ordinary skill in the art by reading the following detailed description with reference where appropriate to the accompanying drawings. Further, it should be understood that the description provided in this summary section and elsewhere in this document is intended to illustrate the claimed subject matter by way of example and not by way of limitation.

### BRIEF DESCRIPTION OF THE DRAWINGS

FIG. 1 illustrates a schematic diagram of a gas delivery system including a membrane electrode assembly-hydrogel.

FIG. 2 (a)-(b) illustrates a representative process for hydrogen generation, storage and diffusion in a hydrogel electrochemical cell, according to some embodiments. In (a), hydrogen generation (via the hydrogen evolution reaction (HER)), storage, and diffusion in solution and hydrogel electrolytes is shown. In (b) Organ-specific device platforms are illustrated. Schematics (i) and (ii) show hydrogel electrochemical cell-generated H₂ protecting against ischemia-reperfusion (I/R) injury in cardiomyocytes (CMs), by neutralizing reactive oxygen species (ROS). The image (iii) shows a membrane electrode assembly-hydrogel (MEA-hydrogel) attached on a pig heart, and the schematic (iv) shows a structure of the MEA-hydrogel device and a HER process at the MEA-hydrogel device. The schematic (v) shows an H-Pad for protection against pressure ulcer injuries that integrates the MEA-hydrogel with a flexible and wireless printed circuit board (PCB). A system design of the flexible PCB that supplies constant current to the MEA device is shown at (vi). (vii) (viii) A portable H-Pad device patch is shown at (vii) and (viii), comprising Tegaderm film, MEA-hydrogel, polyimide film, battery, flexible PCB and 3D printed ring.

FIG. 3 (a)-(k) illustrates a characterization of a Ti—Pt electrode in solution and hydrogel electrolytes. Graph (a) shows a linear sweep voltammetry (LSV) of the Ti—Pt electrode in a Na₂SO₄ solution and hydrogels. Graph (b) shows electrochemical impedance spectroscopy (EIS) of the Ti—Pt electrode at −600 mV (vs. RHE) from 100 kHz to 0.1 Hz. Graph (c) shows an Electrochemical Impedance Time Spectroscopy (EITS) of the Ti—Pt electrode at −600 mV (vs. RHE) and 10 kHz. Graph (d) shows the results of a Chronopotentiometry (CP) test of reaction kinetics of the Ti—Pt electrode, −4.4 mA/cm² current density for 5 min. Images (e) shows the structure of bubbles and Ti—Pt wires in different electrolytes, using a −4.4 mA/cm² current density for 5 min. Graph (f) plots a distance from the electrode of the distalmost bubbles in various hydrogels (independent experiment n=4). Graph (g) shows H₂ leakage from Na₂SO₄ solution and hydrogels into the air, using a −0.5 mA charge for 5 min (independent experiment n=4). Image (h) shows micro-computed tomography (micro-CT) results showing H₂ trapping in the 3% hydrogel compared with solution electrolyte. Graph (i) shows H₂ diffusion with solution or hydrogel coating on a Ti mesh, using a −5 mA charge for 6 min. Graph (j) shows a concentration of dissolved H₂ with different charging currents for 6 min (independent experiment n=3). Plots (k) show H₂ diffusion modeling at 0 h, 1 h, 12 h, and 24 h with 9.33 mol H₂ gas trapped inside the hydrogel (upper panel) and heat map of H₂ profile from 0 h to 24 h at 1 cm above Tegaderm film (lower panel). 0 mm marks the Tegaderm film. Data presented as mean±s.d. P-values (shown in (f), (g)) are determined by ordinary one-way analysis of variance (ANOVA) with Tukey's multiple comparisons test.

FIG. 4 (a)-(l) illustrates a characterization of H₂ delivery via hydrogel electrochemical cell mitigating oxidative damage in cell-free and in vitro cellular environments. Graphs (a) and (b) show Hydroxyl radical (•OH) levels following a Fenton reaction in a cell-free system, with and without H₂ pre-treatment. Graph (a) shows fluorescence intensity fold changes and graph (b) shows quantification of fluorescence intensity fold changes (independent experiment n=4). Fluorescence levels measured using hydroxyl phenyl fluorescein. H₂+H₂O₂ group underwent a 30 min H₂ pre-treatment using the Ti-mesh hydrogel device prior to addition of H₂O₂. Panel (c) is a schematic of Ti mesh-hydrogel electrochemical cells producing H₂ to scavenge ROS generated by the copper-based Fenton reaction in cardiomyocytes (CMs). Panel (d) illustrates a mechanism of the copper-based Fenton reaction, which converts cellular H₂O₂ into •OH, inducing oxidative stress. Panel (e) shows CMs stained with CellROX™ demonstrate that H₂ treatment reduces the levels of cellular ROS induced by the copper-based Fenton reaction. Graph (f) shows a quantification of CellROX™ fluorescence intensity in (Cu+Vc)-treated CMs with or without H₂ pre-treatment (independent experiment n=4). Graph (g) illustrates a viability of CMs treated with or without Cu+Vc and H₂, assessed by propidium iodide (PI) and Hoechst staining. Cell viability was quantified from staining images (independent experiment n=5). Panel (h) side scatter area (SSC-A) flow cytometry analysis of PI staining in CMs treated with or without Cu+Vc and H₂. Panel (i) shows flow cytometry histograms of PI staining in CMs treated with or without Cu+Vc and H₂, corresponding to data in (h). Panel (j) shows NLRP3 immunofluorescence staining in CMs treated with or without Cu+Vc and H₂, with the right portion of the panel presenting quantitative fluorescence analysis of NLRP3 expression (n=36). Panel (k) shows Caspase-1 immunofluorescence staining in CMs treated with or without Cu+Vc and H₂, with the right panel presenting quantitative fluorescence analysis of Caspase-1 expression (n=36). In (j) and (k), the center line represents the median; the box dash lines indicate the upper (75th percentile) and lower (25th percentile) quartiles. Schematic diagram (l) illustrates the influence of H₂ treatment on inflammasome expression and Caspase-1 activity, highlighting its impact on cell inflammation and viability. Data presented as mean±s.d. P-values in (g), (j), and (k) are determined by two-sample t-test, two-tailed. P-values in (b), and (f) are determined by ordinary one-way ANOVA with Tukey's multiple comparisons test.

FIG. 5 (a)-(i) illustrates H₂ delivery via MEA-hydrogel protects ex vivo heart tissue and function against I/R injury. Panel (a) shows a schematic and photo of the MEA-hydrogel device and experimental setup for H₂ production to treat an isolated ischemic heart. Graph (b) shows an LSV of three MEA samples (without Pt or IrO₂ electrodeposition, immersed in Na₂SO₄ solution, coated with 3% PVA hydrogel) from 1 V to 3 V, plotting the apparent current density against the voltage across the cathode and anode. Image (c) illustrates an isolated rat heart experimental setup and protocols. For H₂ treatment, isolated rat hearts were immersed in 37° C. Tyrode's buffer to maintain physiological temperature and placed on Tegaderm film. MEA-hydrogel device was placed underneath the Tegaderm film and charged at a current of 5 mA for 6 min at the beginning of ischemia. Panel (d) shows representative photographs of triphenyltetrazolium chloride (TTC)-stained heart sections. White area indicates the size of infarcted area. Graph (e) illustrates a quantification of infarction size in each group (independent experiment n=5). Data presented as mean±s.d. P-values are determined by ordinary one-way ANOVA with Tukey's multiple comparisons test. Graph (f) shows left ventricular pressure (LVP) measurements in isolated rat hearts following the start of reperfusion, with or without H₂ treatment (independent experiment n=3). Graphs (g) are a spectrogram of the LVP signal shown in panel f, highlighted by the dashed-line box. Image (h) shows a multichannel electrode array that records the isolated rat heart electrocardiogram (ECG). Panel (i) includes graphs showing LVP, ECG, and ECG propagation delays recorded from sham hearts, ischemia/reperfusion (I/R) hearts, and H₂-treated I/R hearts (independent experiment n=3).

FIG. 6 (a)-(o) illustrates H₂ delivery via a portable H-Pad bioelectronic device for in vivo treatment of I/R skin pressure ulcers. (a) is a functional block diagram of the major PCB components. (b) shows elements on the PCB. (c) shows digital images showing how ischemia, reperfusion and H₂ therapy are performed. (d) illustrates an H-Pad system to treat skin pressure ulcers, showing, from bottom to top: Tegaderm film, MEA-hydrogel device, polyimide (PI) film, PCB. (e) shows Representative images of dorsal skin pressure ulcers at different time points. (f) shows thermal images of H₂-treated and control groups on day 7. (g) illustrates minimal temperature of the ulcer site on day 7 in H₂-treated and control groups (independent experiment n=5). (h) shows a comparison of wound area on day 7 in H₂-treated and control groups (independent experiment n=5). (i) shows representative H&E staining images showing thicker dermis thickness and less edema on day 7 with H₂ treatment. (j) shows dermal thickness of the dorsal ulcer site on day 7 in H₂-treated and control groups (independent experiment n=5). (k) illustrates a comparison of the inflammation lesion area of the dorsal ulcer site on day 7 in H₂-treated groups and control groups (independent experiment n=5). (1) illustrates CD4 histochemical staining in ulcer tissue after H₂ treatment shows a modulated and mild immune response. (m) illustrates a number of CD4-positive cells on day 7 in H₂-treated and control groups (independent experiment n=5). (n) illustrates CD31 histochemical staining in ulcer tissue after H₂ treatment shows enhanced neovascularization. (o) illustrates a number of blood vessels on day 7 in H₂-treated and control groups (independent experiment n=5). Data presented as mean±s.d. P-values in (g), (h), (j), (k), (m), (o) are determined by two-sample t-test, two-tailed.

FIG. 7 (a)-(e) illustrates material characterization and electrochemical performance of Ti—Pt in 0.1 M Na₂SO₄ solution. (a) show SEM-EDX images of Ti—Pt showing Pt nanoparticle on Ti. (b) is a Linear sweep voltammetry (LSV) of Ti wire with or without Pt catalyst. (c) shows Impedance of Ti—Pt and Ti electrode with −0.6 V voltage (vs. RHE). (d) is a Tafel plot of electrodes with iR correction. (e) is a graph showing electrical stability of Ti—Pt under −26.4 mA/cm² constant current for hydrogen evolution.

FIGS. 8 (a) and (b) illustrate the results of mechanical compression test on 4 mm hydrogels with varying polymer concentrations. (a) show a stress-strain plot to 20% strain, with a 0.0168 mm/s compression rate. (b) is an image of a mechanical compression test.

FIG. 9 is a graph showing frequency sweep results of hydrogels with varying PVA polymer contents at 1% oscillation strain.

FIGS. 10 (a) and (b) illustrate creep test results of hydrogels with different PVA polymer contents under 5 Pa constant stress. (a) is a graph showing stress change with time. (b) is a graph showing the strain response of difference hydrogels.

FIG. 11 (a)-(e) illustrates test results for mechanical simulations for bubble growth. (a) is a schematic diagram showing hydrogen generated from the electrode is trapped in a hydrogel. (b) is a schematic diagram of finite element modelling. (c) The debonding length as a function of the number of hydrogen (NKT). The contour plots of deformation in X2 direction and bubble profile are shown in the (d) 3% hydrogel and (e) 6% hydrogel at the same level of NKT (PV), with 240 μm and 360 μm bonded length, respectively.

FIGS. 12 (a) and (b) are Bode plots of electrochemical impedance spectroscopy of Ti—Pt under different electrolytes with −600 mV potential vs. RHE. (a) is a graph showing a plot of impedance with frequency. (b) is a graph showing a plot of phase angles with frequency.

FIGS. 13 (a) and (b) illustrate results from Electrochemical impedance time spectroscopy (EITS) of Ti—Pt in different electrolytes with −600 mV potential vs. RHE. (a) is a graph showing a plot of real impedance with time. (b) is a graph showing a plot of minus imaginary impedance with time.

FIG. 14 (a)-(e) illustrates a video analysis with two on the gas-hydrogel boundary and one inside the hydrogel. −4.4 mA/cm² of constant current was applied to Ti—Pt wire in various hydrogel electrolytes for 5 min and then observe for 2 min. (a), (c), and (e) show the three points for various electrolytes in the video. (b), (d), and (f) show the gray value change of those three points in (a), (c) and (e), respectively.

FIG. 15 is a schematic diagram showing interfacial breaking, which causes gas leaking for connected gas channel behavior in 6% and 10% hydrogel. When kept charging, the gas channel expands and pushes the Ti-hydrogel interface, causing it to break at a built pressure and opening a room for channel gas to leak into the air.

FIG. 16 is an image showing hydrogen bubbles in a 3% hydrogel, Ti wire-air interface after −4.4 mA/cm² constant current charging for 5 min.

FIGS. 17 (a) and (b) illustrate a gas leaking experiment. (a) is a schematic diagram showing gas leaking experimental setup with two electrodes: Ti—IrO₂ and Ti—Pt inserted into a closed vial with rubber septum into 1 mL liquid or gel. 0.5 mA charge was given to these two electrodes with Ti—Pt as the cathode and Ti—IrO₂ as the anode to produce gas for 5 min. After production, gas in the air was sampled for GC with a thermal conductivity detector (TCD) to detect gas leaking. (b) is a graph showing the calibration curve of hydrogen gas for GC with TCD detector.

FIG. 18 is a graph showing TCD signals of the gas leaking experiment described in FIG. 17 for solution and various hydrogels.

FIGS. 19 (a) and (b) illustrate an amount of leaked gas for 2% and 3% hydrogel samples. The 2% hydrogel sample is liquid without mechanical strength, therefore cannot trap hydrogen gas. (a) is a graph showing the TCD signal of the gas leaking experiment for 2% and 3% hydrogels. (b) illustrates the statistical result of the gas leaking experiment (independent experiment n=4). P-value is determined by two-sample t-test, two-tailed.

FIGS. 20 (a) and (b) illustrate a gas diffusion test. (a) is a schematic diagram showing a setup for the experimental and simulation gas diffusion test, with a Tegaderm container sitting onto Ti mesh-hydrogel device and a dissolved H₂ sensor inside the Tegaderm container. (b) is a graph showing H₂ diffusion simulation results, revealing how the dissolved hydrogen concentration on Tegaderm film changes over time with different amount of initially trapped H₂. 9.33 mol H₂ corresponds to the H₂ produced by −5 mA 6 min water splitting.

FIG. 21 (a)-(c) shows another gas diffusion test, showing the lack of electricity or electrolyte leakage through Tegaderm film under a direct current (DC) model. (a) is a schematic diagram showing an experimental setup with Ti mesh-hydrogel on one side of Tegaderm film and Pt wire on the other side of the Tegaderm film. Ti mesh is in 3% hydrogel and Pt wire is in 0.1 M Na₂SO₄ solution. (b) is a graph showing LSV from 0 V to 4 V. Oscillating and very small current indicates no electricity passing through Tegaderm film as it is not liquid permeable. (c) is a graph showing impedance spectroscopy at 2 V between these two electrodes with 25 mV amplitude. The logarithm of impedance is linear with the logarithm of frequency, indicating a full capacitor behavior.

FIG. 22 shows images of Cardiomyocyte cell CellROX™ fluorescent probe staining with and without Cu+100 μM Vc or H₂ treatment.

FIG. 23 illustrates Cardiomyocyte cell H₂DCFDA fluorescent probe staining with or without Cu+100 μM Vc or H₂ treatment. (a) shows fluorescence images under different treatment conditions. (b) is a graph showing statistical results of the fluorescence intensity of different treatment groups over the sham group without any treatment (independent experiment n=5). P-value is determined by ordinary one-way ANOVA with Tukey's multiple comparisons test.

FIG. 24 illustrates cell H₂DCFDA fluorescent probe staining with or without Cu+150 μM Vc or H₂ treatment. (a) shows fluorescence images under different treatment conditions. (b) is a graph showing statistical results of the fluorescence intensity of different treatment groups over the sham group without any treatment (independent experiment n=5). P-value is determined by ordinary one-way ANOVA with Tukey's multiple comparisons test.

FIG. 25 illustrates Cardiomyocyte cell live and dead staining with and without Cu+100 μM Vc or H₂ treatment.

FIG. 26 illustrates HaCaT cell live and dead staining with and without Cu+150 M Vc or H₂ treatment.

FIG. 27 is a graph showing statistical results of HaCaT cell death ratio with or without hydrogen treatment (independent experiment n=5). P-value is determined by two-sample t-test, two tailed.

FIG. 28 illustrates an influence of H₂ treatment on NLRP3 expression in CMs under oxidative stress. NLRP3 immunofluorescence (IF) staining of cardiomyocytes cultured on Tegaderm film with and without Cu+Vc or H₂ treatment.

FIG. 29 illustrates an influence of H₂ treatment on Caspase-1 expression in CMs under oxidative stress. Caspase-1 IF staining of cardiomyocytes cultured on Tegaderm film with or without Cu+Vc or H₂ treatment.

FIG. 30 is a diagram showing fabrication steps of membrane electrode assembly-hydrogel (MEA-hydrogel) device.

FIG. 31 (a)-(e) illustrates material characterization and electrochemical performance of Ti wire after IrO₂ electrodeposition in 0.1 M Na₂SO₄ solution. (a) shows SEM-EDX images of Ti—IrO₂ showing IrO₂ catalyst layer on Ti. (b) is a graph showing linear sweep voltammetry (LSV) of Ti wire with or without IrO₂ catalyst. (c) is a graph showing impedance of Ti—IrO₂ and Ti electrode with 1.8 V voltage (vs. RHE). (d) is a Tafel plot of electrodes with iR correction. (e) is a graph showing electrical stability of Ti—IrO₂ under 26.4 mA/cm² constant current for oxygen evolution.

FIGS. 32 (a) and (b) illustrates electrochemical impedance spectroscopy Bode plots at 2 V across two electrodes of MEA. 2 V is before the initiation of H₂ and O₂ production. Three samples were compared: (1) MEA without IrO₂ and Pt catalyst, denoting as MEA w/o catalyst; (2) MEA in solution; (3) MEA with hydrogel coating, denoting as MEA-hydrogel. (a) is a Bode plot of impedance versus frequency. (b) is a Bode plot of phase shift versus frequency.

FIG. 33 illustrates an H₂ diffusion simulation with a 3D-scanned rat heart model inside Tegaderm chamber at different times (0 min, 10 min, 20 min, 30 min). The rat heart is in contact with an MEA-hydrogel device, shown at the bottom of the figure. Greater H₂ exposure is observed on tissue side close to the device, shown at the bottom of the figure, and in the left ventricle and left atrium site.

FIG. 34 illustrates command of electronics of the MEA-hydrogel device via a wireless connection over a 96.9 Ohm resistor. (a) is a graph showing commanded current versus real current. (b) is a graph showing measured voltage versus real voltage.

FIG. 35 (a)-(e) illustrates images of the electronics of a MEA-hydrogel device, according to some embodiments. (a) & (b) & (c) are images of a flexible printed circuit board (PCB). (d) & (e) are images of the flexible PCB attached with a battery.

FIG. 36 is a schematic diagram of a design of electronics of the flexible PCB shown in FIG. 35.

FIG. 37 illustrates and induce and treatment timeline of a pressure ulcer, using a MEA-hydrogel device, according to some embodiments.

FIGS. 38 (a) and (b) illustrates a wireless user interface for an MEA-hydrogel device. (a) shows the user interface with 0 mA commanded current. (b) shows the user interface with 5 mA commanded current.

FIG. 39 is a graph showing average temperature of wound area on mice ulcer skin (independent experiment n=5). P-value is determined by two-sample t-test, two-tailed.

FIG. 40 illustrates a wound opening and inflammation lesions in representative zoomed-out H&E staining images. The dashed area represents inflammation lesion, and the arrow represents the length of skin without epidermis layer.

FIG. 41 is a graph showing a length of skin without an epidermis layer in control group and H₂ treated group (independent experiment n=5). P-value is determined by two-sample t-test, two-tailed.

FIG. 42 illustrates stained samples, showing a blue color channel of immunohistochemical staining with CD4 (left panel) and CD31 (right panel). Black dots represent positive cells.

FIG. 43 is a series of graphs, showing a gating strategy for flow cytometry experiments. This strategy is applied for CMs apoptosis panel.

FIG. 44 is a table showing electrical impedance spectrum simulation results of Ti—Pt in different electrolytes.

### DETAILED DESCRIPTION

In this disclosure, unless otherwise specified and/or unless the particular context clearly dictates otherwise, the terms “a” or “an” mean at least one, and the term “the” means the at least one.

Also as used herein, the terms “about,” “substantially,” and “approximately,” when used to modify numeric value or numeric range, indicate that reasonable deviations from the value or range, typically 5% or 10% above and 5% or 10% below the value or range, remain within the intended meaning of the recited value or range. The term “about” can also be used to mean a value on the same order of magnitude as the recited value. As a default the terms “about,” “substantially,” and “approximately” are inclusive of the endpoints of a relevant range, but disclosure of ranges exclusive to the endpoints is also intended.

In some examples, the present disclosure provides systems and methods for localized gas generation and delivery using hydrogels. For example, electrodes can be in contact with (e.g., embedded in) hydrogels, and electric signals can be provided to the electrodes to stimulate an electro-chemical reaction within the hydrogel, which can generate one or more gases within the hydrogel. According to some examples of the present disclosure, hydrogels for use in systems for localized gas delivery can have physical properties facilitating the trapping and controlled diffusion of gases generated within the hydrogel.

In some examples, devices, according to the present disclosure, can include a portable hydrogel electrochemical device for skin disease treatment, which is a system designed to generate, store, and deliver molecular hydrogen (H₂) locally and sustainably to treat skin diseases, particularly those associated with oxidative stress, such as pressure ulcers. The device integrates a hydrogel matrix embedded with a membrane electrode assembly (MEA) comprising two mesh electrodes (Ti—Pt and Ti—IrO₂) and a proton exchange membrane (PEM). This setup facilitates the hydrogen evolution reaction (HER) directly within the hydrogel, enabling localized and controlled H₂ production. The hydrogel matrix not only supports electrochemical reactions but also traps and regulates the diffusion of H₂, ensuring efficient delivery to the affected skin tissue. The system is powered by a compact, wireless electronic circuit that allows users to control the timing and amount of H₂ generated.

The technology addresses the limitations of existing hydrogen therapies by providing a portable and localized solution for H₂ delivery. Specifically, it can generate H₂, store or trap H₂, and controllably deliver H₂, enhancing healing and mitigating damage.

This technology has an ability to overcome the inefficiencies of conventional hydrogen delivery methods, such as gas inhalation or hydrogen-enriched water, which suffer from low bioavailability and lack of target specificity. In some embodiments, benefits can include localized therapy, sustained H₂ delivery, being portable and user-friendly, having broad application in treating conditions involving oxidative stress and inflammation, and translational potential for scalable solutions for widespread clinical adoption.

Existing technologies in hydrogen therapy include gas inhalation systems and hydrogen-enriched water. However, these approaches often lack precision, are systemic rather than localized, and suffer from rapid H₂ leakage, limiting their therapeutic efficiency. Additionally, electrochemical methods for hydrogen production, including water splitting devices and membrane electrode assemblies (MEA), are available. However, existing implementations are often designed for industrial or large-scale applications and do not integrate a biocompatible hydrogel matrix for localized and sustained H₂ delivery in therapeutic contexts. Hydrogels are also used in existing technologies for drug delivery and wound healing due to their biocompatibility and ability to retain moisture. However, existing technologies often utilize hydrogels to perform passive drug diffusion rather than active gas generation and sustained therapeutic delivery. Moreover, current technology allows for a particular hydrogel selection, as the hydrogel can be selected to control a rate of gas diffusion associated with that particular hydrogel to match a predetermined, desired rate of gas diffusion. This is accomplished by tuning the water content and polymer composition of the hydrogel to fine tune the rate of gas diffusion.

Existing hydrogen therapy methods, such as gas inhalation or hydrogen-enriched water, deliver H₂ systemically, resulting in inefficient and non-specific treatment with significant H₂ loss due to rapid diffusion and leakage. In an embodiment of the portable hydrogel electrochemical device for skin disease treatment, the hydrogel electrochemical device enables localized H₂ delivery directly to the affected area (e.g., skin or tissue), minimizing waste and maximizing therapeutic efficiency. The hydrogel matrix traps H₂ as it is generated, allowing for controlled and sustained release, which significantly extends the therapeutic window compared to conventional methods.

Existing hydrogen production systems, such as industrial electrochemical cells or large-scale MEA setups, are bulky, require harsh conditions, and lack biocompatibility, making them unsuitable for therapeutic applications. In an embodiment of the portable hydrogel electrochemical device for skin disease treatment, the device integrates a biocompatible hydrogel with a miniaturized membrane electrode assembly (MEA), enabling hydrogen generation under mild conditions suitable for biological systems. Its compact, wireless design allows for portability and easy clinical or at-home use, addressing a critical gap in existing applications of hydrogen therapy for medical applications.

While hydrogels have been used in drug delivery, existing methods typically rely on passive diffusion and do not actively generate therapeutic gases. Similarly, conventional hydrogen generation systems are designed for instantaneous gas production without integrated storage or diffusion mechanisms. In an embodiment of the portable hydrogel electrochemical device for skin disease treatment, the technology combines hydrogen generation via electrochemical water splitting with the hydrogel's ability to store and control the diffusion of H₂. This integrated approach allows for precise control of hydrogen release rates, improving therapeutic outcomes and ensuring better safety by avoiding burst releases.

Existing skin treatment technologies, such as wound dressings and ROS scavengers, primarily use passive materials or rely on external chemical or biological methods to mitigate oxidative stress. In an embodiment of the portable hydrogel electrochemical device for skin disease treatment, the hydrogel electrochemical device is versatile, as it not only treats oxidative stress-related skin conditions but can also be adapted for other ROS-mediated injuries (e.g., ischemia-reperfusion injury in organs). The ability to adjust the current and electrode composition makes it a platform technology for various gas-based therapies or drug delivery therapies, extending its potential applications beyond skin treatment.

Existing hydrogen delivery systems lack precise control over gas production and release, often leading to inefficient treatment or, in some cases, safety concerns when hydrogen is delivered systemically or in high concentrations. In an embodiment of the portable hydrogel electrochemical device for skin disease treatment, the precise control over hydrogen production enabled by the MEA and wireless electronics enhances efficiency and safety by maintaining therapeutic concentrations at the target site without systemic exposure. This also avoids flammability risks associated with mixed hydrogen-oxygen environments.

The disclosed technology bridges significant gaps in existing hydrogen delivery and ROS treatment systems by integrating localized hydrogen therapy, sustained gas delivery, and biocompatible design into a compact and portable device. These improvements make it a transformative advancement over existing technology, with significant potential for clinical and home-based therapeutic applications.

Current hydrogen therapy systems utilizing a hydrogel-covered electrochemical system can be seamlessly integrated into everyday bedding products such as pillows, mattresses, and bed covers, providing continuous hydrogen delivery to the human body during rest. Notably, the body regions in direct contact with beddings—such as the back, shoulders, hips, and heels—are subjected to prolonged pressure, making them particularly vulnerable to pressure ulcers (bedsores) due to restricted blood flow and increased oxidative stress. By embedding the hydrogel-based hydrogen generation device within bedding materials—either through direct insertion, lamination, or sewing into fabric layers—localized H₂ therapy can be applied continuously and non-invasively to high-risk areas. This ensures sustained antioxidant effects, mitigating reactive oxygen species (ROS) accumulation and reducing tissue damage caused by ischemia-reperfusion cycles. The embodiments of the current technologies that incorporate integration of this portable, wireless, and self-regenerating hydrogen delivery system includes long-term bedsore prevention and skin health maintenance in bedridden patients, elderly individuals, and those with limited mobility.

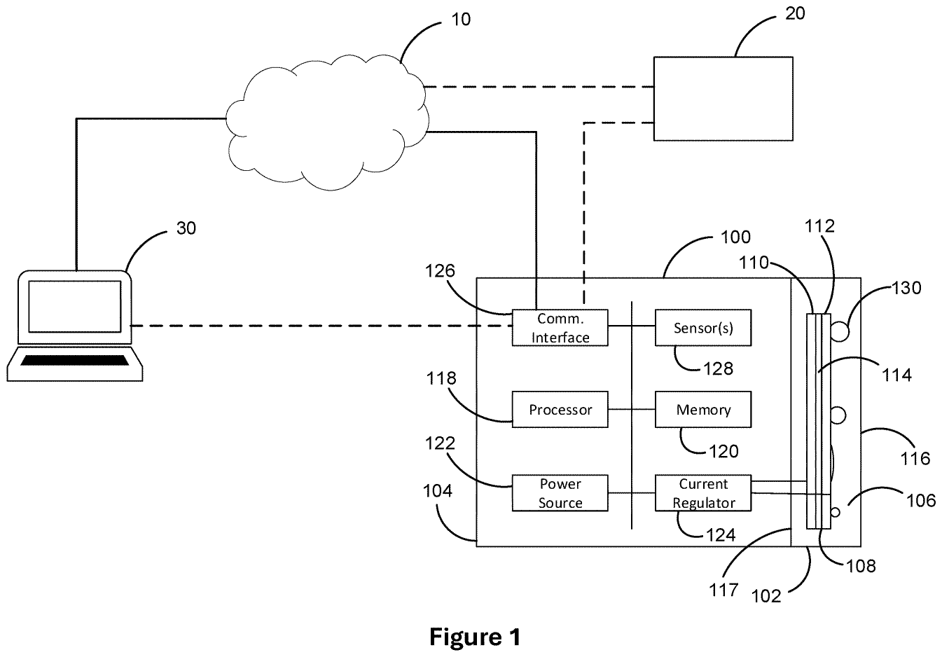

FIG. 1 illustrates an example hydrogel device 100 for generating and diffusing gas (e.g., molecular hydrogen, oxygen, nitrogen, etc.), according to some examples of the present disclosure. The hydrogel device 100 includes an electrochemical cell 102 and an electronics portion 104.

The electrochemical cell 102 can include a hydrogel 106, and a membrane electrode assembly (MEA) 108. In some examples, the hydrogel 106 can be a polyvinyl alcohol (PVA) hydrogel. In some examples, the hydrogel 106 can have a polymer concentration of up to about 3% PVA in a 0.1 M Na₂SO₄ solution. In some examples, the hydrogel 106 can have a concentration of up to about 6% or about 10% PVA in a 0.1 M Na₂SO₄ solution. In some examples, the hydrogel 106 can have a concentration of between 3% and 10% PVA in a solution. In some examples, the hydrogel 106 can be synthesized using a freeze-thaw method to physically crosslink the polymer.

In the illustrated example, the MEA 108 is embedded in the hydrogel 106. The MEA includes electrodes 110, 112, which are configured to receive electrical signals from the electronics portion 104, as described further below. In the illustrated example, the electrode 110 is a cathode, and the electrode 112 is an anode. In some examples, the electrodes 110, 112 are mesh electrodes. In some examples, one or both of the electrodes 110, 112 comprises a titanium-platinum alloy (Ti—Pt). In some examples, one or both of the electrodes 110, 112 comprises an iridium-titanium oxide (Ti—IrO₂). In some examples, one of the electrodes 110, 112, is a Ti—Pt mesh electrode, and the other of the electrodes 110, 112 is a Ti—IrO₂ mesh electrode. As further shown in FIG. 1, the electrochemical cell 102 further includes a proton exchange membrane (PEM) 114 disposed between the electrodes 110, 112. In some examples, the PEM 114 includes a sulfonated tetrafluoroethylene-based fluoropolymer-copolymer (e.g., a Nafion™ PEM).

In some examples, the hydrogel device 100 can be configured for use in treating tissue of a subject (e.g., a skin of a mammal or a human, walls of internal organs, etc.). For example, the hydrogel device 100 can be configured to be adhered directly to a skin portion to be treated, to facilitate local delivery of a treatment gas (e.g., molecular hydrogen (H₂)) directly too the skin portion. In this regard, the hydrogel device 100 includes an adhesive 116 at a surface of the hydrogel 106. In operation, the adhesive 116 can secure the hydrogel device 100 to a tissue (e.g., a tissue of a mammal). In some examples, the adhesive 116 comprises an adherent film. In some examples, the adhesive 116 can comprise a polyurethane film and an acrylate adhesive.

In some examples, the adhesive can be a Tegaderm™ adhesive. In some examples, the adhesive can be a silicone-adhesive film, an elastic cloth tape, a zinc oxide tape, or any known adhesives usable to secure a device to a tissue of a subject.

The electronics portion 104 includes a printed circuit board (PCB). In some examples, a PCB of the electronics portion 104 can be a flexible PCB. In some examples, the electronics portion can include a processor 118, a memory 120, a power source 122, a current regulator 124, a communications interface 126, and one or more sensors 128. In some examples, the electronics portion 104 is positioned at an opposite side of the electrochemical cell 102 (e.g., the hydrogel 106) from the adhesive 116. In some examples, a film 117 is provided between the electronics portion 104 and the electrochemical cell 102. In some examples, the film 117 comprises a polyimide film.

The processor 118 can include a general-purpose processor (e.g., a microprocessor) and/or a special-purpose processor (e.g., a digital signal processor (DSP)). In some examples, the processor 118 can include a field-programmable gate array (FPGA), an application-specific integrated circuit (ASIC), or a programmable logic controller (PLC). The processor 118 can execute program instructions included in the memory 120 as discussed below.

The memory 120 can include one or more volatile, non-volatile, removable, and/or non-removable storage components, such as magnetic, optical, and/or flash storage, and/or can be integrated in whole or in part with the processor 118. Further, the memory 120 can take the form of a non-transitory computer-readable storage medium, having stored thereon program instructions (e.g., compiled or non-compiled program logic and/or machine code) that, upon execution by the processor 118, cause the hydrogel device 100 to perform one or more acts and/or functions, such as those described in this disclosure. These program instructions can define, and/or be part of, a discrete software application. In some instances, the hydrogel device 100 can execute program instructions in response to receiving an input, such as an input received via the communication interface 126. The memory 120 can also store other types of data, such as those types described in this disclosure.

The communication interface 126 can allow the hydrogel device 100 to communicate with another entity, such as another computing device, according to one or more protocols. In one example, the communication interface 126 can be a wireless interface, such as a cellular or WI-FI interface. In some examples, the communications interface 126 can communicate using a short-range wireless communication protocol, such as Bluetooth®, Bluetooth Low Energy (BLE), or an IEEE 802.15-based protocol. In another example, the communication interface 126 can be a wired interface, such as an Ethernet interface. In this disclosure, a connection can be a direct connection or an indirect connection, the latter being a connection that passes through and/or traverses one or more entities, such as a router, switch, or other network device. Likewise, in this disclosure, a transmission can be a direct transmission or an indirect transmission.

The current regulator 124 can comprise one or more circuits in communication with the processor 118 and the electrodes 110, 112. The current regulator 124 can provide electrical signals to one or both of the electrodes 110, 112 based on signals received from the processor 118. In some examples, an electrical current at the electrode 112, received via the current regulator 124, can produce molecular hydrogen H₂ at an interface between the electrode 112 and the hydrogel 106. As shown, the H₂ gas can form hydrogen bubbles 130 on the electrode 112. The current regulator 124 can thus be controlled to generate and diffuse H₂ through the hydrogel, as described in greater detail below. In some examples, the current regulator includes a digital-to-analog converter (DAC) and a Howland circuit. The DAC can provide an analog signal to the Howland circuit based on a digital signal received from the processor 118, and the Howland circuit can provide a control signal to one or more of the electrodes 110, 112 based on the analog signal.

In some examples, the power source 122 can comprise a wireless power source. For example, the power source 122 can be a battery. In an example, the power source 122 is a coin battery. In other examples, the power source 122 can comprise a photovoltaic cell, in addition to or alternatively to a battery. In some examples, the power source 122 can be a wired power source (e.g., a USB connection).

In some examples, the hydrogel device 100 further includes one or more sensors 128. In some examples, the one or more sensors 128 include a dissolved H₂ sensor. A dissolved H₂ sensor can sense an H₂ within the hydrogel and can provide a feedback to the processor 118 for use in controlling the current regulator 124. In some examples, the one or more sensors 128 includes a voltage sensor configured to provide a voltage measurement from the MEA 108 to the processor 118 or the memory 120. In other examples, the one or more sensors 128 can include a current sensor, a voltage sensor, a thermal sensor, a pressure sensor, a mechanical sensor, a position sensor (e.g., a gyroscope), or the like.

The hydrogel device 100 can be in communication with external systems via the communications interface 126, and can provide information to the external systems, or receive information or instructions from the external systems. For example, FIG. 1. further illustrates a wireless communication network 10, an external sensing system 20, and a computing device 30 (e.g., a desktop, a laptop, a mobile device, a wearable computing device, a tablet, etc.). The communications network 10 can include any known networks or combination of networks for communicating with remote systems (e.g., a short-range wireless network, a local area network (LAN), a wide area network (WAN), etc.). The hydrogel device 100 can be in communication with the computing device 30 through a direct (wired) connection, or via the communications network 10. In some examples, a user can control an operation of the hydrogel device 100 via a user interface at the remote computing device 30. In some examples, the remote sensing systems 20 can provide signals to the hydrogel device 100, and the processor 118 can generate signals to the current regulator 124 based on the signals received from the remote sensing systems 20.

Examples of methods and systems are described herein. It should be understood that the words “exemplary,” “example,” and “illustrative,” are used herein to mean “serving as an example, instance, or illustration.” Any embodiment or feature described herein as “exemplary,” “example,” or “illustrative,” is not necessarily to be construed as preferred or advantageous over other embodiments or features. Further, the exemplary embodiments described herein are not meant to be limiting. It will be readily understood that certain aspects of the disclosed systems and methods can be arranged and combined in a wide variety of different configurations.

### Examples

Hydrogen (H₂) gas is integral to numerous applications, including fuel cells¹, industrial chemical synthesis², and biomedical therapies³. The small molecular size nature and flammability of H₂ poses significant challenges in storage and release for both industrial and biomedical applications. Traditional production methods, such as steam reforming or electrolysis⁴, require harsh physical or chemical conditions, bulky equipment, and storage under low temperatures or high pressures⁴. Therapeutic applications, which leverage the ability of H₂ to neutralize reactive oxygen species (ROS) for protection against ischemia-reperfusion (I/R) injury^{3,5}, are incompatible with such processes and demand mild, portable and controllable delivery systems.

Current H₂ delivery methods in clinical trials, such as gas inhalation and hydrogen-enriched water⁶, suffer from rapid leakage, difficulty in concentration control, and poor tissue specificity⁷. Alternative approaches like photocatalysis⁸, algae-bacteria systems⁹, and galvanic cells¹⁰ are promising in mice, but exhibit limited external control and inconsistent production rates under dynamic biological conditions, rendering them unsuitable for precise and sustained therapy. These challenges underscore the need for advanced electrochemical engineering approaches that leverage material engineering, interfacial kinetics, and molecular transport to enable controlled H₂ production and sustainable delivery for biomedical applications.

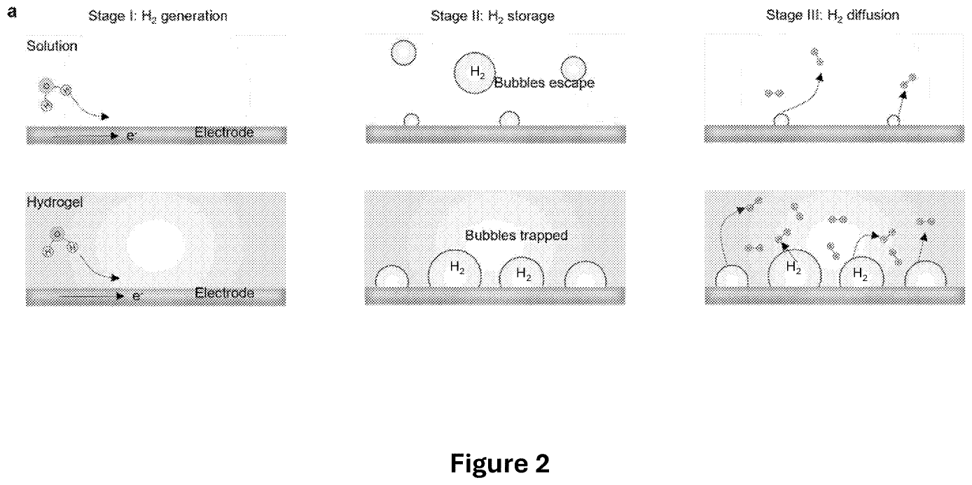

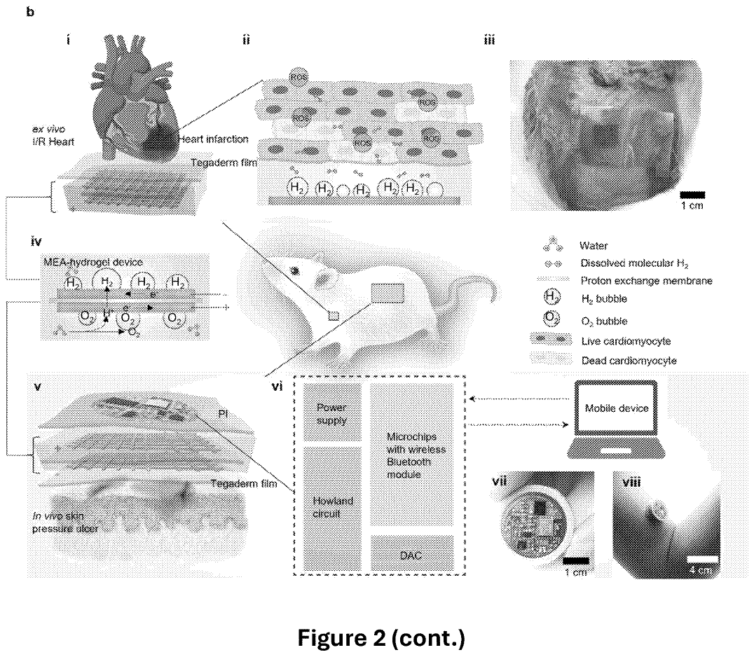

In this study, we introduce the first hydrogel electrochemical cell designed for the controlled generation, localized storage, and sustained diffusion of H₂ under mild, portable, and regulated conditions. Hydrogels—water-rich, elastic, and adaptable materials—are promising candidate for bioelectronic interfacing with biological systems¹¹ and present an underexplored system for the hydrogen evolution reaction (HER). Hydrogels, as soft materials, can be highly permeable to gases, making them ideal for gas therapy purposes^{12,13}. In the traditional liquid electrolyte-based HER, H₂ bubbles escape readily, leading to inefficient diffusion and utilization in biological systems (FIG. 2 (a), upper panel). In contrast, the three-dimensional (3D) water-polymer network of the hydrogel supports the HER while effectively and immediately trapping H₂ bubbles, for safe and sustainable diffusion and improved biointerface delivery (FIG. 2 (a), lower panel). We thoroughly study the H₂ evolution and dynamics in a pure hydrogel electrolyte system compared with liquid electrolyte system, evaluating the influence of hydrogel polymer composition on electrochemical kinetics, gas morphologies, and gas storage.

We demonstrate the efficacy of the system in neutralizing ROS and preventing oxidative damage in a cell-free system and in cardiomyocytes (CMs) and keratinocytes in vitro (FIG. 2 (b)(ii)). For tissue-hydrogel evaluation, we develop a membrane electrode assembly-hydrogel (MEA-hydrogel) device by attaching two Ti mesh electrodes on either side of a Nafion™ proton exchange membrane (PEM) and embedding them in hydrogel to create a compact and portable electrochemical cell (FIG. 2 (b)(iv)). The hydrogel, serving as a biocompatible scaffold, traps H₂ and controls its release to the tissue-device interface, enabling localized and sustained H₂ delivery in ex vivo ischemia-reperfusion (I/R) heart models (FIG. 2 (b)(i) & (iii)) and in vivo skin pressure ulcer models (FIG. 2 (b)(v)). We integrate a wireless flexible printed circuit board (PCB) to supply constant current to the MEA-hydrogel device, which we envision will allow on-demand control of H₂ production and dosage adjustment by patients or clinicians (FIG. 2 (b)(vi-viii)).

The hydrogel electrochemical cell platform offers a promising and streamlined approach to the treatment of ischemia-reperfusion (I/R) injuries. The hydrogel itself fulfills three critical functions: H₂ generation, storage, and diffusion, effectively integrating these processes into a single system. This multifunctionality not only simplifies traditional H₂-based therapies but also reduces costs while enabling a portable and wireless therapeutic solution. Beyond H₂ therapy, the platform's ability to facilitate electrochemical reactions within a hydrogel, coupled with its capacity for localized gas trapping and sustained diffusion, positions it as a versatile system for broader applications in other gas-based therapies and drug delivery research.

### Materials and Methods

### Animal Protocols

All animal procedures were approved by the Institutional Animal Care and Use Committee of the University of Chicago in protocol numbers 72378 (rat) and 72621 (mice).

### Chemicals

Ti wire (diameter 0.25 mm) and Ti mesh (80 mesh, diameter 0.13 mm) were purchased from Fisher Scientific. Iridium(IV) chloride hydrate (IrCl₄·H₂O), potassium tetrachloroplatinate(II) (K₂PtCl₄), and polyvinyl alcohol (PVA, 99%+ hydrolyzed, molecular weight (Mw) 89,000-98,000) were purchased from Sigma-Aldrich. Tegaderm™ Ag Alginate Silver Dressing was purchased from 3M. Anti-NLRP3 monoclonal antibody was purchased from Biotechne, and anti-Caspase-1 monoclonal antibody was purchased from Proteintech. Unless otherwise noted, all other chemicals were purchased from Sigma-Aldrich without further purification.

### Electrodeposition of Electrocatalyst on Ti Electrode

To prepare the electrodeposition solution, 0.1 M iridium chloride (IrCl₄·H₂O) was dissolved in DI water and stirred for 30 min. Then, 40 mM oxalic acid [(COOH)₂·2H₂O] and 100 mM hydrogen peroxide (H₂O₂) were added, and the mixture was homogenized for 10 min. To adjust the pH to 10.5, 340 mM potassium carbonate (34.5% K₂CO₃) was added, followed by stirring for 3 days to ensure the stability of the prepared electrodeposition solution⁴¹. Ti electrode was electrodeposited inside the solution with 0.7 V (vs. saturated Ag/AgCl electrode) for 180 s to yield the Ti—IrO₂ electrode.

For electrodeposition of Pt, a solution with 2.0 mM K₂PtCl₄ and 0.5 M HCl was prepared. The Ti electrode was electrodeposited inside the solution with −0.25 V (vs. saturated Ag/AgCl electrode) for 400 s to yield the Ti—Pt electrode.

### Preparation and Coating of Hydrogel on Electrode

3 wt %, 6 wt %, 10 wt % PVA hydrogel precursor solution was prepared by dissolving 3 g, 6 g, 10 g PVA powder in 0.1 M Na₂SO₄ solution, respectively. The mixture was stirred and heated to 80° C. to ensure homogeneity, and then cooled to room temperature. The hydrogel precursor solution was poured onto the electrode and frozen in a −20° C. freezer for 16 h, after which time it was placed at room temperature to thaw for 8 h to achieve a hydrogel coating of 1.3 mm thickness on electrode.

### Electrochemistry Tests of Ti—Pt Electrode in Solution and Hydrogel

Linear sweep voltammetry (LSV), electrochemical impedance spectroscopy (EIS), electrochemical impedance time spectroscopy (EITS), and chronopotentiometry (CP) were conducted with the aid of a potentiostat (Corrtest electrochemical workstation). For these experiments, a Pt wire served as counter electrode, while an Ag/AgCl electrode immersed in saturated KCl acted as reference electrode. The working electrodes were Ti—Pt electrodes. Electrolytes used were 0.1 M Na₂SO₄ solution or 3%, 6%, 10% hydrogel. For LSV measurement, the sweep rate was set at 10 mV/s. EIS measurements were taken in potentiostatic mode at −1.2 V (vs. saturated Ag/AgCl electrode), applying a sinusoidal voltage with an amplitude of 25 mV and frequency from 100 kHz to 0.1 Hz. EITS measurements were taken in potentiostatic mode at −1.2 V (vs. saturated Ag/AgCl electrode), applying a sinusoidal voltage with an amplitude of 25 mV and frequency of 10 kHz. CP measurements were set at −0.5 mA for 0.114 cm² electrode surface area for 5 min. Current density was calculated to be−4.4 mA/cm².

The calculation of E(vs. RHE) is based on the following equation:

E(vs. RHE)=E(vs. Ag/AgCl)+E(Ag/AgCl)+pH×0.059 (Equation 1)

### Quantification of H₂ Leaking, Gas Chromatography (GC) and Micro-Computed Tomography (Micro-CT) Analysis

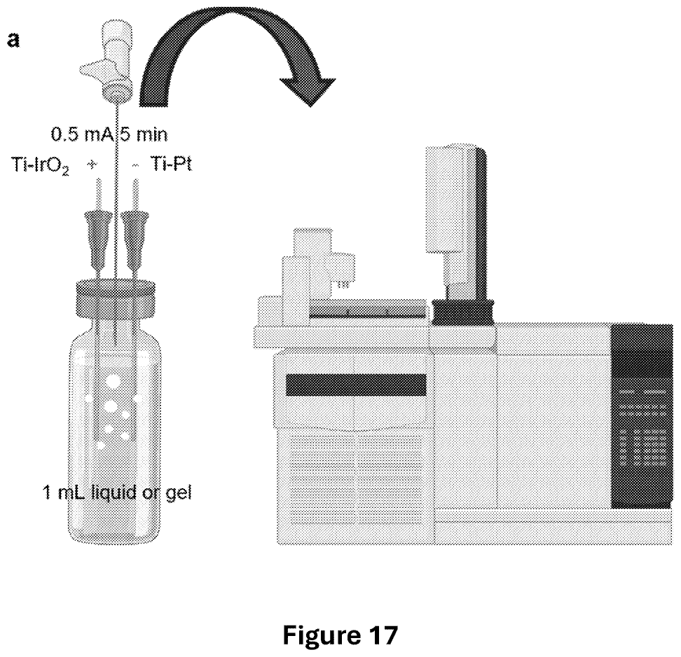

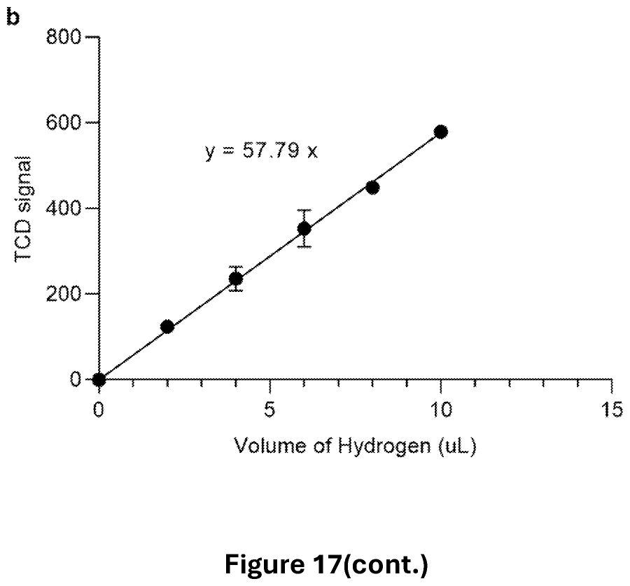

As shown in FIG. 17, two hollow needles were inserted into a 2 mL vial through silicone septa. Two electrodes (Ti—Pt and Ti—IrO₂) were inserted into the hollow core of the needles and any gaps were sealed with epoxy. 1 mL 0.1 M Na₂SO₄ solution or hydrogel precursor solution was injected into the vial. The vials underwent a 16 h freeze and 8 h thaw, after which the system was sealed tightly. 0.5 mA constant current was supplied to the two electrodes with Ti—Pt as the cathode and Ti—IrO₂ as the anode for 5 min to electrolyze water. The air inside the vial was then sampled for GC runs on the Agilent 7890B system equipped with both a flame ionization detector (FID) and a thermal conductivity detector (TCD) with N₂ flow as reference gas. Whole vials with different electrolyte (solution or hydrogel) were used for micro-CT analysis (X-Cube microCT scanner).

Soluble Hydrogen Test with or without Hydrogel Coating

One 1.8 cm×1.8 cm Ti mesh was cut, wired, and electrodeposited with Pt. The mesh was then placed onto a 1 mm glass slide in a 58 cm² petri dish and 14.8 mL Na₂SO₄ solution or 3% hydrogel precursor solution was poured in. For the Ti mesh-hydrogel electrochemical cell, a freeze/thaw was performed to achieve a 1.3 mm thick hydrogel coating on the Ti mesh electrodes. A hollow acrylic model (2.2 cm×2.2 cm×3.6 cm) was made by laser cutting. A gas permeable and liquid impermeable 3M Tegaderm™ film was attached to one side of the acrylic model and sealed with PDMS. This Tegaderm™ chamber was then placed onto the Ti mesh leaving a 1.3 mm gap between electrode and film. 10 mL Na₂SO₄ solution was added into the Tegaderm™ chamber. A 6 min −5 mA constant current charge was supplied to the Ti—Pt electrode with saturated Ag/AgCl as reference electrode and Pt wire as counter electrode. To measure how much H₂ was dissolved in the solution, a H₂ water sensor (Yewhick) was placed into the Tegaderm™ chamber, leaving a 9 mm distance from the bottom of Tegaderm™ film.

### Hydrogen Diffusion Modelling Inside Hydrogel

Hydrogen diffusion modeling used the same setup as the soluble H₂ tests. 3D diffusion simulations were performed on COMSOL. The hydrogel was modeled as a cylinder (“bubble”) with diameter 8.8 cm and height 1.3 mm. The H₂-containing bubble was modeled as a cuboid with width 1.8 cm, depth 1.8 cm, and height 0.7 mm. The solution inside the Tegaderm™ chamber was modeled as a cube with width 2.2 cm, depth 2.2 cm, and height 3.6 cm. The bubble was located inside the hydrogel at the bottom. The distance from the bottom of hydrogel to the Tegaderm™ was 1.3 mm. The height of the H₂ bubble was calculated such that the number of moles of H₂ (9.33 mol) in the bubble volume (at atmospheric pressure) was equal to the H₂ generated by the device after 5 mA charging for 6 min. Open boundary conditions were used for the boundaries of the modeling domain. ‘Thin impermeable barrier’ boundary conditions were set for the bottom of the hydrogel and the side walls of the Tegaderm™ chamber. The initial H₂ concentration in the bubble was set as 40.3 mol/m³, which is equivalent to pure H₂ concentration at atmospheric pressure. The boundaries of the model were fixed, so as the H₂ diffused away from the bubble, the concentration reduced but the size of the bubble was fixed. The diffusion coefficient of H₂ was set as: bubble and air: 0.756 cm²/s, hydrogel: 1.26×10⁻⁵ cm²/s, solution: 5.11×10⁻⁵ cm²/s. The determination of diffusion coefficient in hydrogel is based on a previous paper on gas diffusion inside PVA hydrogel¹³:

P=Pₚ(1−ε)+P_{w}ε/τ (Equation 2)

Where Pₚ and P_{w} are the permeabilities of the polymer matrix and water, respectively, F is the volume fraction of water in the hydrogel membrane and τ is the tortuousity of the water passageways, estimated to be 3.92. Since the polymer content is as low as 3% and Pₚ generally has a much lower value than P_{w}, the hydrogel membrane permeability is primarily determined by the water content.

Air-liquid interfaces were set using the ‘Partition Condition’ boundary condition. The partition coefficient value was set as 0.019 for the bubble-hydrogel interface, and 52.4 for the hydrogel-air and medium-air interfaces. These numbers were calculated using Henry's coefficient for H₂ in water.

The simulation data is plotted into heat map using customized python scripts.

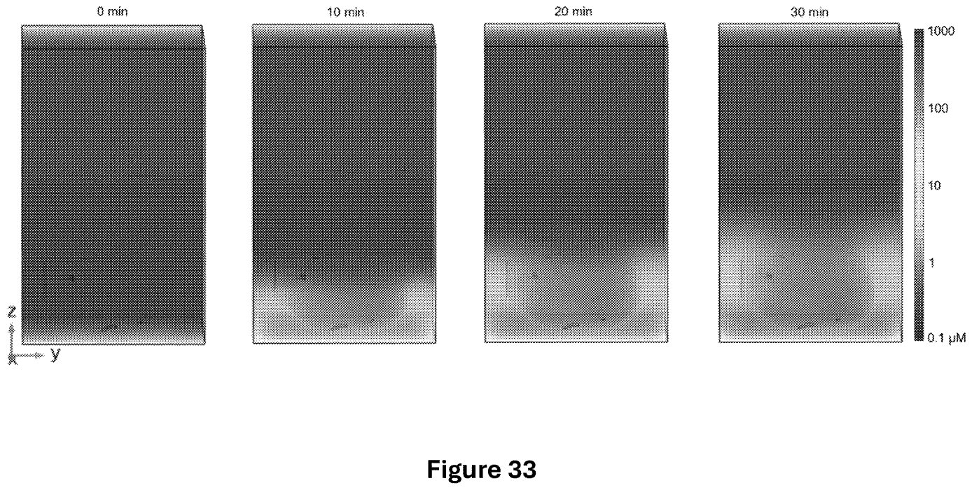

For the H₂ diffusion of a rat heart model placed within the Tegaderm™ chamber (FIG. 33), a 3D STL model of a rat heart was prepared by photogrammetry. The heart was isolated from an adult rat (400-500 g), cleaned with PBS, and fixed in 4% formaldehyde. The heart was then suspended on a needle on a small turntable, ~150 pictures were taken, and an STL model was prepared using Agisoft Metashape. The model was then post-processed on Fusion360 by removing artefacts, cropping the model, repairing holes, and remeshing to a simpler model. The STL model was then imported into COMSOL for the diffusion simulation.

### Tegaderm™ Containers for Cell-Free System ROS Verification, Cardiomyocyte and HaCaT Cell Culture

Gas permeable and liquid impermeable 3M Tegaderm™ was used to culture the cells. Acrylic was cut into pieces with an inner empty 1×1 cm square. The pieces were placed onto Tegaderm™ and PDMS precursor was used to seal the boundary between Tegaderm™ and acrylic. The assembled acrylic containers were placed in a 75° C. oven and cured for 3 h. The containers were then washed with 70% ethanol overnight to remove all PDMS monomers and catalyst residue, and then rinsed with water and IPA. Containers were treated with 350 W oxygen plasma for 180 s at 70° C. and coated with fibronectin/gelatin solution for 30 min for use. Neonatal rat CMs and HaCaT cells were cultured on the containers with 1×10⁵/cm² cell density.

Reaction of H₂ with Hydroxyl Radicals ROS in a Cell-Free System

To verify the reaction of H₂ with •OH, we adopted a previously reported Fenton reaction method³. We made a PBS solution with 0.1 mM ferrous perchlorate and 1 μM 2-[6-(4′-hydroxy)phenoxy-3H-xanthen-3-on-9-yl]benzoate (HPF) dye. 0.5 mL of the solution was added into the cell-Tegaderm™ container. For the H₂-treated group, the container was placed onto a Ti mesh-hydrogel device for a total of 30 min. A −5 mA constant current charge was supplied to the Ti mesh for 6 min with Ag/AgCl as reference electrode and Pt wire as counter electrode. The container was then placed under a fluorescence microscope to monitor fluorescence intensity. 10 μL 250 μM H₂O₂ was added into the container. The control group received H₂O₂ but no H₂ treatment. For the PBS group, 10 μL PBS instead of H₂O₂ was added into the Tegaderm™ container.

### Intracellular Fenton Reaction to Induce Hydroxyl Radicals ROS in Cells

We used the intracellular Fenton reaction as described in a previous paper³. We charged the Ti mesh-hydrogel electrochemical cell as described above with −5 mA for 6 min with saturated Ag/AgCl as reference electrode and Pt wire as counter electrode. For the H₂+Cu+Vc and H₂-only groups, the Tegaderm™ cell containers were placed onto the Ti mesh hydrogel device for constant H₂ delivery. For the H₂+Cu+Vc and Cu+Vc groups, we removed the culture media and preincubated CMs and HaCaT cells with 1 mM CuSO₄ for 30 min with corresponding cell culture media containing 10% FBS. The Cu media was removed and cells were washed once with PBS containing CaCl₂ (0.1 g/L), MgCl₂·6H₂O (0.1 g/L), glucose (1 g/L) and sodium pyruvate (0.036 g/L), and then exposed to 100 mM (for CMs) or 150 mM (for HaCaT cells) ascorbate (vitamin C) for 1 h in PBS as described above. Note that Cu²⁺ is reduced by ascorbate to Cu⁺, which catalyzes the Fenton reaction to produce •OH from H₂O₂ that is spontaneously produced in the cells.

### Membrane Electrode Assembly (MEA) Fabrication

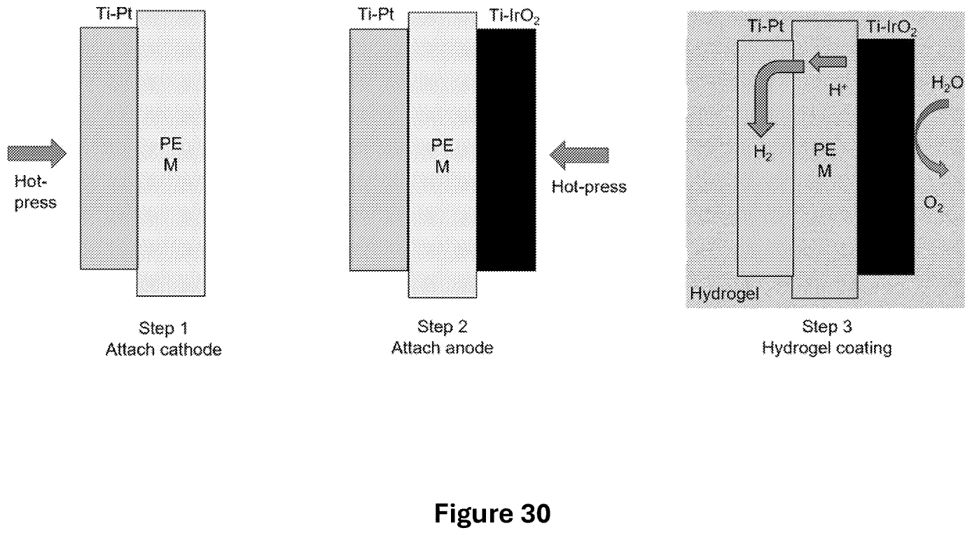

The insulation coating on the end of thin wires (A-M system) was removed and was weaved into the Ti meshes. The meshes were then electrodeposited with IrO₂ or Pt to serve as the anode or cathode, respectively. As shown in FIG. 30, the Ti—Pt mesh was then immersed into Nafion™ solution and was hot-pressed onto one side of a Nafion™ 117 membrane. Ti—IrO₂ was immersed into Nafion™ solution and was hot-pressed onto the other side to form the MEA. A 3% hydrogel precursor solution was poured onto the MEA device with the Pt side facing up to create an MEA-hydrogel device with 3% hydrogel of 1.3 mm thickness on top of the cathode side. A small amount of 3% hydrogel precursor solution will leak into the IrO₂ side and form a thin layer of hydrogel on the anodic side.

### Ex Vivo Isolated Heart Ischemia and Reperfusion Model

The rat heart isolation procedure was carried out according to our previously established protocols^{42,43}. In summary, adult male rats weighing between 400 and 500 g were treated with heparin and then anesthetized with isoflurane using a bell jar. Subsequently, the heart was removed and quickly placed into a cold HBSS solution. A cannula was inserted into the aorta to set up for Langendorff perfusion. A heated and oxygen-enriched Tyrode's solution, buffered with HEPES, was circulated through the aorta, utilizing a system that included a heating coil and a bubble trap (Radnoti). The heart was then positioned within a water-jacketed container, ensuring the temperature remained steady at 37° C. Perfusion pressure was maintained between 80 and 100 mm Hg. Monitoring of perfusion and left ventricular pressures was achieved through a BP-100 probe (iWorx) attached to the perfusion line and a water-filled balloon placed inside the left ventricle, respectively. For electrocardiogram (ECG) recordings, needle electrodes were placed on the left ventricular wall and the aorta, grounded through the cannula, and linked to a C-ISO-256 preamplifier (iWorx). All signals (perfusion, left ventricular pressure, and ECG) were amplified by 400 times using an IA-400D amplifier (iWorx) and interfaced with a computer using a Digidata 1550 digitizer with Clampex software (Molecular Devices). To induce an ischemia/reperfusion (I/R) injury model, the isolated heart was initially perfused with Tyrode's solution for 1 h. Ischemic damage was simulated by stopping the perfusion buffer flow for 30 min, during which the hearts were kept in Tyrode's solution at 37° C., followed by a 45-min reperfusion period. To protect the heart tissue from I/R injury, at the beginning of ischemia, the heart was placed into the Tegaderm™ chamber (2.2 cm×2.2 cm×3.6 cm) as previously described in hydrogen diffusion modelling with the MEA-hydrogel device underneath the chamber. The cathodic side was facing up to supply H₂ to the heart. The MEA device was charged with 5 mA for 6 min. For each different groups (sham, I/R, I/R+H₂), five rat hearts were used.

### Multichannel ECG Isochronal Maps

Fabrication of a multichannel ECG electrode was adopted from the previously reported method⁴⁴. Recordings from the 16-channel MEA on the ex vivo rat heart were analyzed by customized python scripts. Gaussian interpolation was used to enhance readability.

### Assessment of Infarct Size

Following 30 min of ischemia and 45 min of reperfusion in the Langendorff apparatus, myocardial infarct size in the isolated rat heart was assessed using triphenyltetrazolium chloride (TTC) staining. The heart was frozen in a −80° C. refrigerator and subsequently sectioned into 1 mm-thick transverse slices. The slices were incubated in 1% TTC solution at 37° C. for 30 min. After staining, images of the heart slices were captured immediately for analysis.

### In Vivo Skin Ischemia and Reperfusion Model

Ten C57BL/6 mice (8-10 weeks) mice were randomly classified into two groups (control group and H₂ group). They are anesthetized with isoflurane. The dorsal hair was shaved and the area was cleaned with 70% ethanol. The skin was gently pulled up and placed between 2 round ceramic magnetic plates (12 mm diameter and 5 mm thick) with an average weight of 2.4 g and 1000 G magnetic force (Magnetic Source) for 6 h and then removed. The resultant “pinch” procedure was designed to leave a 0.25 cm skin bridge between the two magnets. After removing the ceramic magnetic plates, for the H₂ group, the H₂ generation device, with Tegaderm™ film, MEA-hydrogel, PI film, and PCB, were placed onto one skin wound. The PCB was programmed to charge the MEA-hydrogel device with 5 mA for 6 min every 12 hours a total of three times. For control group, after removing the ceramic magnetic plates, Tegaderm™ film is placed on the skin wound. Digital images were captured on day 0, day 2, day 4, day 6 and day 7. On day 7, a thermal camera was used to capture the temperature distribution of the mouse ulcers. Skin tissue was collected for histological analysis and immunohistochemical analysis.

### Histology and Immunohistochemistry

For tissue histological analysis, H&E, CD4 and CD31 histological tissue sections were prepared by the Human Tissue Resource Center at the University of Chicago. Tissue sections of the mice dorsal skin were prepared and stained with CD4, CD31 and H&E. All tissue histological results were analyzed using the CaseViewer software (3DHISTECH). The CD4 statistic result was calculated from sampling in a random 400 μm×400 μm square under the wound. The CD31 statistic result was calculated from sampling in a random 800 μm×800 μm square under the wound.

### Data Processing and Statistics

Data analysis was performed with Python scripts using the CV2, NumPy, Matplotlib, SciPy and Pandas libraries. Plotting was performed with GraphPad Prism 9, Origin and Python Matplotlib. Statistics were calculated using GraphPad Prism 9 unless otherwise clarified. Images were processed using ImageJ software. All error bars indicate the standard deviation unless otherwise stated. Multiple t-tests and one-way analysis of variance (ANOVA) were performed for all biological data analysis. A value of P<0.05 was considered statistically significant.

### Scanning Electron Microscopy

Surface structures of various substrates and carbon membranes were characterized using scanning electron microscopy (SEM) (Merlin, Carl Zeiss). Energy-dispersive X-ray spectroscopy (EDX) was performed on the Merlin scanning electron microscope (Carl Zeiss) using an Oxford Ultim Max 100 energy-dispersive X-ray spectroscopy system. Data analysis was performed using AZTEC software (Oxford Instruments).

### Tafel Plot

iR corrected linear sweep voltammetry (LSV) were conducted with the aid of a potentiostat (Corrtest electrochemical workstation). For these experiments, a platinum wire served as the counter electrode, while an Ag/AgCl electrode immersed in saturated KCl acted as the reference electrode. The working electrodes utilized were Ti wire electrode. The sweep rate was set at 10 mV/s. The Tafel slope derived from the iR-corrected LSV curve, which was calculated according to the following equation:

η=A log(i/i0) (Equation 3)

where η, i, i0, and A represent the overpotential, current density, exchange current density, and the Tafel slope, respectively.

### Stability Tests

Chronopotentiometry (CP) were conducted with the aid of a potentiostat (Corrtest electrochemical workstation) under −26.4 mA/cm² constant current for hydrogen evolution and 26.4 mA/cm² for oxygen evolution for 12 h. For these experiments, a platinum wire served as the counter electrode, while an Ag/AgCl electrode immersed in saturated KCl acted as the reference electrode. The working electrodes utilized were Ti wire electrode coated with Pt or IrO₂ catalyst.

### Mechanical Compression Tests

Circular Hydrogel samples with 3 cm diameter and 4 mm thickness were made through freeze-thaw process. The hydrogel sample was placed between two flat acrylic surface with Tensile testing machine (Zwick-Roell zwickiLine Z0.5) for compression modulus measurement with 0.0168 mm/s compression rate to 20% strain change.

### Rheometer Frequency Sweep and Creep Tests

The rheological behaviors of hydrogel samples were evaluated on a Strain-controlled shear rheometer (TA Instruments ARES-G2) from 0.1 to 100 rad/s angular frequency at 1% oscillation strain. Creep test of PVA hydrogel were performed under 5 Pa constant stress.

### Finite Element (FE) Modelling for Bubble Morphology

Commercial software ABAQUS® was used to simulate the bubble growth in two different types of hydrogels 3% and 6%. We assume once gas was generated, it will grow symmetrically along the two ends of the electrode rod. In this way, due to the symmetry, we can simulate a bubble growing in one direction, i.e. half model. Finite Element (FE) modeling along with the cohesive zone model were employed: we introduced an initial gap between the hydrogel and electrode rod with a half-length of 20 μm, and the remaining parts are adhesive. The bonded length is 480 μm, long enough to eliminate the boundary effects. Internal pressure was applied on the inner surface of the bubble. We also implement ABAQUS® subroutine DLOAD to apply the same pressure for the new de-bonded area. In general, a 2D model was developed using the dynamic quasi-static analysis.

The Neo-Hookean was used to model hydrogels. Based on the experiment, we set the shear modulus as 39.31 Pa and bulk modulus as 15.18 Pa for 3% hydrogel; the shear modulus as 525.95 Pa and bulk modulus as 444.65 Pa for 6% hydrogel. And we set the electrode rod as the rigid body that cannot deform during bubble growth. The element type was linear plain strain with hybrid formulation (CPE4H).

As the adhesion between hydrogel and wire is so weak and hard to measure, the debonding behavior setting is based on experience. A layer of cohesive elements was embedded between the hydrogel and the electrode rod with a linear traction-separation law.

The debonding process with increasing bubble pressure is related to mixed mode fracture, therefore, we determined mixed fracture criteria with a power law 1.2. The three types of mode have the same maximum nominal stress in the traction-separation law as 200 Pa and the fracture energy 0.25·10-3 J/m². It is assumed that the interfacial cohesion energy and interface strength are the same for 3% and 6% hydrogel. In addition, a viscous damage variable of 0.0002 s was used to mitigate the convergent issue. The cohesive elements were meshed with maximum degradation of 0.98, which means once the stiffness degradation ≥0.98 for an element, that element is considered as damaged. To prevent penetration of the hydrogel coating and the substrate as compression occurs during the simulation, a node-to-surface contact with finite sliding was assigned between them. We chose the tangential behavior to be frictionless and the normal behavior to be hard contact.

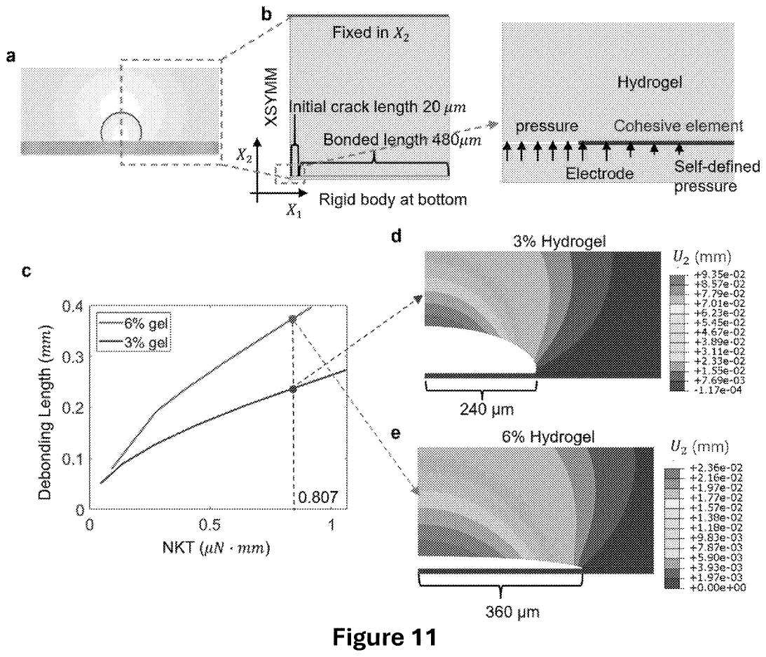

Based on the isothermal system and the ideal gas law P·V=NkT, where P is the applied pressure and V is the volume, we check the bubble profile with the same number of air molecules N generated. When P·V=0.807 μN·mm, the FIGS. 11 (c) and (d) shows the bubble profile and displacement U₂ contour of 3% and 6% hydrogel We can see for 3% hydrogel, which hydrogel is softer, it deforms more like a circle and there is less separated area than the 6% one.

### Cell Cultures

### HaCaT Culture

HaCaT cells were cultured in high glucose DMEM supplemented with 10% fetal bovine serum (FBS), 1% GlutaMAX, and 1% penicillin-streptomycin. The cells were maintained at 37° C. in a humidified incubator with 5% CO₂.

### Cardiomyocyte Culture

Hearts from neonatal rats (1-3 days old) were harvested and immediately placed in ice-cold HBSS (without Ca²⁺ and Mg²⁺). The hearts were bisected, washed three times with ice-cold HBSS to remove residual blood, and finely minced into pieces of approximately 1-2 mm in size. Tissue digestion was performed using the Pierce Primary Cardiomyocyte Isolation Kit (Thermo Fisher Scientific) according to the manufacturer's protocol. Post-digestion, the tissue was vigorously pipetted 25-35 times to achieve further dissociation. The resulting cell suspension was allowed to pre-settle for 2 hours to facilitate fibroblast adhesion to the culture dish. The supernatant containing cardiomyocytes was collected and seeded on fibronectin-coated Tegaderm™ film. Cells were initially incubated in culture medium DMEM supplemented with 10% FBS, 1% GlutaMAX, and 1% penicillin-streptomycin for 24 hours, after which the medium was replaced with a cardiomyocyte-specific medium (high glucose DMEM supplemented with 10% FBS, 1% penicillin-streptomycin, and 0.1% cardiomyocyte growth supplement).

### Immunofluorescence

Cells were fixed with 4% paraformaldehyde in PBS for 15 min at room temperature, permeabilized with 0.3% Triton X-100 in PBS for 15 min at room temperature and blocked with 5% bovine serum albumin (BSA) for 1 h at room temperature. Samples were incubated with primary antibodies in blocking buffer overnight at 4° C. Subsequently, samples were washed three times with PBS and incubated with secondary antibodies for 1 h at room temperature. The primary antibodies used were anti-NLRP3 (Biotechne, NBP2-12446) and anti-Caspase 1 (Proteintech, 22915-1-AP). Stained cells were imaged using a Nikon inverted microscope with 20× or 60× objectives, and images were analyzed using ImageJ.

### Characterization of In Vitro ROS Production

### Measure Cellular ROS by H₂DCFDA

Cellular ROS levels in HaCaT cells and CMs were quantified using 2′,7′-dichlorodihydrofluorescein diacetate (H₂DCFDA, (Abcam, ab113851)). Cells were incubated with a 20 μM H₂DCFDA solution for 45 min at 37° C. in the dark to allow for dye uptake. Following incubation, the H₂DCFDA solution was removed, and the cells were treated with or without H₂ or Cu+Vc as experimental conditions required. After treatment, the cells were harvested and seeded into 96-well microtiter plates. The H₂DCFDA fluorescence intensity, indicative of ROS levels, was immediately measured using a fluorescence plate reader (Synergy Neo HTS) at excitation/emission wavelengths of 485/535 nm.

### Measure Cellular ROS by CellROX™

To assess cellular ROS levels, cells subjected to various treatments were stained with CellROX™ Reagent (Thermo Fisher) at a final concentration of 5 μM. The staining procedure involved incubating the cells with the reagent for 30 min at 37° C. After incubation, the medium was aspirated, and the cells were washed three times with PBS to remove residual dye. Subsequently, the cells were fixed with 3.7% formaldehyde for 15 min at room temperature. To visualize the nuclei, the cells were counterstained with DAPI. Fluorescent images of the stained cells were acquired using a Nikon inverted microscope equipped with a 20× objective lens. The images were analyzed using ImageJ. Fluorescence intensity fold is calculated using sample fluorescence intensity over sham group without any treatment.

### Assessment of Cell Viability

### Hoechst 33342/PI Double Staining Assay

Cell viability was evaluated using the Hoechst 33342/PI double staining assay. Hoechst 33342 is a fluorescent dye that binds to DNA and permeates intact cell membranes, enabling the staining of nuclei in both living and dead cells. PI is a fluorescent nucleic acid dye that selectively stains cells with compromised membranes, marking all dead cells. Cells were incubated with Hoechst 33342 and PI for 10 min under appropriate culture conditions. The rate of apoptosis was calculated as the number of PI-positive cells/Hoechst 33342 positive cells×100%.

### Flow Cytometry

Following various treatments, harvest the cells, wash them in cold PBS, and centrifuge to discard the supernatant. Resuspend the cells in PBS, determine the cell density, and dilute to −1×10⁶ cells/mL. Add 1 μL 100 μg/mL PI working solution to 100 μL of the cell suspension, incubate at room temperature for 15 min, then add 400 μL PBS, mix gently, and keep on ice. Analyze the samples promptly by flow cytometry on the BD LSRFortessa™ 4-15 HTS cell analyzer (BD Biosciences). Data were exported and analyzed using the Flowjo software.

### Flexible PCB Fabrication and Implementation

### Fabrication of Flexible PCB

Custom-designed flexible printed circuit boards were manufactured by JCLPCB commercial vendor, in compliance with the ISO 9001 certificate and ISO 14001 certificate. The electronic components integrated into the circuit boards included passive elements like capacitors, inductors and resistors (Mouser Electronics). Additionally, the boards had LDO Voltage Regulators (3.3V 500 mA ULTRA LDO), Crystals (32.768 KHZ 12.5 PF SMD), Bluetooth® Modules (802.15.1 Bluetooth® Module), Switching Voltage Regulators (PFM Control Snchrns StepUpDCDC Converter), Digital to Analog Converters (DAC Sngl 8B NV DAC w/Ext Vref & I2C interface), Operational Amplifiers (Op Amps Lw Cst CMOS Hgh Spd RR Amp Triple). All circuit components were soldered using leadfree no-clean solder (Chip Quik Inc., model no. Sn96.5Ag3Cu0.5 (96.5/3/0.5), melting point range of 217~220° C.)) through hot-air blowing.

### Implementation on Mice

A coin battery (2 g, 140 mAh, Digi-Key electronics) is electrically attached to the flexible PCB (0.6 g) to power the device. MEA-hydrogel device (0.6 g) is electrically connected with PCB. A Python algorithm was written to wireless connected with the Bluetooth® modules and a user interface is created to command the device. After removing the magnet from the mice dorsal skin, the whole device was placed onto the skin, where the magnet has clipped. A 5 mA current command was given to the device for 6 min. As the placement of the battery and flexible PCB is heavy and may influence the movement of mice, we remove them after charging.

### Demonstration of Portability on Human

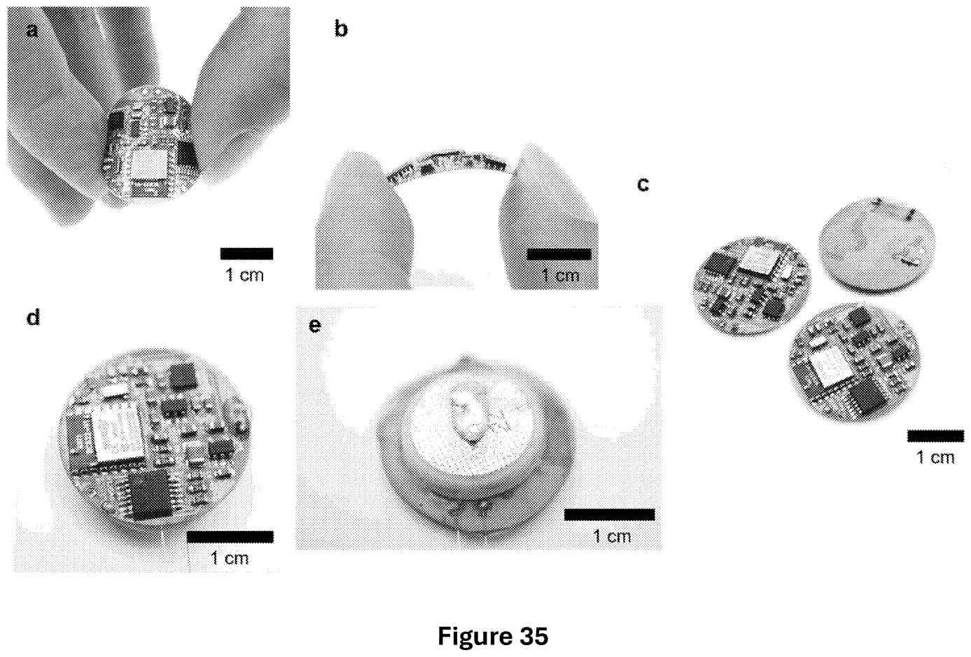

Coin battery is soldered onto the back of the flexible PCB (FIG. 35). MEA-hydrogel device (0.6 g) is electrically connected with the PCB. The whole device is placed into a 3D-printed ring. The final device assembly is placed onto arm to show its clinical translational potential on human pressure ulcer treatment (FIG. 2 (b)(vii) & (viii)).

### Example 1: H₂ Generation, Storage, and Diffusion in Hydrogel Electrochemical Cells

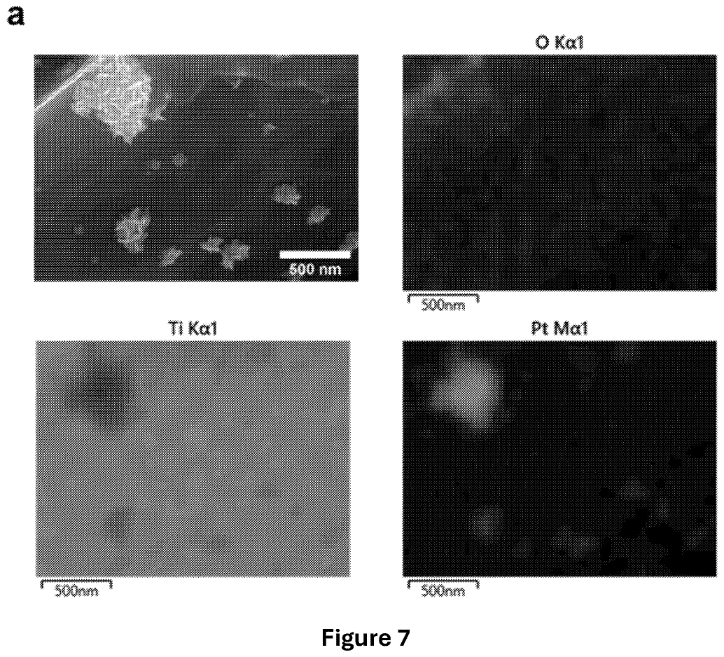

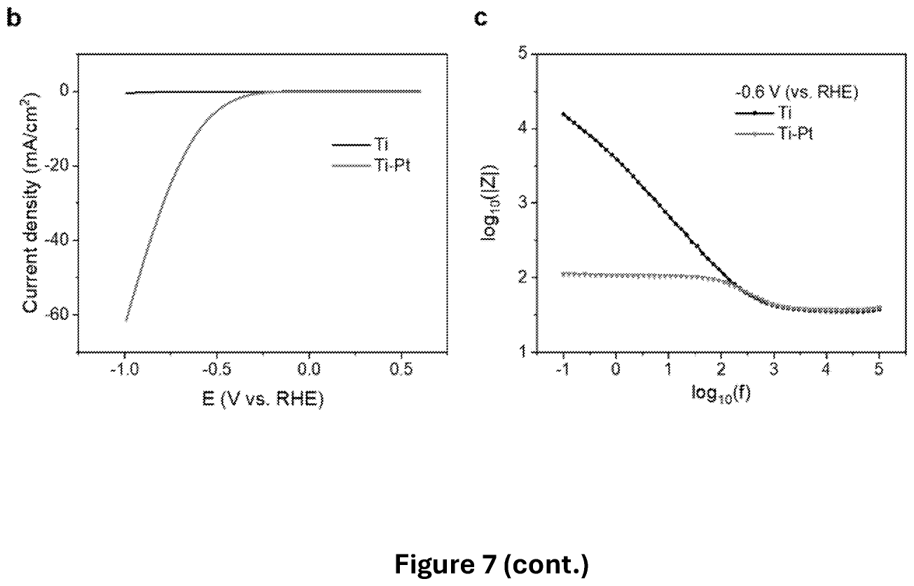

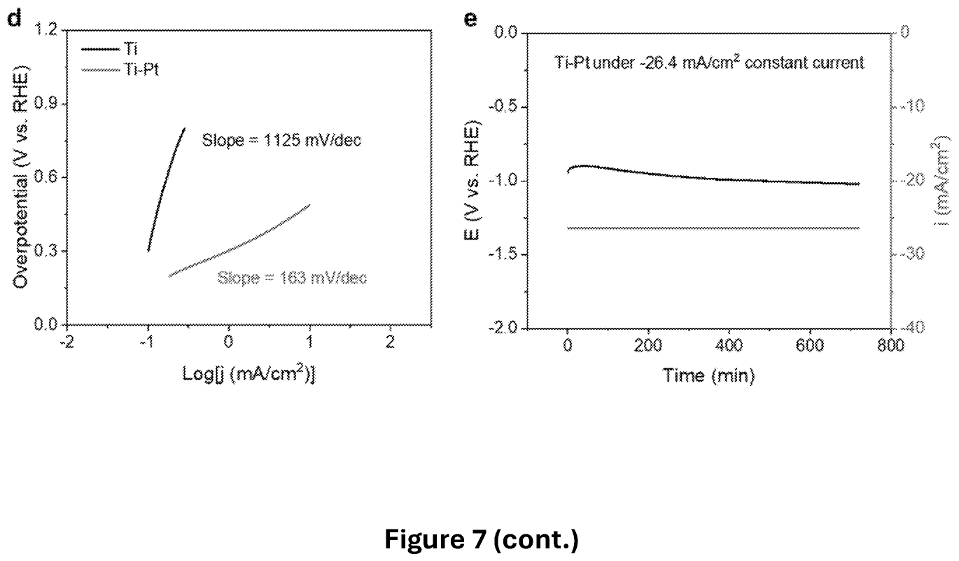

Platinum (Pt) was selected as the electrocatalyst for HER due to its superior catalytic efficiency and biocompatibility¹⁴⁻¹⁶. Scanning electron microscopy/energy dispersive X-ray spectroscopy (SEM/EDX) analysis confirmed the successful attachment of Pt nanoparticles on the titanium (Ti) electrode surface (FIG. 7 (a)). Electrochemical characterizations, including linear sweep voltammetry (LSV) and electrochemical impedance spectroscopy (EIS), showed higher current density and lower impedance for Ti—Pt electrodes compared to bare Ti (FIG. 7 (b), 1 (c)). The reduced Tafel slope of Ti—Pt electrodes indicated enhanced reaction kinetics and catalytic activity (FIG. 7 (d)). Chronopotentiometry (CP) tests showed stable reaction kinetics over 12 hours (FIG. 7 (e)).

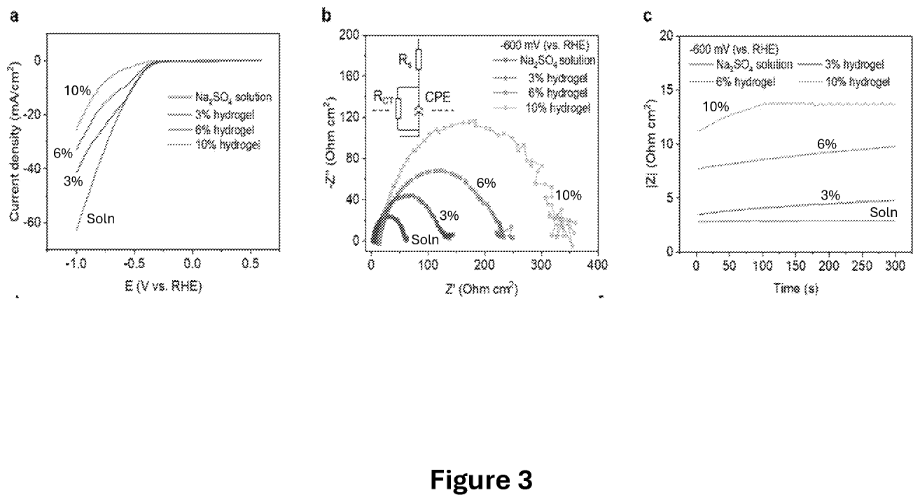

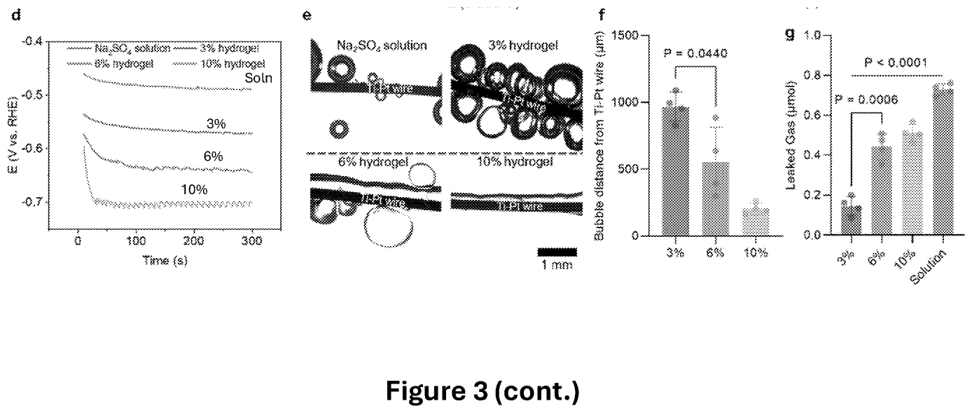

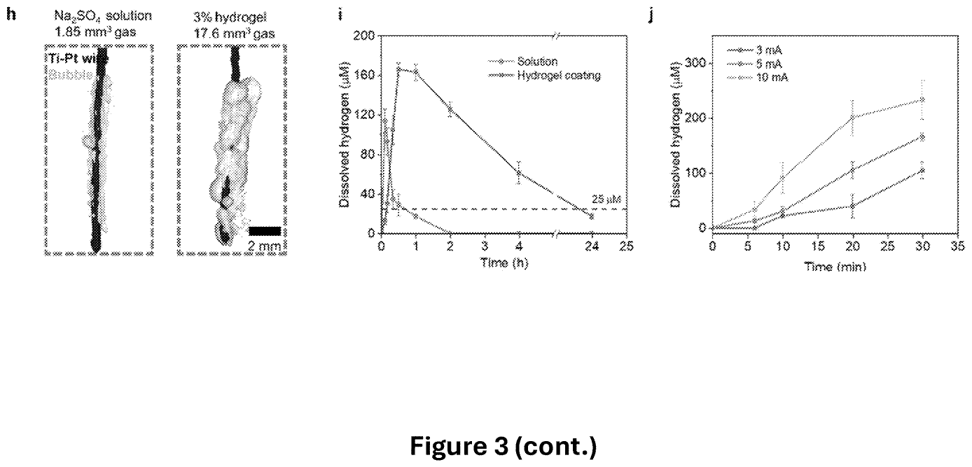

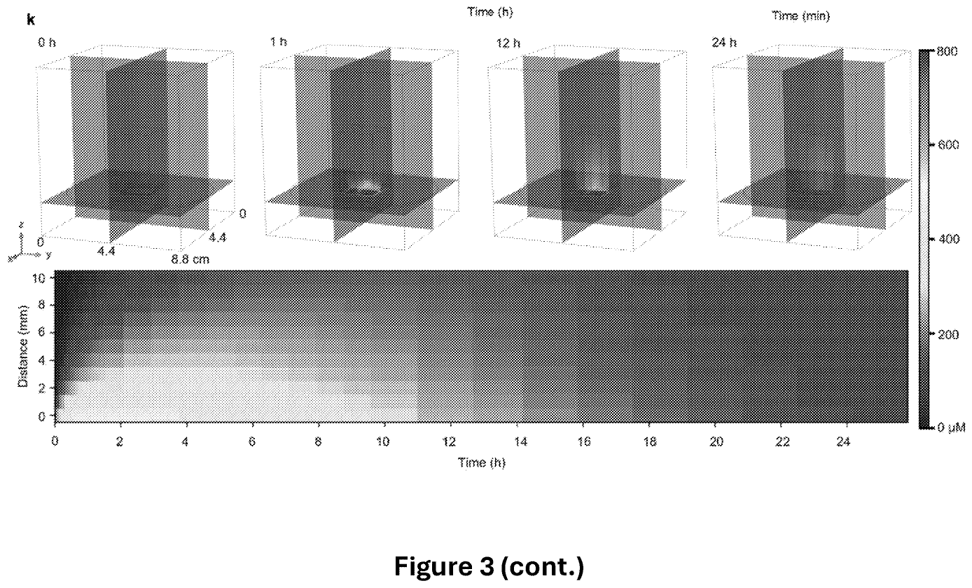

To assess electrode performance in hydrogel environments, polyvinyl alcohol (PVA) hydrogels with varying polymer concentrations (3%, 6%, and 10% PVA in 0.1 M Na₂SO₄ solution) were synthesized using a freeze-thaw method to physically crosslink the polymer. Electrically active chemical crosslinkers were intentionally avoided to prevent oxidation or reduction reactions caused by crosslinker residues. A key distinction between solution-based and hydrogel electrolytes is bubble dynamics during the HER. In 0.1 M Na₂SO₄ solutions without PVA, bubbles escape when buoyancy exceeds adhesion to the electrode¹⁷. In hydrogels, elastic forces trap bubbles either within the hydrogel matrix or at the electrode-hydrogel interface. Gas trapping and restricted electrolyte diffusion in the hydrogel network causes reduced current density, increased impedance, and dynamic instability (FIG. 3 (a-d)).

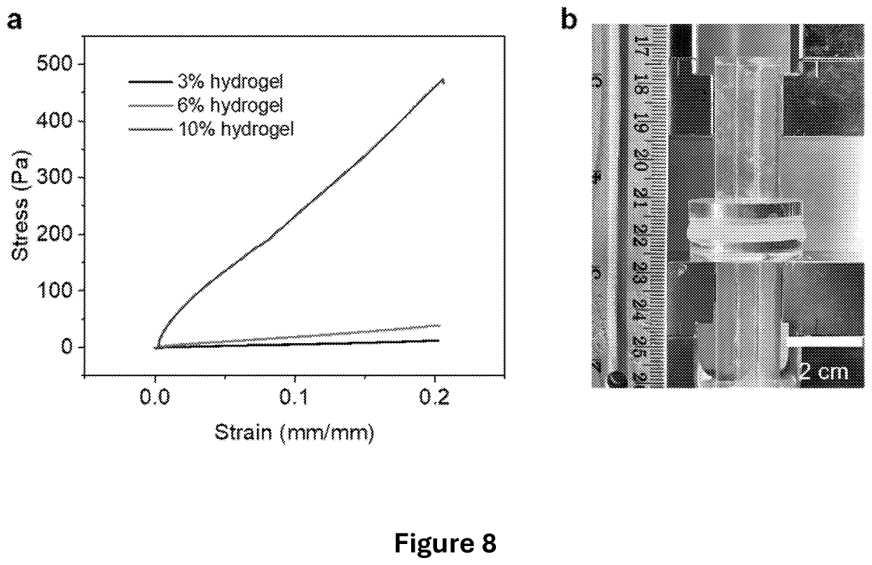

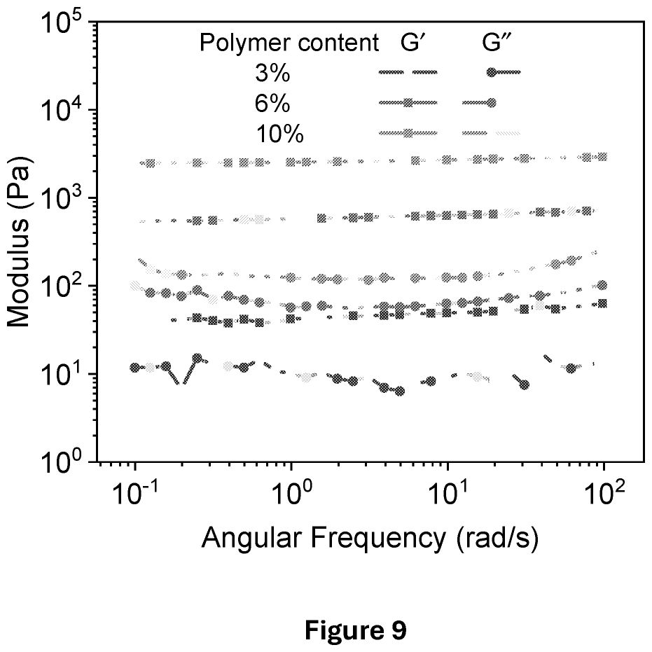

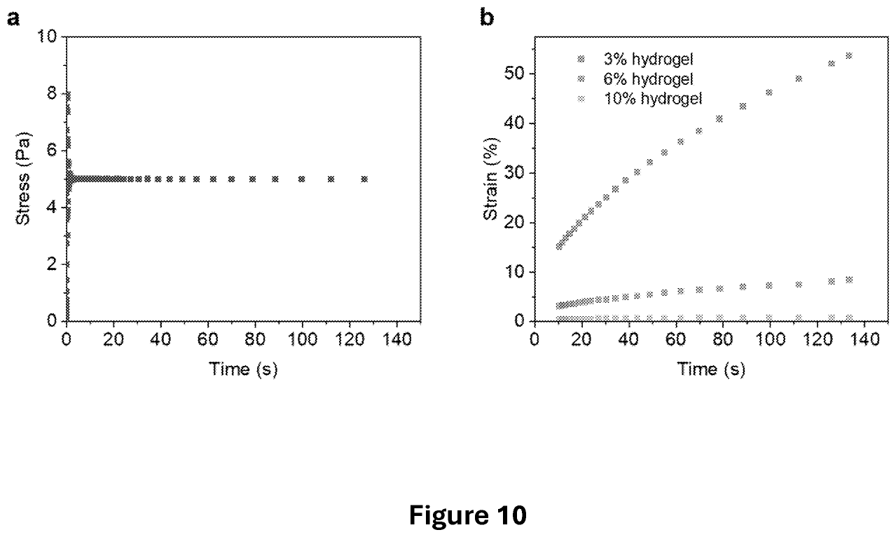

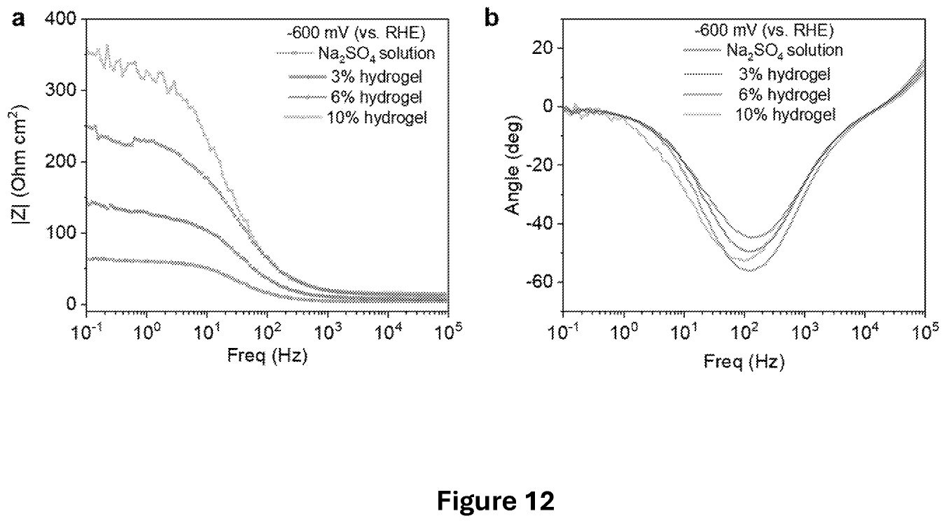

Bubble morphology depends on hydrogel polymer content and modulus. In 3% hydrogels (lowest modulus, FIGS. 8 and 9), bubbles remained discrete. Hydrogels with higher polymer content (i.e., higher modulus) exhibit smaller strain changes under the same applied stress (FIG. 10), compressing bubbles closer to the electrode surface and facilitating debonding of hydrogels from the electrode and H₂ gas channel formation (FIGS. 3 (e) and (f)), as confirmed by mechanical simulations (FIG. 11). Gas channel formation obstructs catalytic sites and reduces available electrode surface area, leading to further reduced current density and increased impedance as hydrogel polymer contents increases (FIGS. 3 (a) and (b), FIG. 12)^{18,19}.

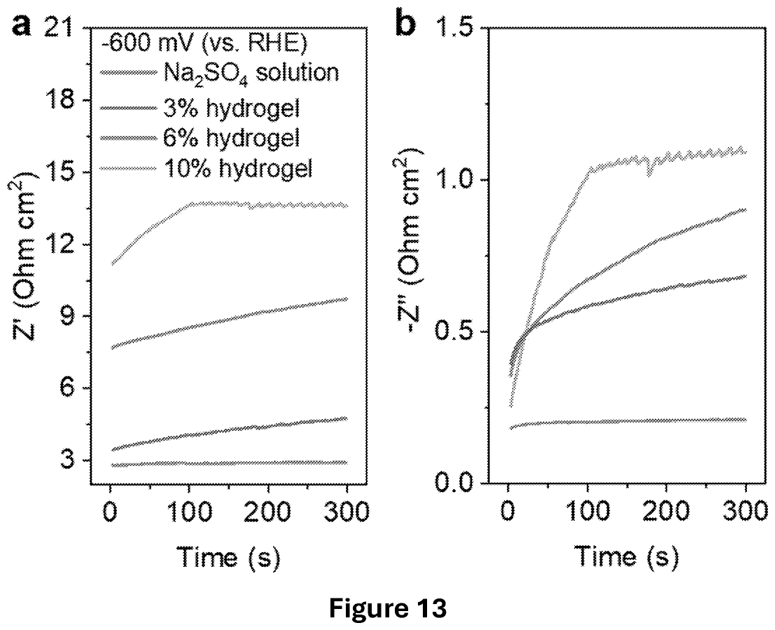

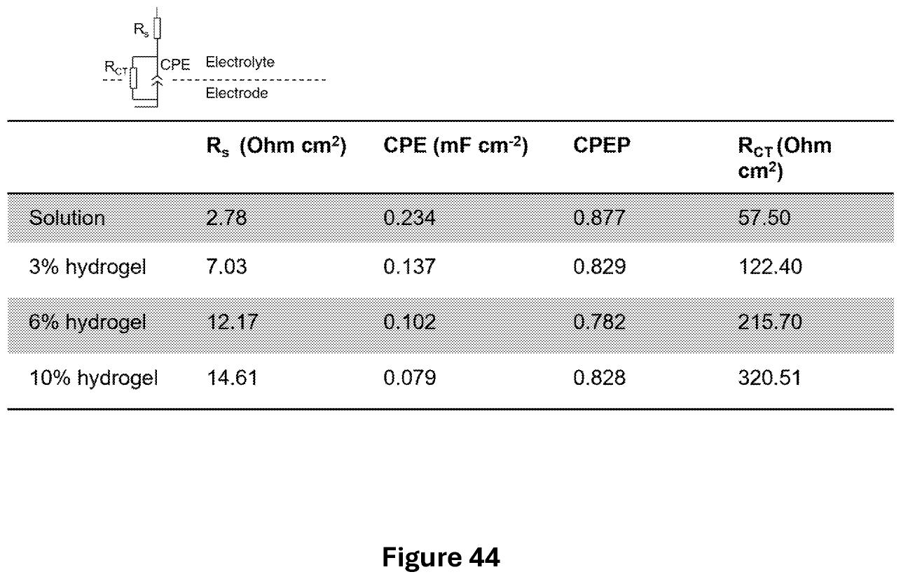

The EIS curves (from FIG. 3 (b)) were modeled using the Randles electrical equivalent circuit (EEC), with Rₛ, R_{CT} and CPE representing solution resistance, charge-transfer resistance, and constant phase element, respectively. The simulation results show increases in Rₛ and R_{CT} and a decrease in CPE as the polymer content of the hydrogel increases (FIG. 44), attributed to reduced electrode surface area and restricted electrolyte diffusion in denser polymer networks. The interfacial capacitance also decreases due to the lower dielectric constant of trapped gas (~1) compared to water (~78.2). Over time, bubble accumulation in hydrogel electrolytes leads to increasing real, imaginary, and total impedance, unlike stable behavior in solution electrolytes, as observed in electrochemical impedance time spectroscopy (EITS) (FIG. 3 (c), FIG. 13).

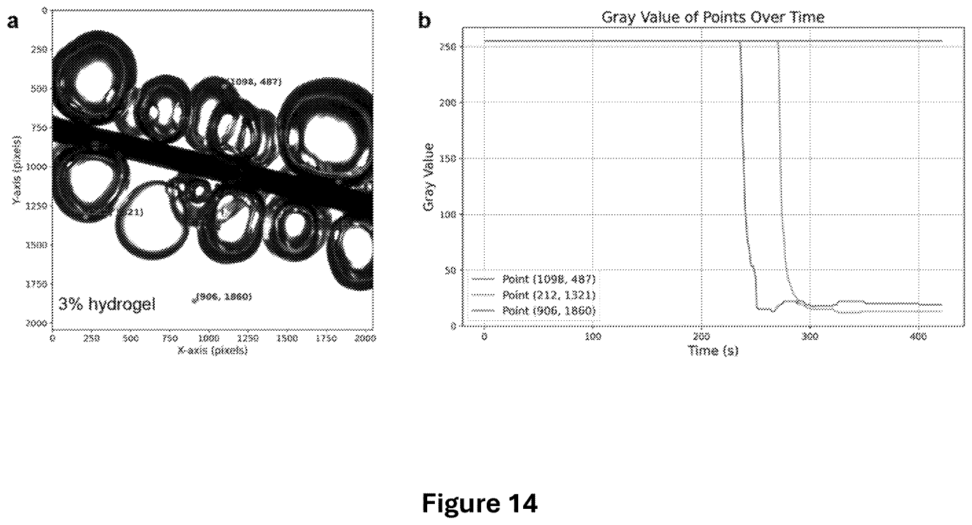

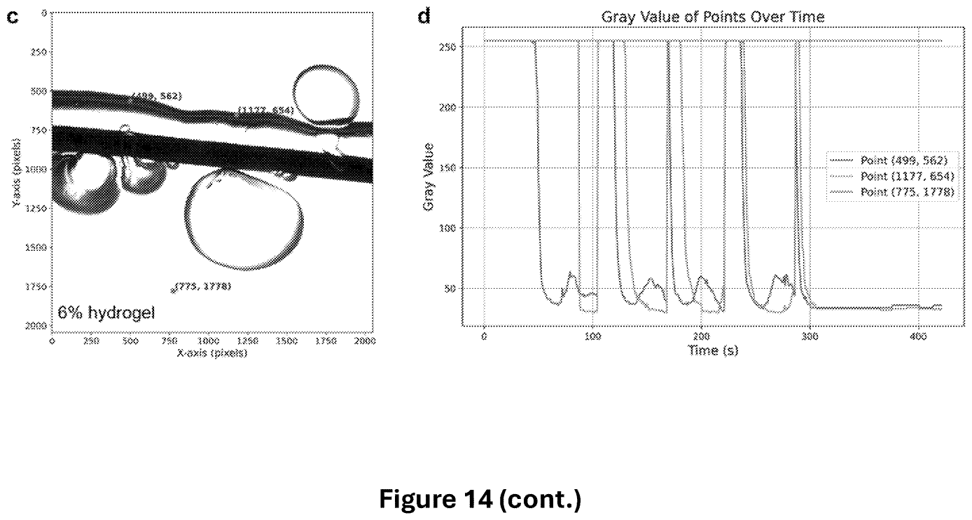

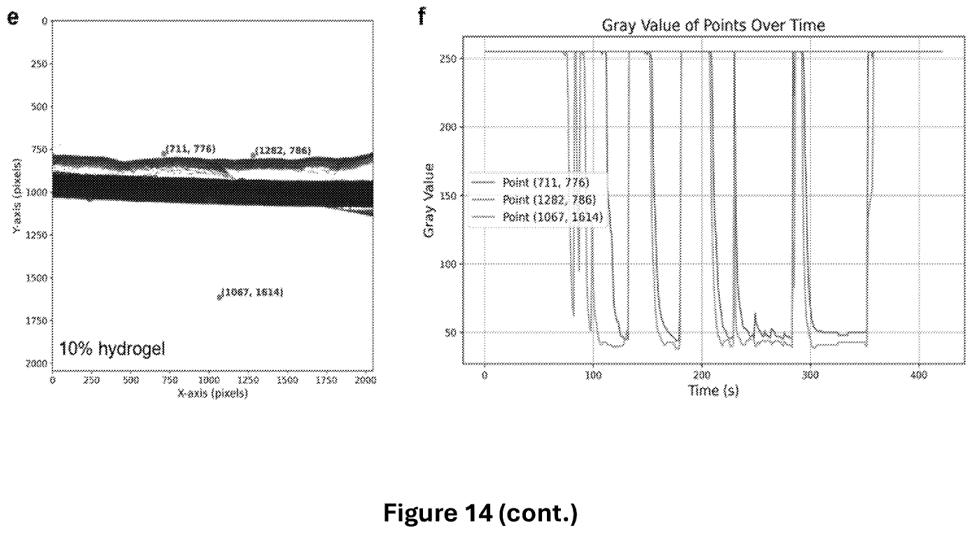

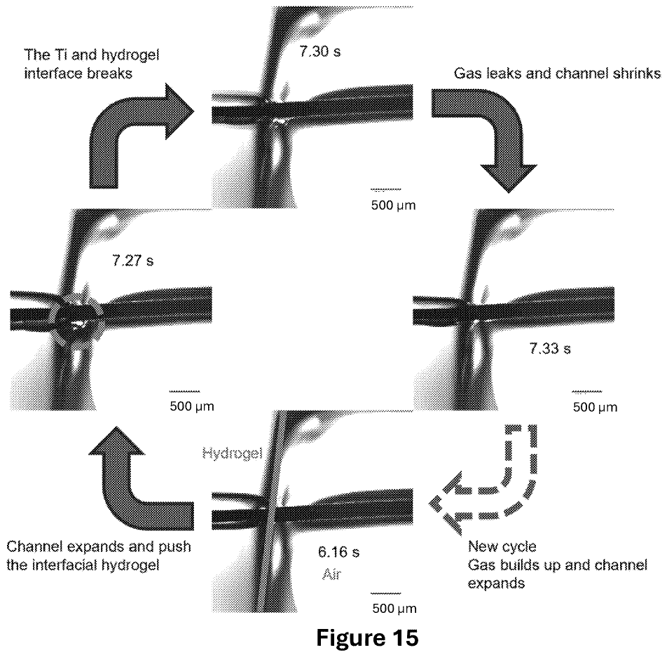

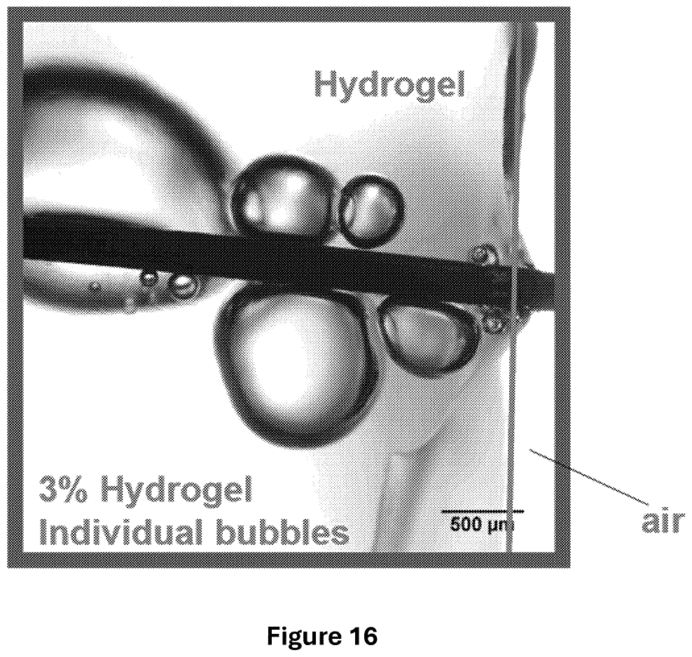

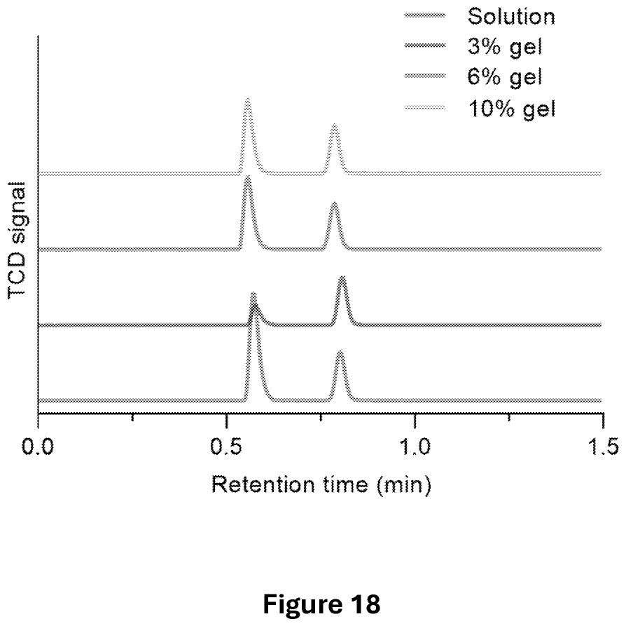

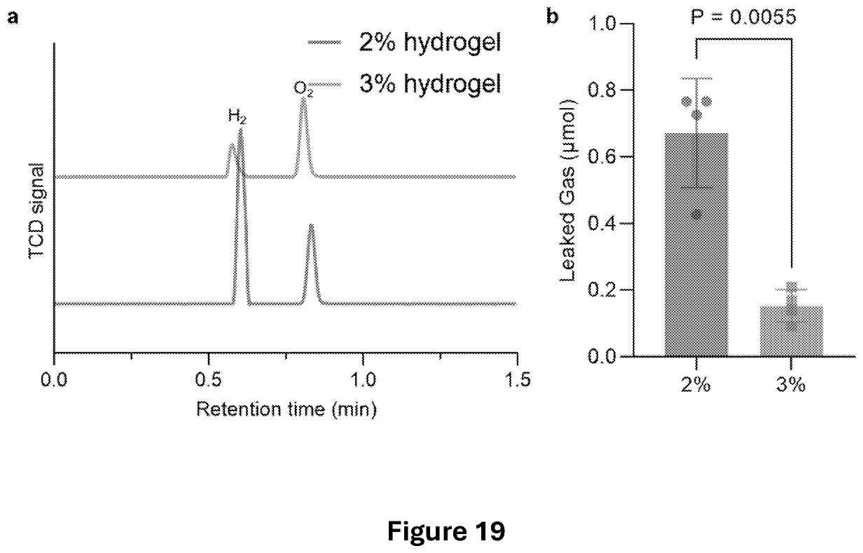

In the 6% and 10% hydrogel samples, voltage oscillations were observed when constant current was applied to the Ti—Pt electrode (FIG. 3 (d)). Similar impedance oscillations in EITS tests were also observed for the 10% hydrogel sample (FIG. 3 (c), FIG. 13). Video analysis revealed corresponding mechanical oscillations (FIG. 14), attributed to dynamic instability caused by quasi-periodic gas leakage from the gas channels at the Ti-hydrogel-air interface (FIG. 15). In contrast, the 3% hydrogel showed no voltage or mechanical oscillations, as gas bubbles remained distinct at the interface without forming interconnected gas channels (FIG. 16). This stability minimized gas leakage compared to the 6% and 10% hydrogels under identical charging conditions (FIGS. 3 (g), 17, and 18). Further reducing the polymer content to 2% resulted in liquid rather than gel following the freeze-thaw process, which was unable to trap gas effectively (FIG. 19). Based on its electrochemical performance and gas storage capability, the 3% hydrogel was therefore selected as the electrolyte for subsequent studies.

Current H₂ therapies rely on closed environments, confining cells or animals in closed chambers with specific H₂ and O₂ concentrations^{3,5,20,21}. This approach has limited clinical relevance due to the need for patient to be immobilized and the potential safety risks from H₂ flammability. Leveraging a hydrogel-based gas trapping mechanism, we developed an open-system electrochemical device for sustained hydrogen delivery. Micro-computed tomography (micro-CT) analysis of a Ti—Pt wire immersed in 3% hydrogel or Na₂SO₄ solution electrolyte under −0.5 mA for 5 minutes revealed that 17.6 mm³ of gas was trapped in the hydrogel, compared to minimal retention in the solution (FIG. 3 (h)), closely matching the theoretical yield of 18.7 mm³ at room temperature and pressure (RTP).

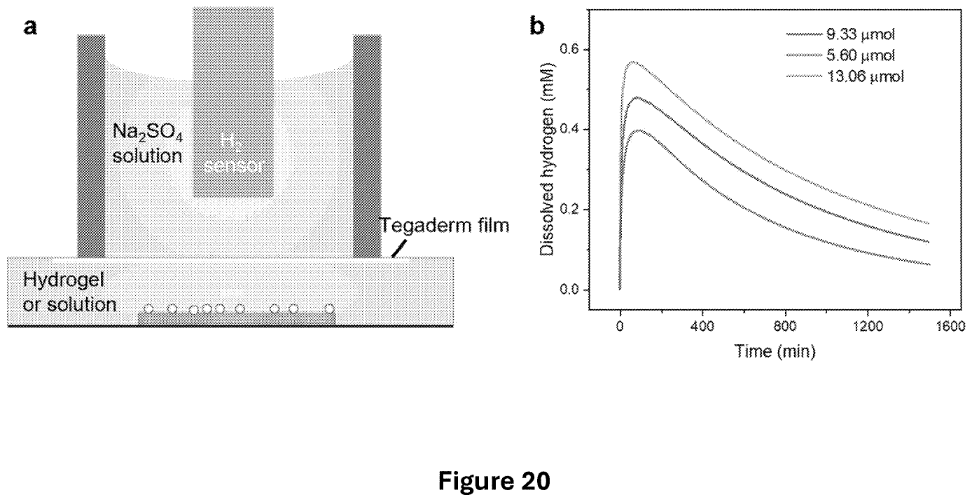

The gas retention properties of the hydrogel result in a distinct H₂ diffusion profile. To gain more insight into hydrogel-enabled diffusion, we designed a H₂-sensing setup utilizing a Ti mesh electrode to enhance H₂ production and a 3M Tegaderm™-sealed chamber to separate the electrochemical cell from a sensor-containing solution (FIG. 20 (a)). The Ti mesh-hydrogel electrochemical cell was engineered by electrodepositing Pt nanoparticles onto the Ti mesh and coating it with a 1.3 mm-thick 3% PVA hydrogel. Measurements of H₂ diffusion through the Tegaderm™ film revealed a higher H₂ concentration peak and significantly prolonged release with the hydrogel coating, compared to Na₂SO₄ solution, extending the reported effective treatment ([H₂]>25 μM)³ duration nearly 36-fold, from 0.65 hours to 23.5 hours, with a small energy input of 0.5 mAh (FIG. 3i). The sustained diffusion and therapeutic concentration of H₂ enabled by the hydrogel indicates the potential for greater H₂ utilization efficiency. The delivery profile may be further tuned by adjusting the applied current, demonstrating the versatility of the hydrogel for clinical H₂ therapies (FIG. 3 (j)). Diffusion simulations corroborated these findings, showing peak H₂ concentrations at approx. 1 hour, followed by a gradual decline over 24 hours (FIG. 3 (k), FIG. 20 (b)). Notably, residual dissolved H₂ persisted in the hydrogel after 24 hours, highlighting its potential utility in prolonged therapeutic applications.

### Example 2: H₂ Delivery Via Hydrogel Electrochemical Cell Mitigates Oxidative Damage

Hydroxyl radicals (•OH) are a highly reactive and damaging ROS with strong affinity for electron-rich biomolecules, including proteins and DNA²². To test the efficiency of the Ti-mesh hydrogel electrochemical cell in reducing •OH, we utilized the Fenton reaction (Fe²⁺+H₂O₂═Fe³⁺+•OH+OH⁻) in a cell-free system wherein •OH is generated upon introduction of H₂O₂ into an Fe(ClO₄)₂ solution at a specific time point³. Hydroxyl phenyl fluorescein (HPF) was used as a fluorescent probe to monitor changes in •OH concentration. As shown in FIGS. 3a and 3b, the addition of H₂O₂ to the Fe(ClO₄)₂ solution caused a rapid increase in •OH levels, while the addition of an equivalent volume of PBS had no effect. Pre-treatment of the Fe(ClO₄)₂ solution with H₂, delivered using the Ti mesh-hydrogel electrochemical cell, significantly reduced •OH levels, demonstrating the effectiveness of H₂ in neutralizing hydroxyl radicals.

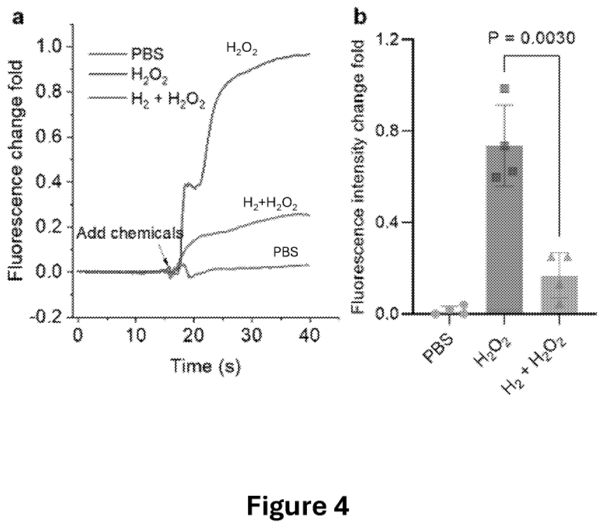

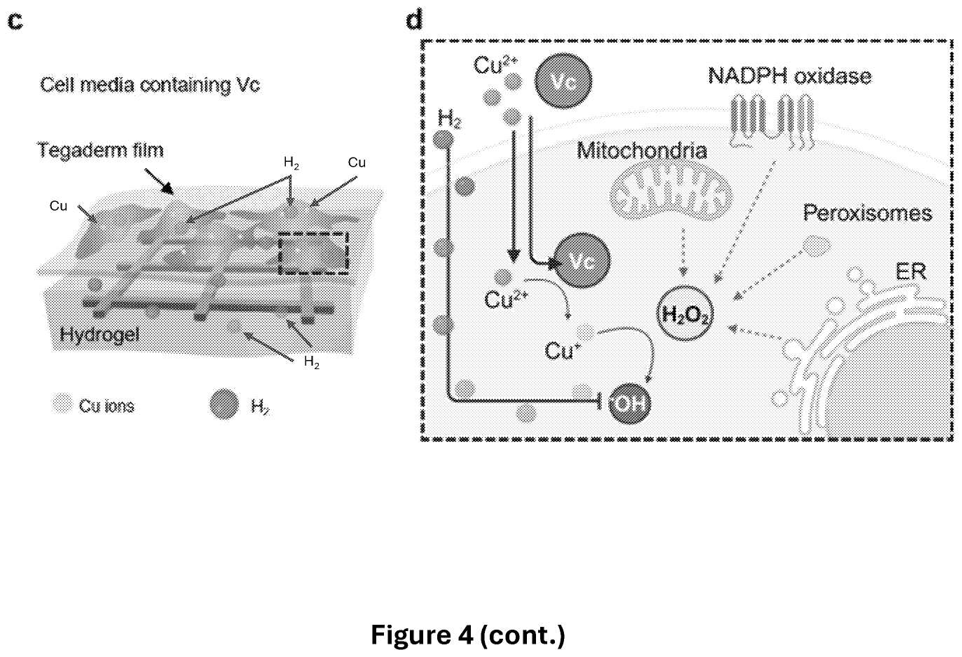

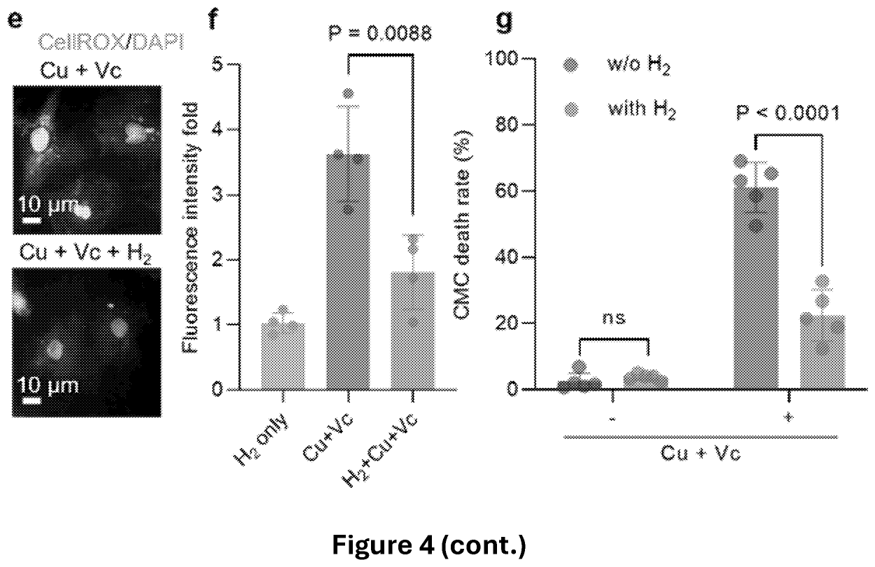

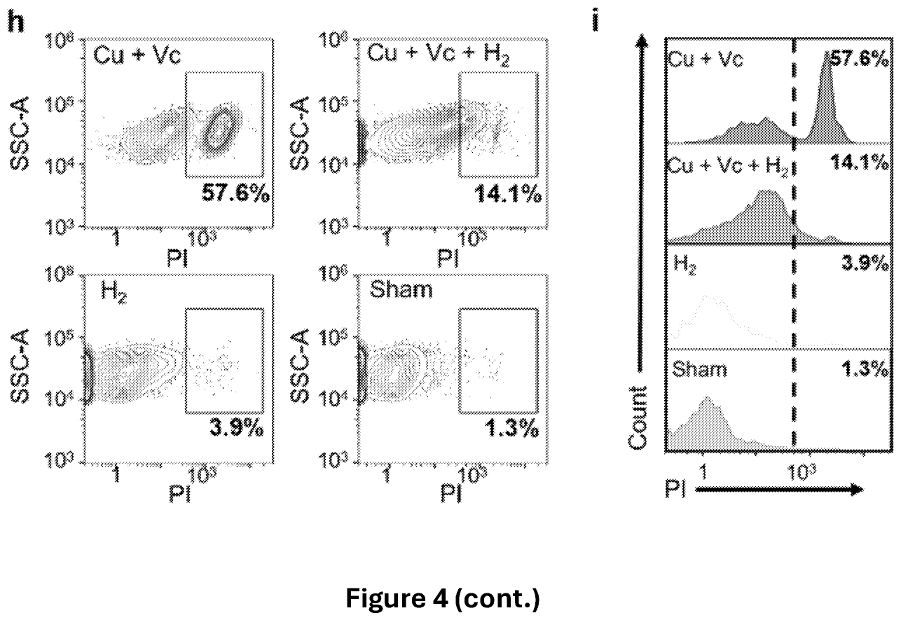

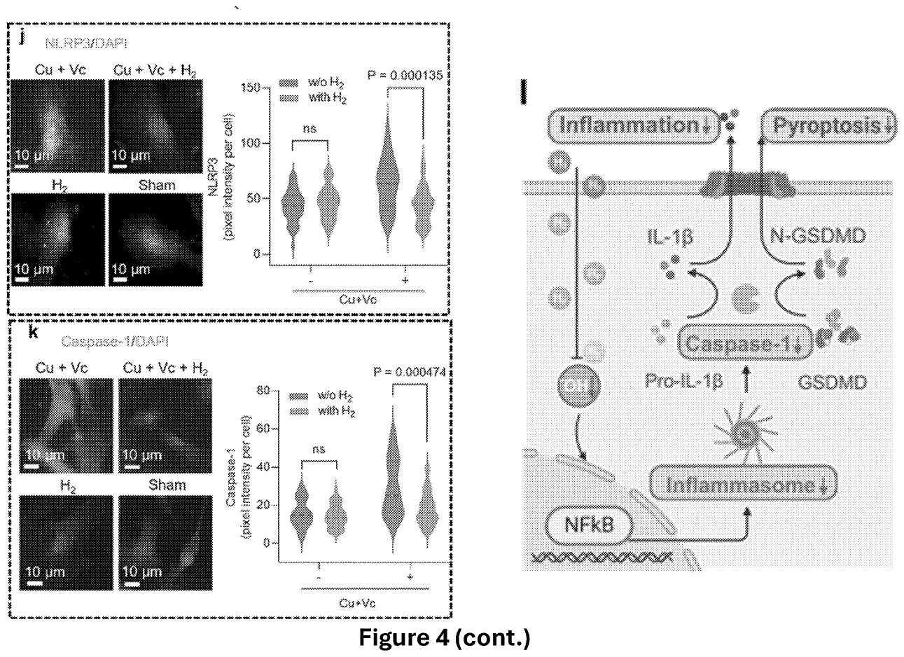

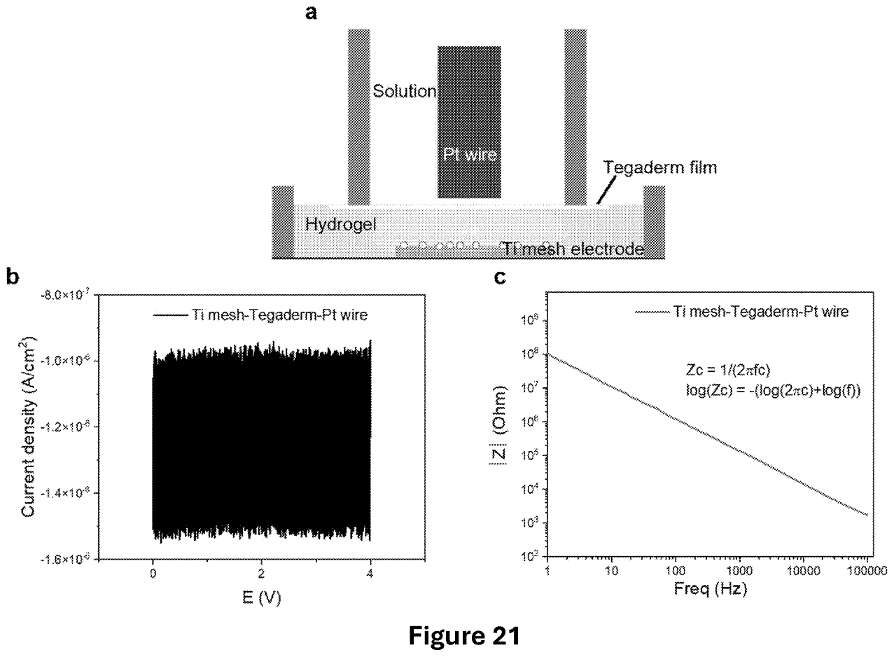

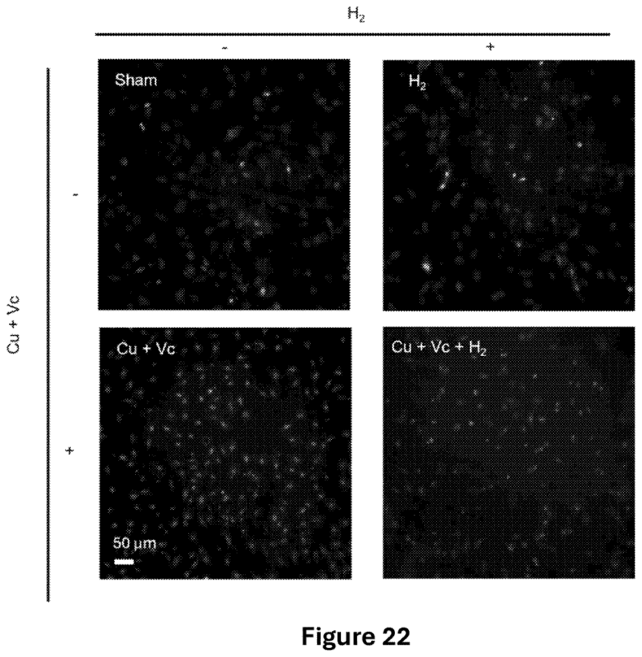

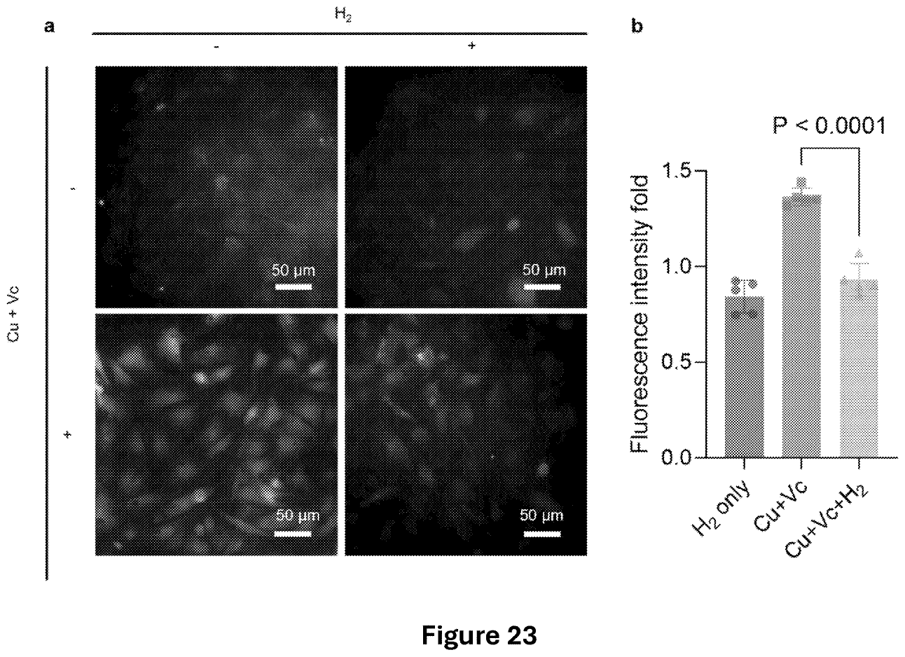

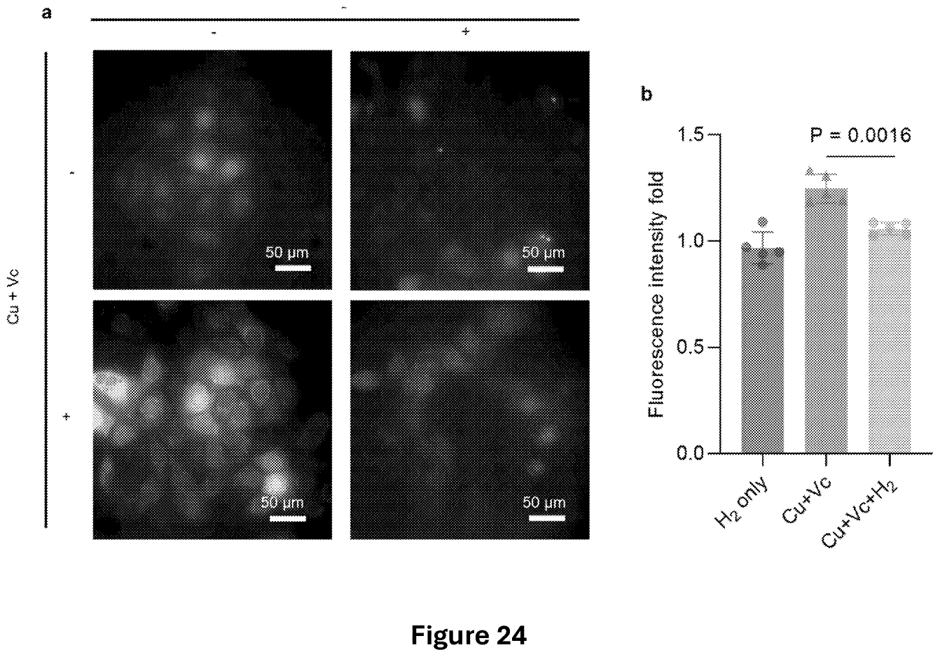

To verify the H₂ effect in vitro prior to organ-level application, we developed a cell model using a copper-based Fenton reaction to convert endogenous H₂O₂ into •OH, inducing severe oxidative stress (FIGS. 4 (c) and (d))³. Cardiomyocytes (CMs) or HaCaT keratinocytes were seeded on a Tegaderm™ membrane, which permits H₂ diffusion but prevents electrolyte exchange and electricity leakage (FIG. 21). The Ti mesh-hydrogel setup generated, stored, and released H₂ to cells (FIG. 4 (c)). Intracellular ROS levels, measured by CellROX™ and H₂DCFDA probes, were significantly elevated in cells treated with copper ions and vitamin C (Cu+Vc) relative to controls but were markedly reduced with H₂ exposure (FIGS. 4 (e) and (f), and 22-24).

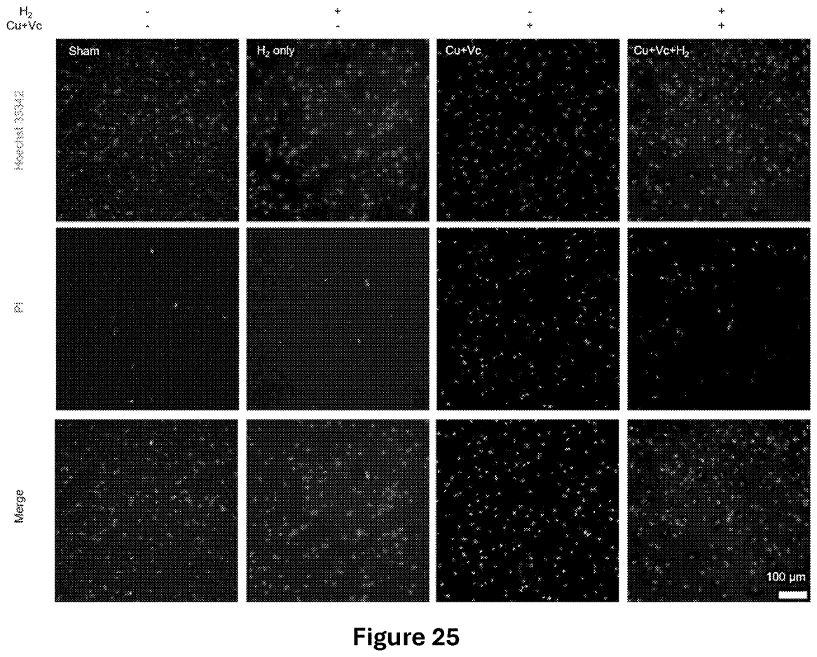

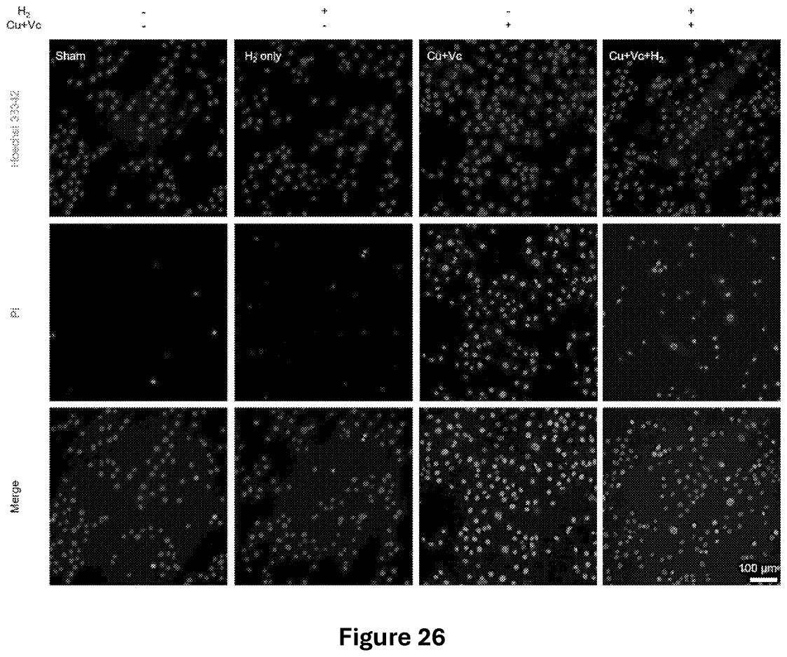

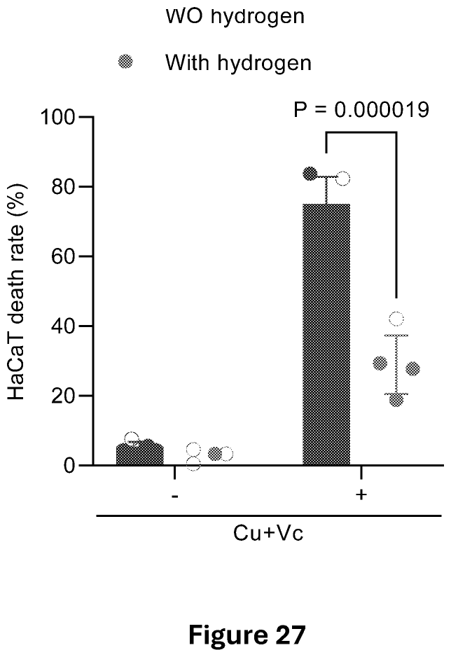

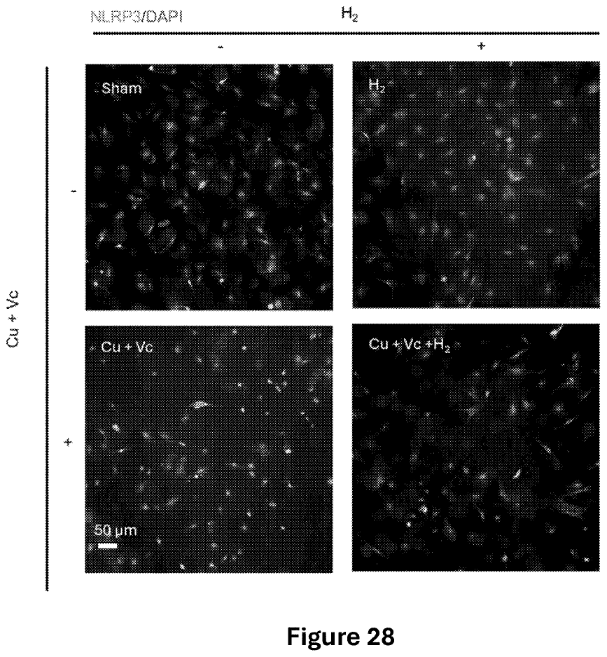

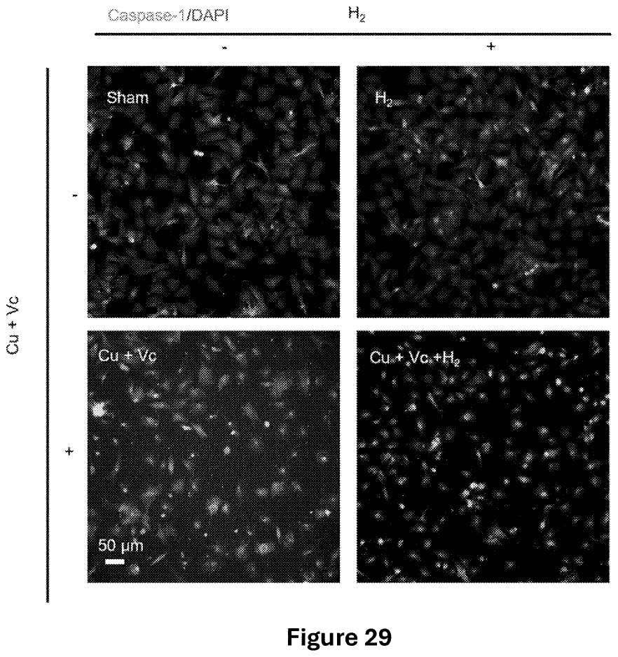

We evaluated cell viability using propidium iodide (PI)/Hoechst staining and PI flow cytometry in both CMs and HaCaT cells under Cu+Vc-induced oxidative stress. H₂ treatment reduced CM death from ~60% to ~25% (FIG. 4g, FIG. 25) and HaCaT cell death from ~70% to ~30% (FIGS. 26 and 27). Flow cytometry confirmed these findings in CMs (FIGS. 4 (h) and (i)).

Expression levels of inflammatory markers NLRP3 and Caspase-1 were markedly decreased in H₂-treated CMs subjected to Cu+Vc-induced oxidative stress relative to untreated controls (FIGS. 4(j) and (k), 28, and 29). No significant difference emerged between sham and H₂-only groups. These findings suggest that H₂, delivered by a Ti mesh-hydrogel bioelectronic system, modulates the NF-κB signaling pathway, thereby reducing inflammation, protecting cells from oxidative damage, and improving cell survival (FIG. 4 (l))²³.

### Example 3: H₂ Delivery Via MEA-Hydrogel Device Mitigates Ex Vivo Heart I/R Injury

To explore the therapeutic potential of our hydrogel bioelectronic device at the tissue level, we used the clinically relevant myocardial I/R injury model characterized by excessive ROS generation and exacerbated tissue damage^{24,25}.

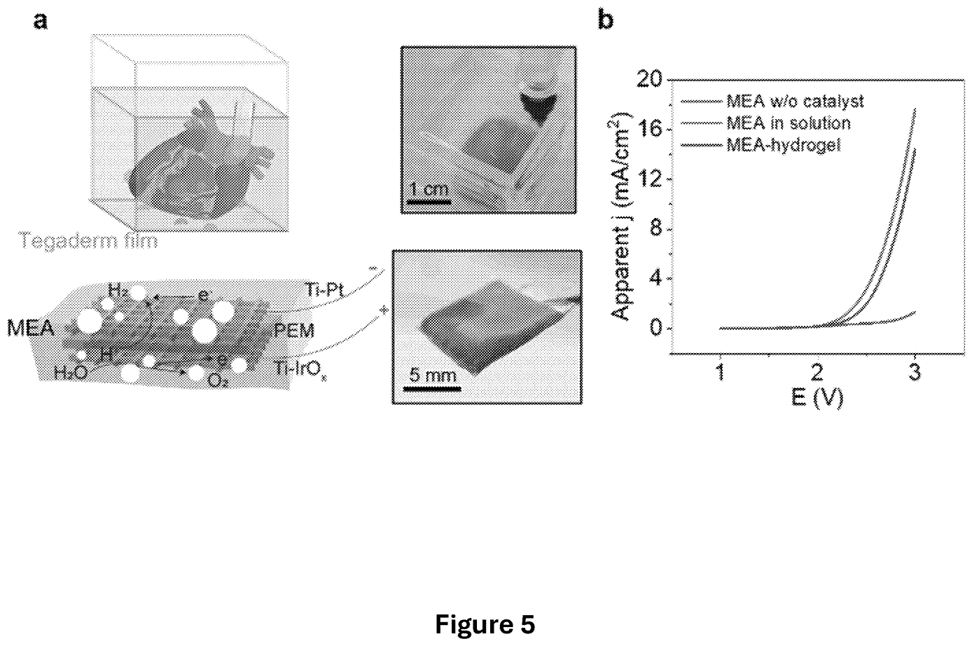

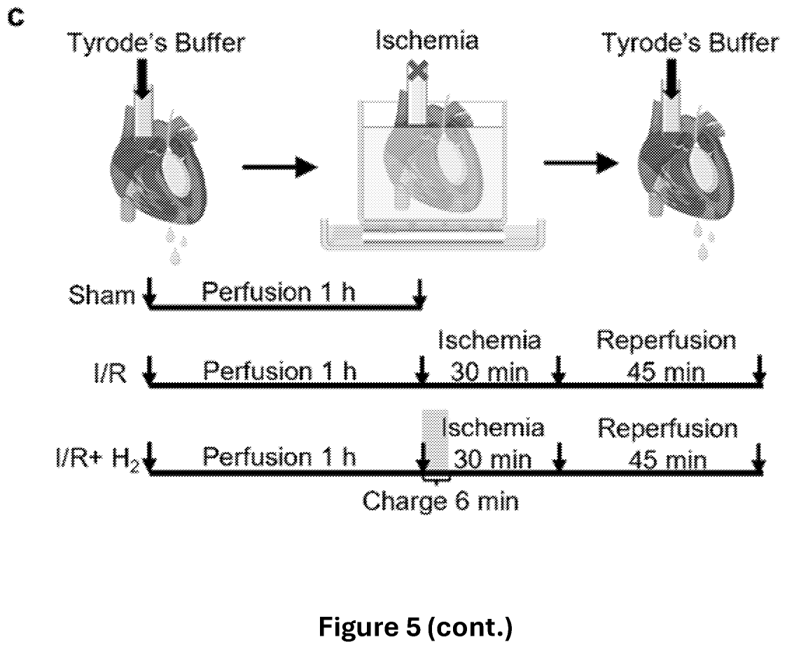

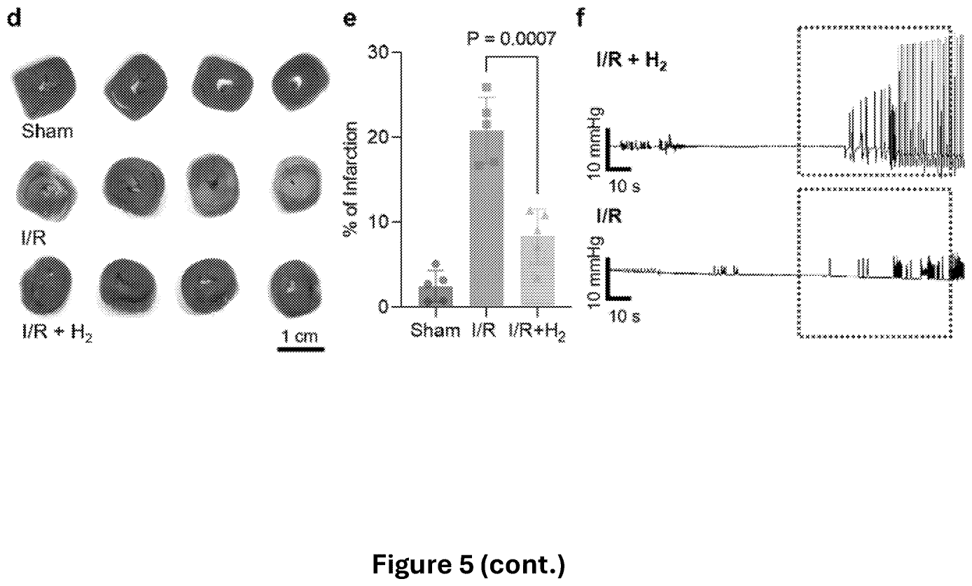

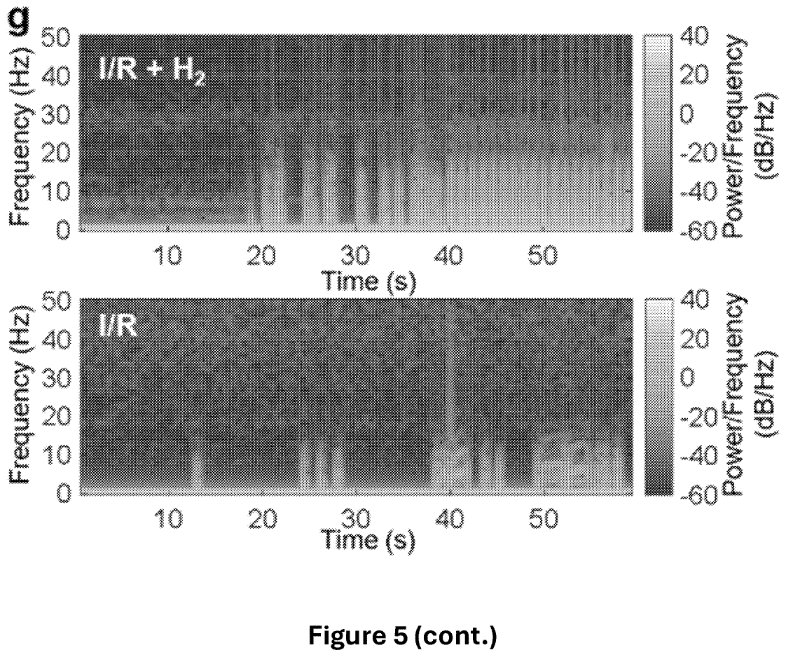

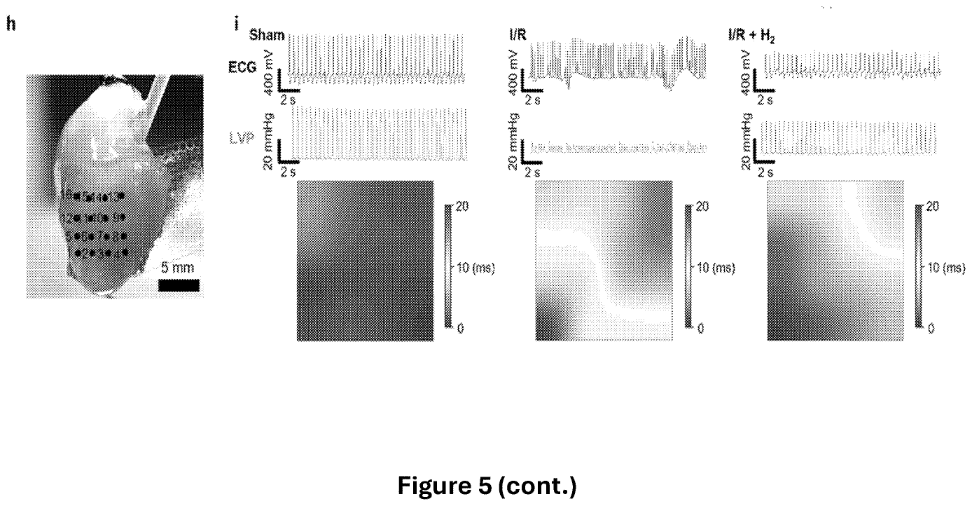

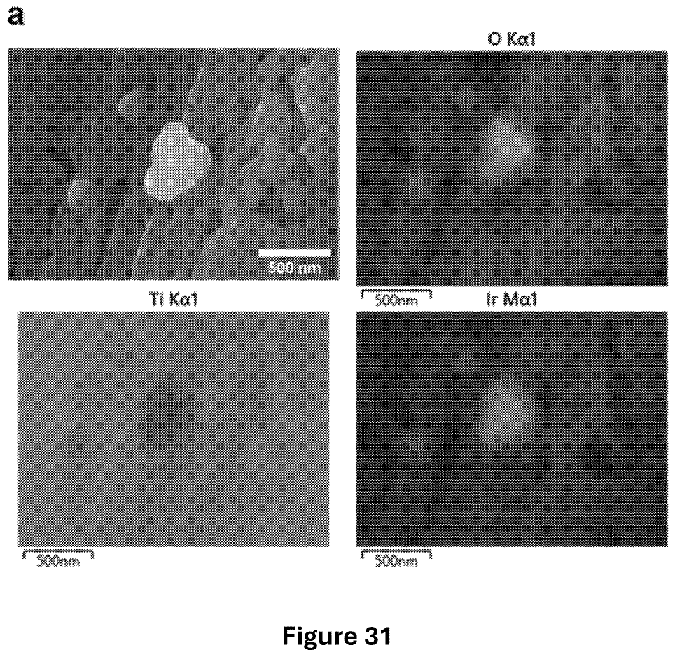

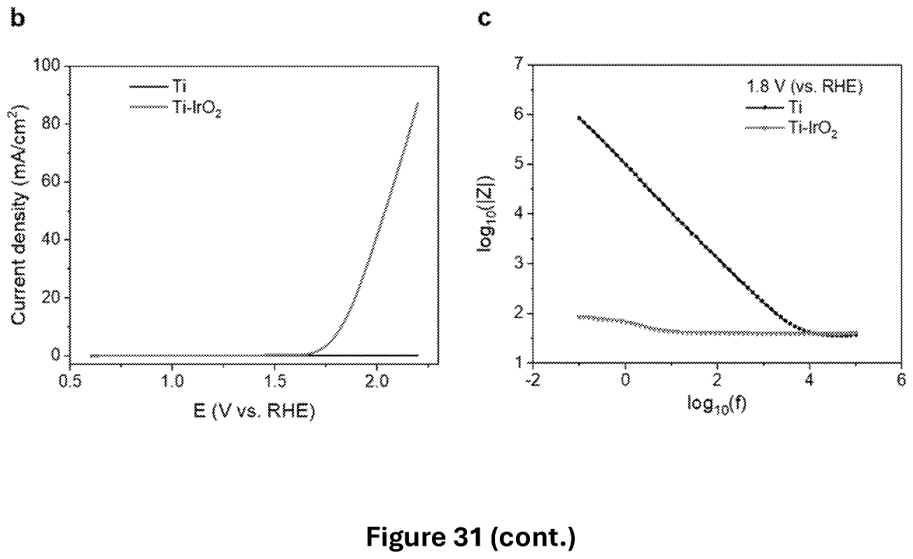

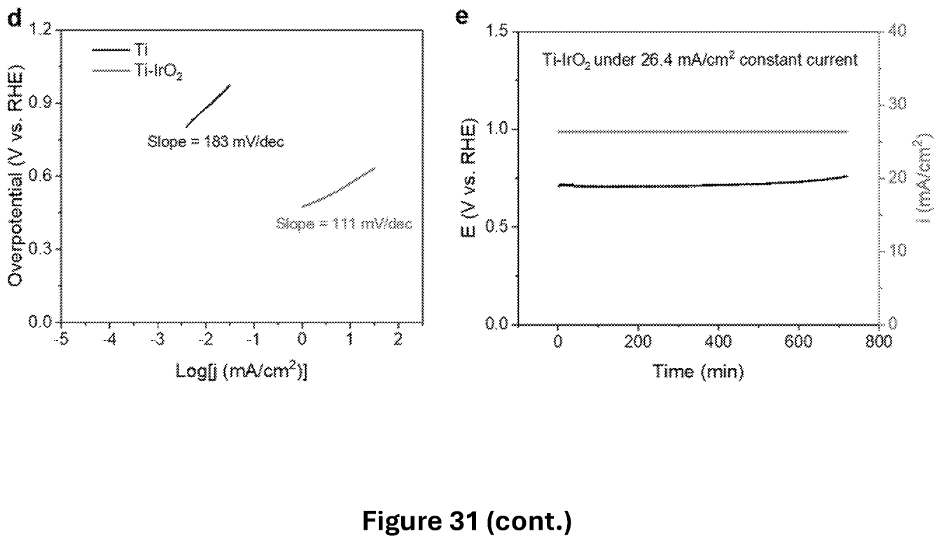

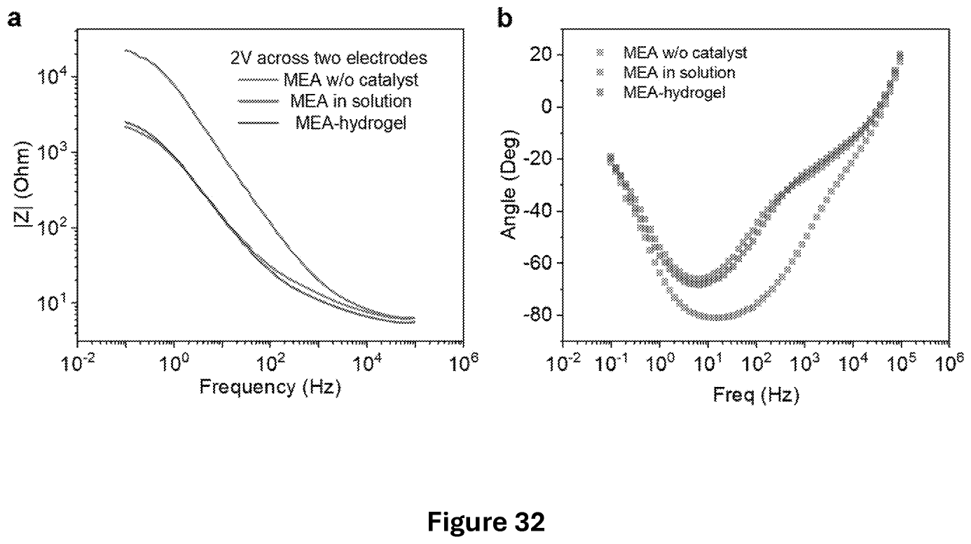

We designed a membrane electrode assembly-hydrogel (MEA-hydrogel) configuration as a self-contained hydrogel bioelectronic device without external reference and counter electrodes (FIG. 5 (a), lower panel). This compact system incorporates Ti—Pt and Ti—IrO₂ mesh electrodes on opposite faces of a Nafion™ PEM, all embedded in a hydrogel matrix, resulting in a portable bioelectronic device for water splitting (FIG. 30). We thoroughly evaluated and confirmed the stability and oxygen evolution reaction (OER) efficiency of the IrO₂ catalyst (FIG. 31). Proton conduction through the PEM drives H₂ generation on the cathodic Ti—Pt side. Electrodeposition of Pt and IrO₂ on the respective electrodes improved electrochemical performance and reduced impedance (FIG. 5 (b), FIG. 32). While the hydrogel coating slightly reduces MEA device performance, it provides beneficial gas trapping and diffusion properties essential for therapeutic H₂ delivery.

We assessed the therapeutic impact of the MEA-hydrogel device using a Langendorff I/R model (FIGS. 5 (a) and (c))²⁶. A saline-filled balloon was inserted into the left ventricle of the isolated Langendorff heart to enable real-time pressure monitoring of left ventricular pressure (LVP). Hearts were perfused with Tyrode's buffer for one hour. Ischemic injury was induced by halting the perfusion flow for 30 minutes, followed by reperfusion for 45 minutes. During ischemia, the isolated heart was placed onto Tegaderm™ chamber, with the MEA-hydrogel device underneath. A brief 6-minute, 5 mA charge generated H₂ within the hydrogel, enabling sustained release into the cardiac tissue. We performed a H₂ diffusion simulation with a 3D-scanned rat heart model inside Tegaderm™ chamber. The result revealed that the region of heart in contact with the Tegaderm™ film received a great amount of H₂ during the 30-min ischemia (FIG. 33), which could eliminate the localized •OH ROS that surges post reperfusion^{27,28}. After I/R injury, heart slices were stained with triphenyltetrazolium chloride (TTC) to detect infarcted areas. Hearts treated with H₂ exhibited a significant reduction in infarct size compared to the control group without H₂ treatment (FIGS. 5 (d) and (e)).

Functional assessments confirmed these histological findings. LVP measurements and spectrogram analysis showed that H₂-treated hearts recovered regular contractile activity with robust contraction pressure and stable contraction period within 3 min after the start of reperfusion (FIGS. 5f, 5g). After 45 minutes of reperfusion, H₂-treated hearts exhibited heart rates, LVP, and electrocardiogram (ECG) patterns similar to healthy, pre-injury controls. In contrast, untreated I/R hearts displayed arrhythmias and diminished contractility (FIG. 5i).

To directly assess electrical conduction, a flexible 16-channel microelectrode array recorded epicardial potentials (FIG. 5 (h)). Isochrone maps after reperfusion demonstrated markedly delayed activation in I/R hearts, whereas H₂-treated hearts showed conduction patterns with smaller delay time (FIG. 5 (i)).

Collectively, these results confirm that H₂ generated via our MEA-hydrogel bioelectronic device substantially protects against I/R injury. This treatment reduces infarct size, enhances cardiac contractile recovery, and restores electrical conduction. Such advances highlight the device's potential as a clinically relevant, bioelectronic solution for managing oxidative stress-induced cardiac damage, beneficial for patients at high risk of heart tissue damage, such as severe coronary artery disease or undergoing major heart surgeries²⁹.

### Example 4: H₂ Delivery Via Portable H-Pad for In Vivo Treatment of I/R Skin Pressure Ulcers

Pressure ulcers (bedsores) present a global healthcare challenge, causing severe patient discomfort and reduced quality of life. In the United States, approximately 60,000 annual fatalities are attributed to pressure ulcers, exceeding suicide-related deaths, and incurring an estimated $22 billion in healthcare costs as of 2014³⁰⁻³². Despite these metrics, pressure injuries have received relatively limited public health attention. Advancements in novel dressings and therapies have shown limited additional benefits, and the treatment remains vague and time-consuming, underscoring an urgent need for improved preventive and therapeutic strategies³³. Here, we introduce a hydrogel bioelectronic device designed to mitigate pressure injuries and enhance clinical outcomes.

The pathophysiology of pressure ulcers involves mechanical loading (e.g., pressure, shear, friction) and prolonged immobility, resulting in restricted blood flow and localized ischemia³⁴. Ischemia impairs mitochondrial electron transport, leading to excessive ROS generation. When the patient's body position changes, and blood flow is restored, reperfusion further increases ROS production³⁵. This surge in ROS overwhelms the tissue's antioxidant defenses, initiating oxidative stress, inflammation, disrupting cytokine signaling, delaying wound healing, and ultimately leading to tissue necrosis.

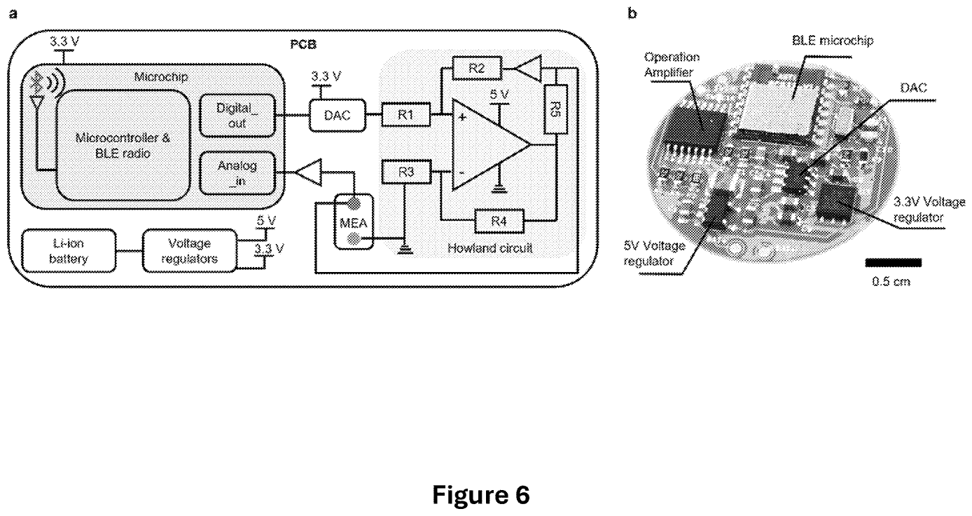

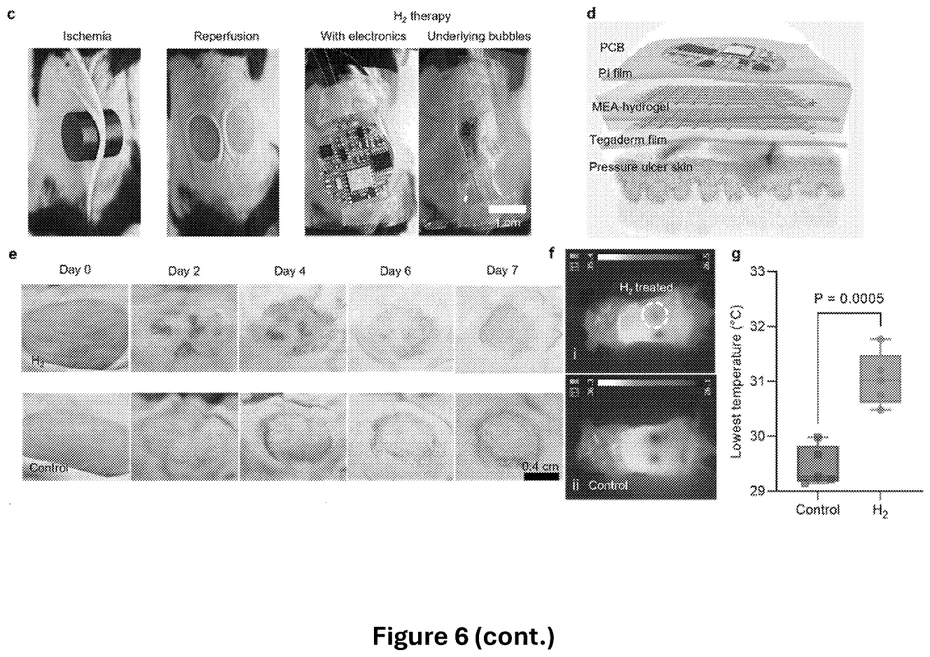

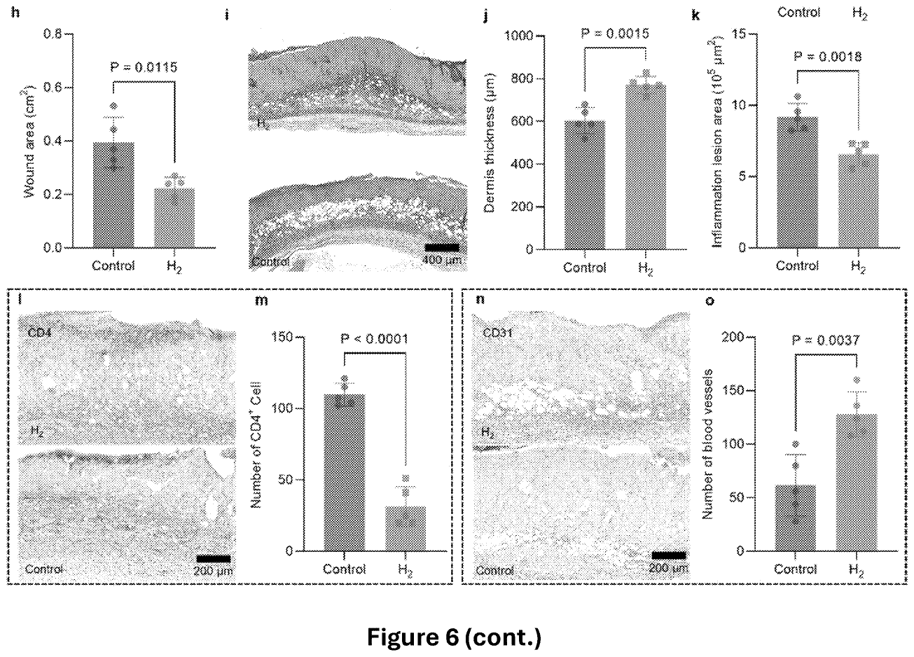

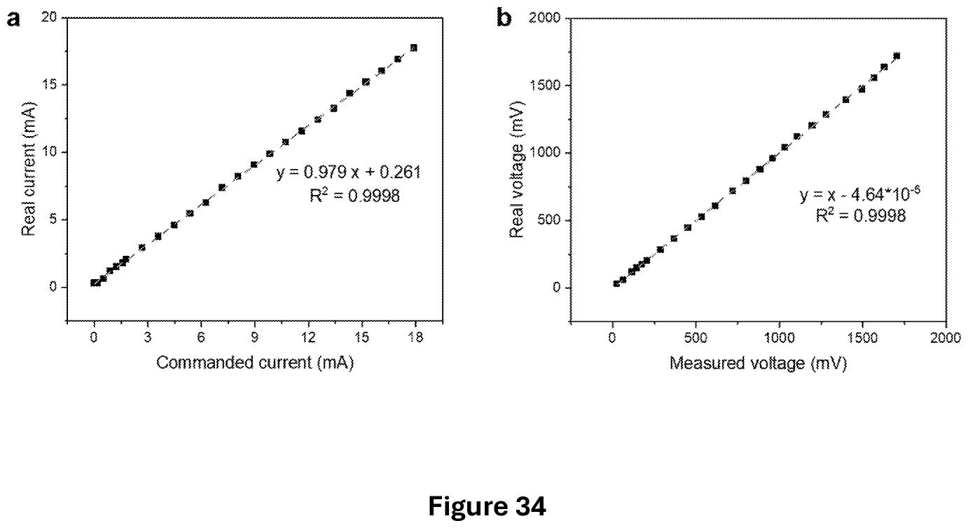

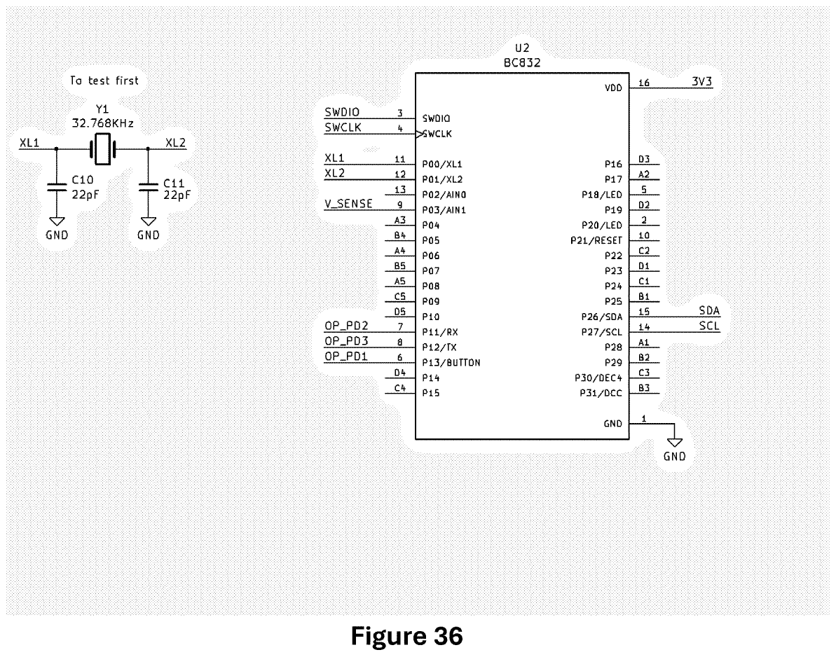

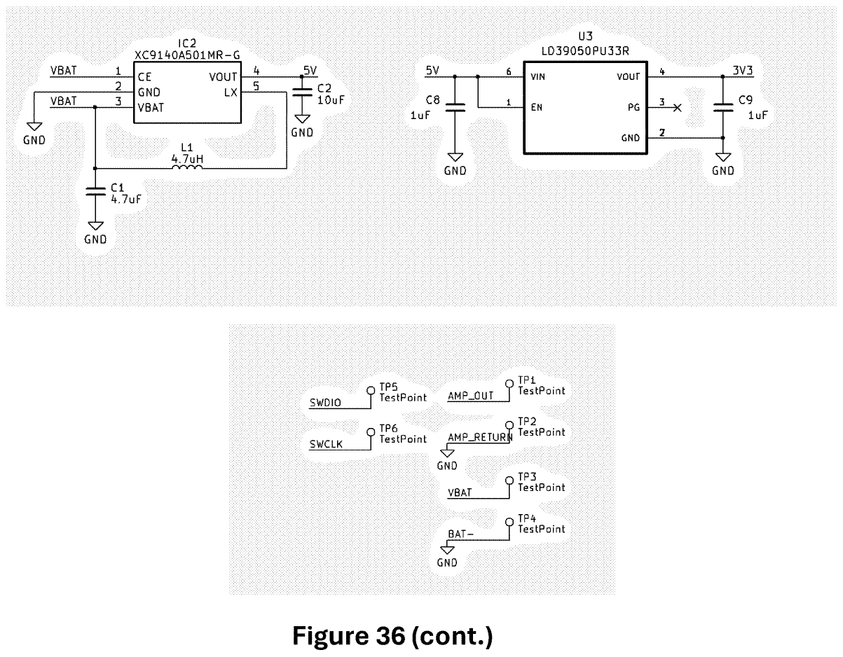

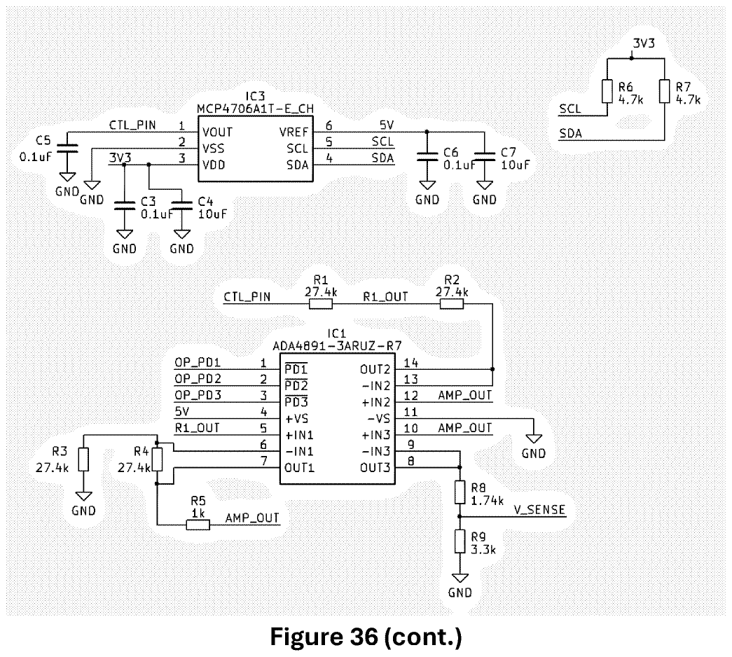

To enable clinically translatable solutions, we developed the H-Pad system (FIG. 6), a sustainable H₂ delivery platform that integrates the MEA with a flexible and wireless PCB. The PCB provides stable current delivery, voltage measurement, and Bluetooth®-based communication (FIGS. 6 (a), 34, 35, and 38). The functional block diagrams of the PCB (FIG. 6a, FIG. 36) include a microchip that controls a digital-to-analog converter (DAC) and regulates current through a Howland circuit, ensuring controlled H₂ production.

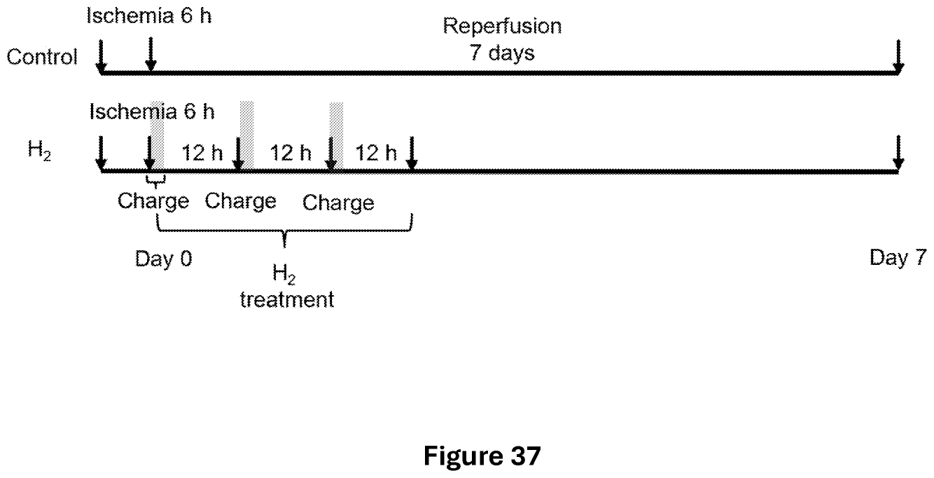

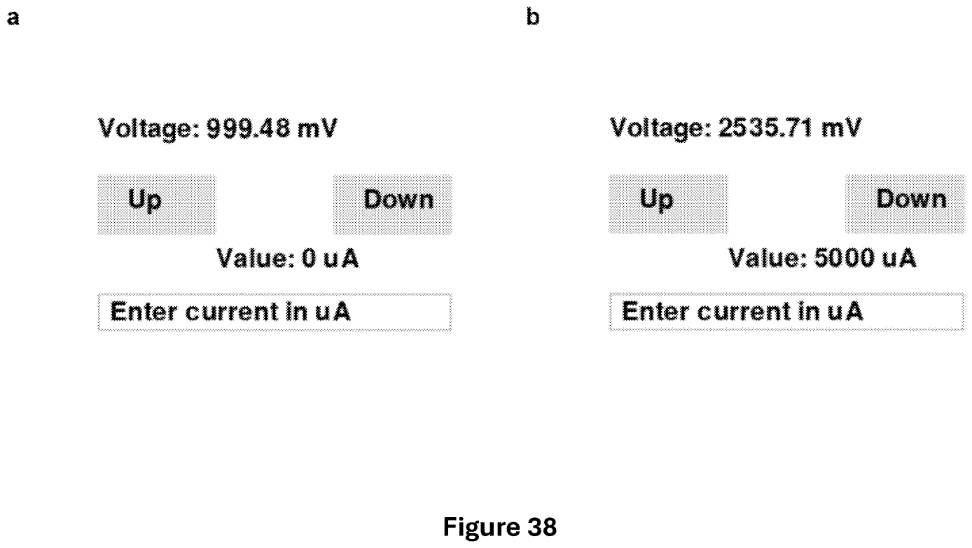

We adopted a previously reported protocol to induce pressure ulcers on mouse dorsal skin³⁶, using two magnets to compress the skin for 6 hours, followed by release to induce IR injury (FIG. 6 (c), FIG. 37). The experimental group (H₂ group, five mice) received H-Pad treatment (FIG. 6 (d)), while the control group (five mice) received only Tegaderm™ film coverage. A user interface (FIG. 38) was developed to control the system, delivering 5 mA current for 6 minutes, repeated thrice every 12 hours during the first 36 hours post-injury (FIG. 37). This treatment schedule specifically targeted the critical ROS surge post reperfusion. Visual confirmation of active H₂ generation was observed via bubble formation beneath the polyimide film (FIG. 6 (c)).

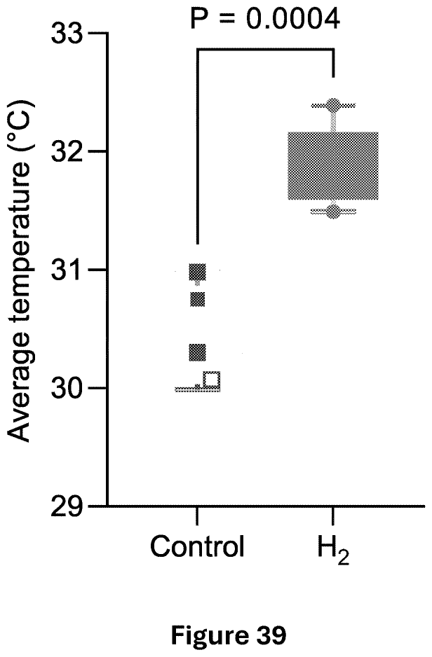

Wound assessment over 7 days revealed that H₂-treated sites showed transient redness on days 2 and 4, followed by reduced wound area and recovery (FIGS. 6 (e) and (h)). In contrast, control sites exhibited progressive blanching, ringed inflammation, and necrosis. Thermal imaging demonstrated higher local temperatures in H₂-treated wounds (FIGS. 6 (f) and (g) and FIG. 39), indicating improved perfusion and metabolic function.

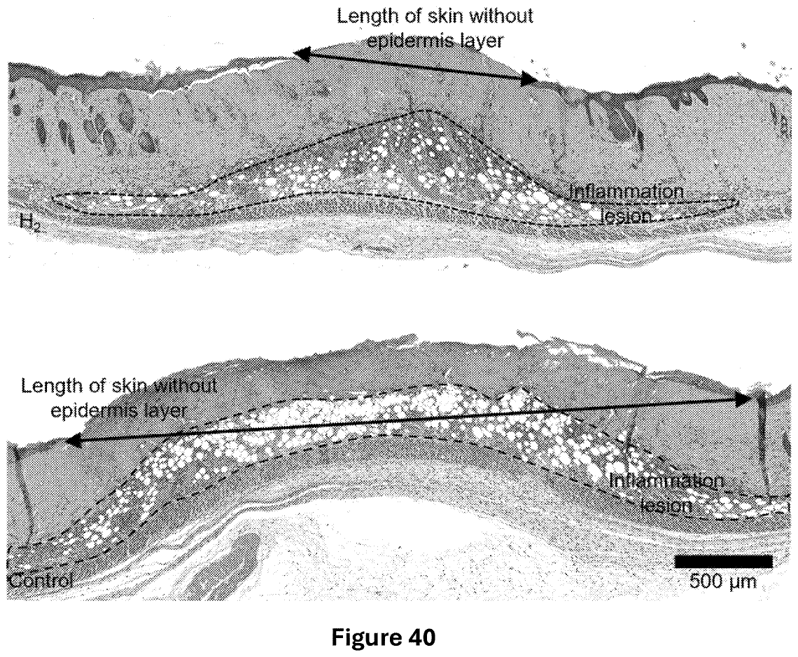

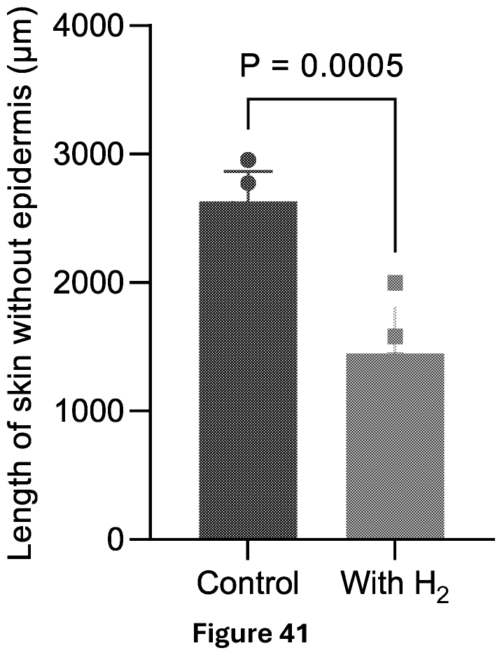

Hematoxylin and Eosin (H&E) staining revealed pronounced differences in tissue architecture and inflammatory responses between the H₂-treated and control groups (FIG. 6 (i)). The control group displayed severe dermal and epidermal damage with extensive edema and fluid accumulation, indicative of an exacerbated inflammatory response. In contrast, the H₂-treated group exhibited markedly reduced tissue disruption, diminished edema, smaller inflammatory lesions (FIG. 6 (k), FIG. 40), increased dermal thickness (FIG. 6 (j)), and evidence of epidermal regeneration (FIG. 41).

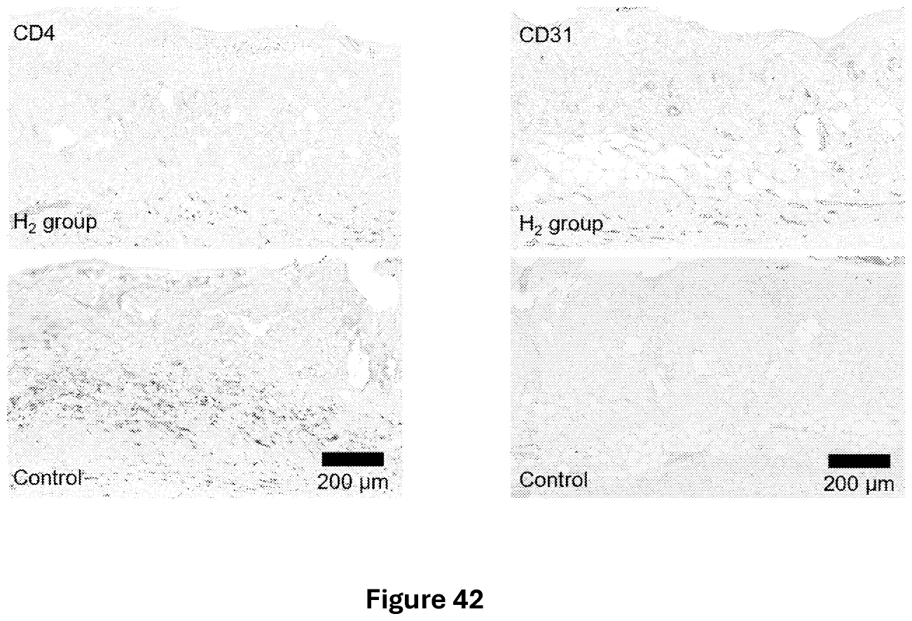

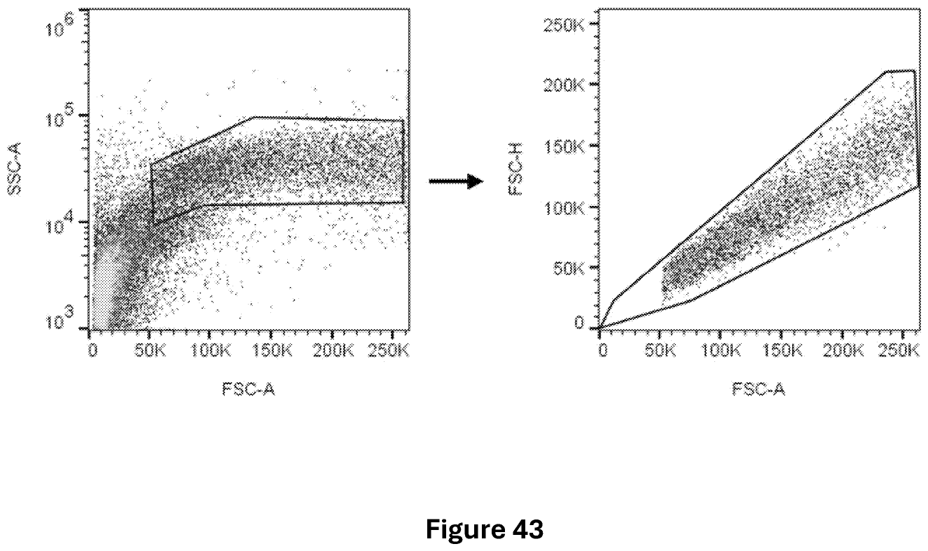

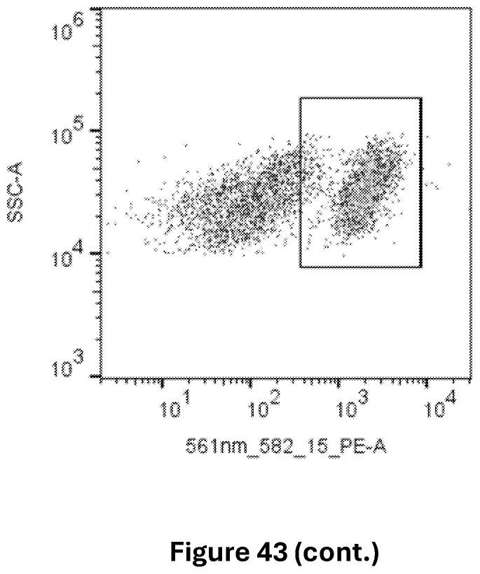

Immunohistochemical (IHC) staining for CD4 revealed fewer CD4+ T-cells in the H₂ group, indicating reduced inflammation (FIGS. 6 (l) and (m) and FIG. 42), while IHC for CD31 showed significantly increased neovascularization in H₂-treated tissue (FIGS. 6 (n) and (o) and FIG. 42). Collectively, these results demonstrate that H₂-based therapy attenuates ROS-induced damage, moderates inflammation, and promotes vascularization, offering a promising intervention for pressure ulcer management.

### CONCLUSION

This study presents a hydrogel-based electrochemical system that integrates controlled HER with localized gas storage and sustained release, providing a great platform in gas storage and transport for biomedical applications. We thoroughly reveal the influence of hydrogel polymer composition and mechanical properties on electrochemical kinetics, gas morphologies and gas storage. In vitro, this electrochemical configuration significantly reduces ROS damage, enhancing the viability of both CMs and keratinocytes under oxidative stress. Ex vivo, the system mitigates myocardial infarction in IR injured hearts, restoring electrical and contractile functions. In vivo, a wireless and portable H-Pad system minimizes skin damage in a pressure ulcer model, improving metabolic activity, reducing inflammation, and promoting neovascularization.

These findings underscore the potential of a hydrogel electrochemical system for continuously delivering therapeutic chemicals. Beyond H₂ therapy, this strategy could be adapted for other bioactive gases, such as oxygen therapy^{37,38} or combined with drug-loaded hydrogels, offering precise, tunable release kinetics. Recent advances in wearable and wireless bioelectronics and biosensing platform could be integrated to detect the skin metabolism and therapeutic conditions^{39,40}. Future efforts will focus on refining the device's electronics, enhancing wearability, integrating autonomous sensors for real-time feedback, and ensuring long-term biocompatibility.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

### REFERENCES

1. Cullen, D. A. et al. New roads and challenges for fuel cells in heavy-duty transportation. Nat. Energy 6, 462-474 (2021).
2. Li, M. et al. Environment Molecules Boost the Chemoselective Hydrogenation of Nitroarenes on Cobalt Single-Atom Catalysts. ACS Catal. 12, 11960-11973 (2022).
3. Ohsawa, I. et al. Hydrogen acts as a therapeutic antioxidant by selectively reducing cytotoxic oxygen radicals. Nat. Med. 13, 688-694 (2007).
4. Gunathilake, C. et al. A comprehensive review on hydrogen production, storage, and applications. Chem. Soc. Rev. 53, 10900-10969 (2024).
5. Nie, C. et al. Hydrogen gas inhalation alleviates myocardial ischemia-reperfusion injury by the inhibition of oxidative stress and NLRP3-mediated pyroptosis in rats. Life Sci. 272, 119248 (2021).
6. Zhang, Y. et al. Effects of hydrogen-rich water on depressive-like behavior in mice. Sci. Rep. 6, 23742 (2016).
7. Ge, L., Yang, M., Yang, N.-N., Yin, X.-X. & Song, W.-G. Molecular hydrogen: a preventive and therapeutic medical gas for various diseases. Oncotarget 8, 102653-102673 (2017).
8. Chen, S. et al. Photocatalytic glucose depletion and hydrogen generation for diabetic wound healing. Nat. Commun. 13, 5684 (2022).
9. Chen, H. et al. Symbiotic Algae-Bacteria Dressing for Producing Hydrogen to Accelerate Diabetic Wound Healing. Nano Lett. 22, 229-237 (2022).
10. Yang, N. et al. Magnesium galvanic cells produce hydrogen and modulate the tumor microenvironment to inhibit cancer growth. Nat. Commun. 13, 2336 (2022).
11. Zhou, T. et al. 3D printable high-performance conducting polymer hydrogel for all-hydrogel bioelectronic interfaces. Nat. Mater. 22, 895-902 (2023).
12. Shen, Q. et al. Liquid metal-based soft, hermetic, and wireless-communicable seals for stretchable systems. Science 379, 488-493 (2023).
13. Liu, L., Chakma, A. & Feng, X. Gas permeation through water-swollen hydrogel membranes. J. Membr. Sci. 310, 66-75 (2008).
14. Smiljanić, M. et al. Improving the HER Activity and Stability of Pt Nanoparticles by Titanium Oxynitride Support. ACS Catal. 12, 13021-13033 (2022).
15. Cowley, B. A. & Woodward*, and B. A Healthy Future: Platinum in Medical Applications: Platinum group metals enhance the quality of life of the global population. Platin. Met. Rev. 55, 98-107 (2011).
16. Wang, Y. et al. Monolayered Platinum Nanoparticles as Efficient Electrocatalysts for the Mass Production of Electrolyzed Hydrogen Water. Sci. Rep. 10, 10126 (2020).
17. Lu, Z. et al. Ultrahigh Hydrogen Evolution Performance of Under-Water “Superaerophobic” MoS2 Nanostructured Electrodes. Adv. Mater. 26, 2683-2687 (2014).
18. Zhao, X., Ren, H. & Luo, L. Gas Bubbles in Electrochemical Gas Evolution Reactions. Langmuir 35, 5392-5408 (2019).
19. Mazloomi, S. K. & Sulaiman, N. Influencing factors of water electrolysis electrical efficiency. Renew. Sustain. Energy Rev. 16, 4257-4263 (2012).
20. Watanabe, S. et al. Protective effect of inhalation of hydrogen gas on radiation-induced dermatitis and skin injury in rats. J. Radiat. Res. (Tokyo) 55, 1107-1113 (2014).
21. Gao, Q. et al. Molecular hydrogen increases resilience to stress in mice. Sci. Rep. 7, 9625 (2017).
22. de Almeida, A. J. P. O. et al. ROS: Basic Concepts, Sources, Cellular Signaling, and its Implications in Aging Pathways. Oxid. Med. Cell. Longev. 2022, U.S. Pat. No. 1,225,578 (2022).
23. Minutoli, L. et al. ROS-Mediated NLRP3 Inflammasome Activation in Brain, Heart, Kidney, and Testis Ischemia/Reperfusion Injury. Oxid. Med. Cell. Longev. 2016, U.S. Pat. No. 2,183,026 (2016).
24. Heusch, G. Myocardial ischaemia-reperfusion injury and cardioprotection in perspective. Nat. Rev. Cardiol. 17, 773-789 (2020).
25. Hausenloy, D. J. & Yellon, D. M. Ischaemic conditioning and reperfusion injury. Nat. Rev. Cardiol. 13, 193-209 (2016).
26. Herr, D. J., Aune, S. E. & Menick, D. R. Induction and Assessment of Ischemia-reperfusion Injury in Langendorff-perfused Rat Hearts. J. Vis. Exp. JoVE 52908 (2015).
27. Heusch, G. Myocardial ischaemia-reperfusion injury and cardioprotection in perspective. Nat. Rev. Cardiol. 17, 773-789 (2020).
28. Bugger, H. & Pfeil, K. Mitochondrial ROS in myocardial ischemia reperfusion and remodeling. Biochim. Biophys. Acta BBA—Mol. Basis Dis. 1866, 165768 (2020).
29. Vogel, B., Mehta, S. R. & Mehran, R. Reperfusion strategies in acute myocardial infarction and multivessel disease. Nat. Rev. Cardiol. 14, 665-678 (2017).
30. Padula, W. V. & Delarmente, B. A. The national cost of hospital—acquired pressure injuries in the United States. Int. Wound J. 16, 634-640 (2019).
31. Bauer, K., Rock, K., Nazzal, M., Jones, O. & Qu, W. Pressure Ulcers in the United States' Inpatient Population From 2008 to 2012: Results of a Retrospective Nationwide Study. Ostomy. Wound Manage. 62, 30-38 (2016).
32. Nussbaum, S. R. et al. An Economic Evaluation of the Impact, Cost, and Medicare Policy Implications of Chronic Nonhealing Wounds. Value Health J. Int. Soc. Pharmacoeconomics Outcomes Res. 21, 27-32 (2018).
33. Boyko, T. V., Longaker, M. T. & Yang, G. P. Review of the Current Management of Pressure Ulcers. Adv. Wound Care 7, 57-67 (2018).
34. Gawlitta, D., Oomens, C. W. J., Bader, D. L., Baaijens, F. P. T. & Bouten, C. V. C. Temporal differences in the influence of ischemic factors and deformation on the metabolism of engineered skeletal muscle. J. Appl. Physiol. 103, 464-473 (2007).
35. Kumar, S., Theis, T., Tschang, M., Nagaraj, V. & Berthiaume, F. Reactive Oxygen Species and Pressure Ulcer Formation after Traumatic Injury to Spinal Cord and Brain. Antioxidants 10, 1013 (2021).
36. Stadler, I., Zhang, R.-Y., Oskoui, P., Whittaker, M. B. S. & Lanzafame, R. J. Development of a Simple, Noninvasive, Clinically Relevant Model of Pressure Ulcers in the Mouse. J. Invest. Surg. 17, 221-227 (2004).
37. Lee, I. et al. Electrocatalytic on-site oxygenation for transplanted cell-based-therapies. Nat. Commun. 14, 7019 (2023).
38. Krishnan, S. R. et al. A wireless, battery-free device enables oxygen generation and immune protection of therapeutic xenotransplants in vivo. Proc. Natl. Acad. Sci. 120, e2311707120 (2023).
39. Wang, M. et al. A wearable electrochemical biosensor for the monitoring of metabolites and nutrients. Nat. Biomed. Eng. 6, 1225-1235 (2022).
40. Jiang, Y. et al. Wireless, closed-loop, smart bandage with integrated sensors and stimulators for advanced wound care and accelerated healing. Nat. Biotechnol. 41, 652-662 (2023).
41. Park, Y. J. et al. Electrodeposition of High-Surface-Area IrO2 Films on Ti Felt as an Efficient Catalyst for the Oxygen Evolution Reaction. Front. Chem. 8, 593272 (2020).
42. Li, P. et al. Monolithic silicon for high spatiotemporal translational photostimulation. Nature 626, 990-998 (2024).
43. Prominski, A. et al. Porosity-based heterojunctions enable leadless optoelectronic modulation of tissues. Nat. Mater. 21, 647-655 (2022).
44. Shi, J. et al. Monolithic-to-focal evolving biointerfaces in tissue regeneration and bioelectronics. Nat. Chem. Eng. 1, 73-86 (2024).

What is claimed:

1. An electrochemical cell, comprising:
a hydrogel;
a membrane electrode assembly (MEA) embedded in the hydrogel, the MEA having a first mesh electrode and a second mesh electrode; and
a proton exchange membrane (PEM) disposed between the first mesh electrode and the second mesh electrode.

2. The electrochemical cell of claim 1, wherein the hydrogel is a polyvinyl alcohol (PVA) hydrogel.

3. The electrochemical cell of claim 2, wherein the PVA hydrogel comprises a polymer concentration between about 3% and about 10%.

4. The electrochemical cell of claim 1, wherein the first mesh electrode comprises a titanium-platinum alloy (Ti—Pt) and the second mesh electrode comprises a Ti—IrO₂ electrode.

5. The electrochemical cell of claim 1, wherein the PEM comprises a sulfonated tetrafluoroethylene-based fluoropolymer-copolymer.

6. A system for generating and delivering molecular hydrogen, comprising:
a) an electrochemical cell comprising:
a hydrogel,
a membrane electrode assembly (MEA) embedded in the hydrogel, the MEA having a first mesh electrode and a second mesh electrode, and
a proton exchange membrane (PEM) disposed between the first mesh electrode and the second mesh electrode;
b) an adhesive coupled with a first side of the electrochemical cell, wherein the adhesive secures the electrochemical cell to a tissue of a subject; and
c) a wireless electronic circuit coupled to the electrochemical cell on a second side opposite the first side of the electrochemical cell.

7. The system of claim 6, wherein the adhesive comprises an adherent film.

8. The system of claim 7, wherein the adherent film comprises an acrylic-adhesive polyurethane film.

9. The system of claim 6, wherein the first mesh electrode comprises a titanium-platinum alloy (Ti—Pt) and the second mesh electrode comprises an iridium-titanium oxide (Ti—IrO₂) electrode.

10. The system of claim 6, wherein the wireless electronic circuit is a flexible printed circuit board (PCB).

11. The system of claim 10, wherein the PCB includes a microchip that is operably coupled to the PCB having a microcontroller, and wherein the microcontroller comprises one or more processors programmed to perform microcontroller operations including:
controlling a digital-to-analog converter (DAC) to regulate current through a Howland circuit contained on the PCB to the MEA of the electrochemical cell.

12. The system of claim 11, wherein controlling the DAC to regulate current through the Howland circuit contained on the PCB comprises ensuring controlled H₂ production.

13. The system of claim 11, wherein the one or more processors are programmed to perform microcontroller operations further including:
recording a voltage measurement from the MEA of the electrochemical cell.

14. The system of claim 11, wherein the one or more processors are programmed to perform microcontroller operations further including:
operating a communication system of the PCB.

15. The system of claim 10, wherein the PCB further includes a battery and a voltage regulator.

16. The system of claim 6, further comprising:
a polyimide film disposed between the electrochemical cell and the wireless electronic circuit.

17. The system of claim 6, further comprising:
a housing, wherein the housing encompasses the wireless electronic circuit.

18. A method for generating, storing, and delivering molecular hydrogen (H₂) to a tissue of a subject, the method comprising:
a) facilitating a hydrogen evolution reaction (HER) within an electrochemical cell to generate H₂, the electrochemical cell comprising:
a hydrogel,
a membrane electrode assembly (MEA) embedded in the hydrogel, the MEA having a first mesh electrode and a second mesh electrode, and
a proton exchange membrane (PEM) disposed between the first mesh electrode and the second mesh electrode;
b) storing, within the hydrogel of the electrochemical cell, the generated H₂;
c) regulating delivery of the stored H₂ from the hydrogel to a tissue of the subject using a wireless electronic circuit attached to the electrochemical cell.

19. The method of claim 18, wherein the wireless electronic circuit includes an operably coupled microchip having a microcontroller, and wherein the microcontroller comprises one or more processors programmed to perform microcontroller operations including:
controlling a digital-to-analog converter (DAC) to regulate current through a Howland circuit contained on the wireless electronic circuit to the MEA of the electrochemical cell.

20. The method of claim 19, wherein controlling the DAC to regulate current through the Howland circuit contained on the wireless electronic circuit comprises ensuring the facilitation of the HER for controlling H₂ generation.
